(19) United States
(12) Patent Application Publication
(10) Pub. No.: US 2017/0224679 A1
(43) Pub. Date: Aug. 10, 2017

(54) Use of CXCR2 Antagonists For The Prevention and/or Treatment of Chemotherapy Induced Peripheral Neuropathy (CIPN)

(71) Applicant: GlaxoSmithKline Intellectual Property Development Limited, Middlesex (GB)

(72) Inventors: Ebere F. IGBOKO, Brentford (GB); Paul Bryan WREN, Uxbridge (GB)

(21) Appl. No.: 15/500,758

(22) PCT Filed: Jul. 27, 2015
(86) PCT No.: PCT/EP2015/067132

§ 371 Date: Jan. 31, 2017

(30) Foreign Application Priority Data
Jul. 31, 2014 (CN) PCT/CN2014/083380

(51) Int. Cl. A61K 31/495; A61K 45/06; A61K 31/282; A61K 31/341; A61K 31/277; A61K 31/40; A61K 31/17; A61K 31/445; A61K 31/351

(52) U.S. Cl. CPC A61K 31/495; A61K 31/445; A61K 45/06; A61K 31/282; A61K 31/351; A61K 31/277; A61K 31/40; A61K 31/17; A61K 31/341

(57) ABSTRACT

The invention relates to the use of a CXCR2 antagonist for the prevention and/or treatment of chemotherapy induced peripheral neuropathy (CIPN).

### FIELD OF INVENTION

The invention relates to the use of CXCR2 antagonists for the prevention and/or treatment of chemotherapy induced peripheral neuropathy (CIPN).

### BACKGROUND OF THE INVENTION

CIPN is defined as the damage to the peripheral nervous system experienced by patients receiving chemotherapy treatment regimens. CIPN is a prevalent major dose-limiting side effect of many chemotherapeutic agents, including platinum compounds (for example, oxaliplatin), taxanes, vinca alkaloids, thalidomide and newer agents such as bortezomib [Balayssac, Expert Opin. Drug Saf., 10, 407-417, 2011)]. This represents a significant limitation to treatment in many diverse cancers, as end-organ neurotoxicity and neuropathy can have a high impact on a patient's quality of life and require discontinuation of effective therapy.

Oxaliplatin is known to cause severe acute and chronic peripheral neuropathies [Cassidy and Misset, Semin. Oncol., 29, 11-20 (2002); Extra, Semin. Oncol., 25, 13-22 (1998); Pasetto, Crit. Rev. Oncol. Hematol., 59, 159-168 (2006); and Quasthoff and Hartung, J. Neurol., 249, 9-17 (2002)]. Oxaliplatin causes degeneration of myelinated fibers in the rat sciatic nerve in the late phase after repeated treatment [Kawashiri, Eur. J. Pain, 15, 344 (2011)] and chronic treatment with oxaliplatin induces shrinkage of dorsal root ganglion (DRG) neurons and axonal damage of myelinated fibers in mouse models [Renn, Mol. Pain, 7, 29 (2011); Cavaletti, Eur. J. Cancer, 37, 2457 (2001)]. Early alterations of sensory endpoints in rodents has been seen in oxaliplatin models, resulting in increased sensitivity to thermal (Neuropharmacology, 79 (2014) 432-443; Pain, 101 (2003) 65-77) and mechanical stimuli (Neuropharmacology, 79 (2014) 432-443; Brain Research, 784(1998)154-162) using cold acetone stimuli and von Frey filament, respectively.

For example, oxaliplatin-containing treatment regimens (e.g. 85 mg/m² every 2 weeks) produce an immediate ‘cold’ sensitive transient paraesthesia and limb muscular spasm in 95% of patients that develops into a symmetric, axonal, sensory distal primary neuropathy without motor involvement [Argyriou, Cancer Treatment Reviews, 43, 368-377 (2008)].

Oxaliplatin uptake and platinum accumulation within the DRG and its sensory neurons is a major determinant of the neurotoxicity of oxaliplatin (Jong, J. Pharmacol. Exp. Ther., 338(2):537-47 (2011). In addition, inflammatory cascade activation plays a role in the initiation and progression of CIPN with immune cell infiltration into the injured neuronal environment [Wang, Cytokine, 59, 3-9 (2012)].

The pro-inflammatory chemokine receptor CXCR2 is expressed in sensory neurons and its ligands have been implicated in regulating increases in sodium and potassium currents that govern neuronal excitability [Wang, Mol. Pain, 24, 38 (2008); Yang, Mol. Pain, 5, 26 (2009)]. In peripheral neuronal injuries, the recruitment of CXCR2+pro-inflammatory secreting immune cells in rodents is also known to be involved in some acute and persistent pain states which are blocked by CXCR2 antagonism [Manjavachi, Eur. J. Pain, 14, 23-31 (2010); Kiguchi, J. Pharmacol. Exp. Ther., 340, 577-587 (2012); Stadtmann & Zarbock, Front. Immunol., 3, 263 (2012)]. CXCR2 ligands have been shown to regulate the function of TRPv1 channels [Dong, Neurosci. Bull., 28, 155-164 (2012)] involved in nociceptive processing and stimulate calcium influx and release of the pain mediating peptide calcitonin gene-related peptide (CGRP) in sensory neurons (Qin, J. Neurosci. Res., 82, 51-62 (2005). Human peripheral nerve explants and Schwann cell cultures express [Ozaki, NeuroReport, 19, 31-35 (2008)] and secrete CXCR2 pro-inflammatory cytokines like IL-8 [Rutkowski, J. Neuroimmunol., 101, 47-60 (1999)] which is significantly elevated in diabetic and alcoholic neuropathies and in length dependent small fiber neuropathy [AboEIAsar, Cytokine, 59, 86-93 (2012)]; (Michalowska-Wender, Folia Neuropathol., 45, 78-81 (2007); Üçeyler, Neurology, 74, 1806 (2010)]. The neuronal CXCR2 receptor system has also been shown to regulate re-myelination [Veenstra & Ransohoff, J. Neuroimmunol., 246, 1-9 (2012)] and synaptic plasticity (Xiong, J. Neurosci. Res., 71, 600-607 (2003) processes that govern neuronal communication.

The CXCR2 receptor and its ligands are also upregulated in colorectal cancer and have been implicated in chemoresistance [Acharyya, Cell, 150, 165-178 (2012)], tumor growth, vessel formation, cancer cell proliferation and neutrophil recruitment to the tumor microenvironment [Verbeke, Cytokine & Growth Factor Review, 22, 345-358 (2012)].

There are currently no approved treatment options for the prevention and/or treatment of CIPN.

### BRIEF SUMMARY OF THE INVENTION

According to a first aspect, the invention provides a CXCR2 antagonist for use in the prevention and/or treatment of CIPN.

According to a second aspect, the invention provides a method for the prevention and/or treatment of CIPN in a human in need thereof comprising administering a CXCR2 antagonist.

According to a third aspect, the invention provides the use of a CXCR2 antagonist in the manufacture of a medicament for the prevention and/or treatment of CIPN.

### BRIEF DESCRIPTION OF THE DRAWINGS

FIG. 1 is a graph showing the effect on paw withdrawal threshold (PWT) following chronic constriction injury (CCI) in rats.

FIG. 2 is a graph showing the effect on PWT following chronic constriction injury in mice.

FIG. 3 is a series of graphs showing the effect on mRNA levels in the sciatic nerve following chronic constriction injury in rats.

FIG. 4 is a series of graphs showing the effect on mRNA levels in the sciatic nerve following chronic constriction injury in mice.

FIG. 5 is a graph showing the effect on PWT following a single oxaliplatin injection in rats.

FIG. 6 is a graph showing the effect on PWT following a single oxaliplatin injection in mice.

FIG. 7 is a series of graphs showing the effect on mRNA levels in the left sciatic nerve following a single oxaliplatin injection in rats.

FIG. 8 is a series of graphs showing the effect on mRNA levels in the left sciatic nerve following a single oxaliplatin injection in mice.

FIG. 9 is a graph showing the effect on PWT following prophylactic treatment with Compound of Formula (A) in a rat model of chemotherapy induced neuropathy.

FIG. 10 is a graph showing the effect on oxaliplatin-reduced nerve conduction velocity in sciatic nerves in rats following treatment with Compound of Formula (A) or its vehicle.

FIG. 11 is a graph showing the effect on the weight of rats following treatment with Compound of Formula (A) or its vehicle.

FIG. 12 is a series of three photomicrographs showing the effect on the thickness of the myelin sheath of the sciatic nerve following treatment with Compound of Formula (A) in oxaliplatin-treated rats.

FIG. 13 is a bar chart showing the effect on the thickness of the myelin sheath of the sciatic nerve following treatment with Compound of Formula (A) in oxaliplatin-treated rats.

### DETAILED DESCRIPTION OF THE INVENTION

To date there has been no disclosure that CXCR2 modulation provides an effective prevention and/or treatment option for peripheral neuropathy, in particular CIPN. The proof of mechanism clinical study protocol described herein, is expected to provide human evidence that a potent and selective antagonist of CXCR2 can be an effective treatment option for CIPN. This proof of mechanism clinical study is to be conducted in patients suffering from colorectal cancer who are about to undertake cycles of an oxaliplatin containing chemotherapy treatment regimen. The treatment with a CXCR2 antagonist will be administered intermittently in and around the chemotherapy cycles. This regimen ensures the highest CXCR2 inhibition concomitantly with the highest systemic exposure of oxaliplatin. The study will measure a variety of relevant endpoints, before, during and after each cycle of chemotherapy. The primary endpoint will be the measurement of peripheral nerve excitability using neurophysiology tracking techniques. The acute increase of sensory nerve excitability (SE) caused by oxaliplatin containing treatment regimens, proposed to be caused by a dysfunction of nodal axonal voltage-gated sodium channels [Krishnan, Muscle Nerve, 32, 51-60 (2005)] is detectable earlier than established neurophysiological nerve conduction velocity markers and has been suggested to predict the occurrence and severity of oxaliplatin induced peripheral neuropathy [Park, Brain, 132, 10, 2712-23 (2009)]. A 15% increase from baseline in SE before the 5th cycle of oxaliplatin enabled the detection of 80% of cases with moderate to severe neurotoxicity by the end of the 12th cycle of oxaliplatin treatment.

Furthermore, the proof of mechanism clinical study protocol has been designed to provide evidence that a potent and selective CXCR2 antagonist is effective for the prevention and/or treatment of CIPN by capturing patient outcome information in relation to changes in sensory nerve excitability over the course of oxaliplatin cycles.

In addition, pre-clinical studies of CXCR2 antagonists have demonstrated no detrimental effect of CXCR2 inhibition on the antiproliferative action of oxaliplatin [Ning, Mol. Cancer Ther., 11, 1353-1364 (2012)]. The proof of mechanism study will also monitor effectiveness of the oxaliplatin containing treatment regimen on cancer related endpoints.

Therefore according to a first aspect, the invention provides a CXCR2 antagonist for use in the prevention and/or treatment of CIPN.

As used herein, the term CXCR2 antagonist means a compound that inhibits agonist-mediated responses at the CXCR2 receptor.

As used herein the term prevention means stopping nerve damage and/or consequential nerve dysfunction (as measured by neurophysiological tracking techniques) completely or slowing down the progression of nerve damage and/or change in nerve function. It will be appreciated that prevention covers a) the situation where no damage, or substantially no damage, occurs to a healthy nerve by the chemotherapeutic agent; b) the situation where a damaged nerve (caused by for example by an earlier cycle of chemotherapy) is not damaged, or substantially not damaged, further by the chemotherapeutic agent; and c) the situation where the damage to a nerve (caused by the cumulative effect of earlier cycles of chemotherapy) is reduced compared to any previous cycle of chemotherapy.

As used herein the term treatment means reversal or partial reversal of CIPN wherein the nerve damage and/or consequential nerve dysfunction (as measured by neurophysiological tracking techniques) is repaired or reversed leading to an improvement in nerve function, thereby reducing the symptoms of neuropathy.

CIPN is unique amongst neuropathies. In most other neuropathies, such as diabetic neuropathy and alcoholic neuropathy, the neuropathic symptoms are experienced some time after the insult/nerve damage has occurred. The likelihood of the occurrence of neuropathy in patients undergoing chemotherapy, particularly the high incidence of neuropathy experienced [Decision Resources LLC, Kantar Health, Oncology Analyser, Intrinsiq Database; De Gramont, J. Clin. Oncol., 18, 2938-2947 (2000)] with oxaliplatin chemotherapy, means that administering a CXCR2 antagonist before or at the same time as the chemotherapeutic agent is administered has the potential to prevent acute occurrences of neuropathy occurring and to prevent the establishment of a chronic lasting neuropathic condition. The proof of mechanism clinical study will measure this preventative aspect through evidence of modulation of nerve excitability measures and their suggested predictive translation to patient outcomes. In an embodiment, the invention provides a CXCR2 antagonist for use in the prevention of CIPN.

As discussed above many chemotherapeutic agents are known to cause CIPN, for example platinum compounds (for example, oxaliplatin), taxanes, vinca alkaloids, thalidomide and bortezomib. In an embodiment the CIPN is caused by chemotherapy comprising a platinum-containing chemotherapeutic agent. In an embodiment the CIPN is caused by chemotherapy comprising oxaliplatin. In an embodiment the CIPN is caused by a platinum-containing chemotherapeutic agent. In a further embodiment the CIPN is caused by oxaliplatin.

The suitability of a CXCR2 antagonist for use with the invention can be determined by evaluation of its potency, selectivity, absorption, metabolism, pharmacokinetics, toxicity etc, in accordance, with standard pharmaceutical practice.

The potency of the CXCR2 antagonist may be determined using readily available assay methods, such as Assays a), b), c) and d), described in the Experimental Section below.

Assay a) measures the radiolabeled binding of the human endogenous agonist IL-8 directly to the recombinantly expressed human CXCR2 receptor. Assay b) measures the functional activity to the recombinantly expressed human CXCR2 receptor measured downstream of the receptor itself through a reporter gene construct. Assay c) measures the functional activity to the recombinantly expressed human CXCR2 receptor measured downstream of the receptor itself through a reporter gene construct. Assay d) measures the cellular functional activity to the native expressed human CXCR2 in whole blood as measured downstream of the receptor itself through a Flow cytometry cell sorting protocol. In each assay, the inhibition of agonist mediated endpoints determines the antagonist potency. For a given antagonist, the potency may differ depending on the assay used. However the skilled pharmacologist will be able to readily compare results from the differing assays in determining potency.

In an embodiment, the CXCR2 antagonist has a pIC50 value against the CXCR2 receptor of greater than or equal to 7.0 as measured in a CXCR2 receptor binding assay. In a further embodiment, the CXCR2 antagonist has a pIC50 value against the CXCR2 receptor of greater than or equal to 7.8 as measured in a receptor binding assay.

In an embodiment, the CXCR2 antagonist has a pIC50 value against the CXCR2 receptor of greater than or equal to 7.0 as measured in assay a). In a further embodiment, the CXCR2 antagonist has a pIC50 value against the CXCR2 receptor of greater than or equal to 7.8 as measured in assay a).

In an embodiment, the CXCR2 antagonist has a pA2 value against the CXCR2 receptor of greater than or equal to 8.0 as measured in assay b). In a further embodiment, the CXCR2 antagonist has a pA2 value against the CXCR2 receptor of greater than or equal to 8.2 as measured in assay b).

In an embodiment, the CXCR2 antagonist has a pIC50 value against the CXCR2 receptor of greater than or equal to 8.0 as measured in assays c). In a further embodiment, the CXCR2 antagonist has a pIC50 value against the CXCR2 receptor of greater than or equal to 8.8 as measured in assays c).

In an embodiment, the CXCR2 antagonist has a pIC50 value against the CXCR2 receptor of greater than or equal to 5.0 as measured in assay d). In a further embodiment, the CXCR2 antagonist has a pIC50 value against the CXCR2 receptor of greater than or equal to 5.4 as measured in assay d).

The selectivity of the CXCR2 antagonist at the CXCR2 receptor over the CXCR1 receptor may be determined using Assays a) and e). Selectivity ratios may readily be determined by the skilled person, by ratio of corresponding IC50 values for the particular receptors concerned. In an embodiment, the CXCR2 antagonist has a selectivity for CXCR2 over CXCR1 of greater than or equal to 29 fold. In a further embodiment, the CXCR2 antagonist has a selectivity for CXCR2 over CXCR1 of greater than or equal to 50 fold. In an embodiment, the CXCR2 antagonist has a selectivity for CXCR2 over CXCR1 of greater than or equal to 100 fold.

Oral bioavailablity refers to the proportion of an orally administered drug that reaches the systemic circulation. The factors that determine oral bioavailability of a drug are solubility, membrane permeability, metabolic stability and possible involvement of active transporters. Typically, a screening cascade firstly uses in vitro studies to identify potential liability and then progress to in vivo assessment to determine oral bioavailability.

Dissolution, the solubilisation of the drug by the aqueous contents of the gastro-intestinal tract (GIT), can be predicted from in vitro solubility experiments conducted at appropriate pH to mimic the GIT. In an embodiment, the CXCR2 antagonist has a minimum solubility of 10 μg/ml.

Solubility can be determined by standard procedures known in the art such as described in Adv. Drug Deliv. Rev., 23, 3-25 (1997).

Membrane permeability refers to the rate of a compound passing through the biological membrane. Lipophilicity is a key property in predicting this and is defined by in vitro Log D_{7.4} measurements using organic solvents and buffer. In an embodiment the CXCR2 antagonist has a Log D_{7.4} of −2 to +4, more preferably −1 to +3. Log D_{7.4} may be determined by standard procedures known in the art such as described in J. Pharm. Pharmacol., 42:144 (1990).

Metabolic stability addresses the ability of the GIT or the liver to metabolise compounds during the absorption and elimination processes. Assay systems such as microsomes, hepatocytes etc are predictive of metabolic liability. In an embodiment, the CXCR2 antagonist shows metabolic stability in the assay system that is commensurate with a low to moderate hepatic extraction. Examples of assay systems and data manipulation are described in Curr. Opin. Drug Disc. Devel., 4, 36-44 (2001); Drug Met. Disp., 28,1518-1523 (2000).

Because of the interplay of the above processes, further evaluation that a drug will potentially be orally bioavailable in humans can be obtained by in vivo experiments in preclinical species. Absolute bioavailability is determined in these studies by administering the compound by the oral and intravenous routes. Examples of the assessment of oral bioavailability in animals can be found in Drug Met. Disp., 29, 82-87 (2001); J. Med Chem., 40, 827-829 (1997); Drug Met. Disp., 27, 221-226 (1999).

Suitable CXCR2 antagonists for use with the invention are disclosed in European patent publication EP1 558 259 B1 and EP2 009 992 B1. The contents of these publications, in particular the general formulae of the therapeutically active compounds of the claims and exemplified compounds therein, are incorporated herein in their entirety by reference thereto.

In an embodiment, the CXCR2 antagonist for use with the invention is selected from the group consisting of:

1-(4-chloro-2-hydroxy-3-(piperazin-1-ylsulfonyl)phenyl)-3-(2-chloro-3-fluorophenyl)urea of

Formula (A)

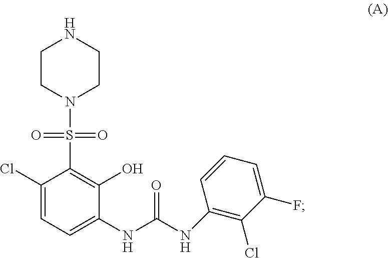 [SMILES: O=C(Nc1ccc(Cl)c(S(=O)(=O)N2CCNCC2)c1O)Nc1cccc(F)c1Cl]

1-(4-chloro-2-hydroxy-3-(piperidin-3-ylsulfonyl)phenyl)-3-(3-fluoro-2-methylphenyl)urea of Formula (B)

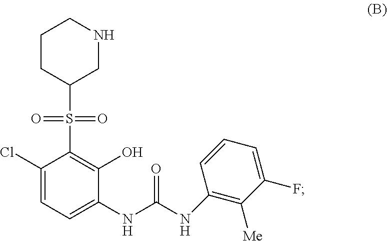 [SMILES: Cc1c(F)cccc1NC(=O)Nc1ccc(Cl)c(S(=O)(=O)C2CCCNC2)c1O]

1-(4-chloro-2-hydroxy-3-(((R)-3-methyltetrahydrofuran-3-yl)sulfonyl)phenyl)-3-((R)-2-methylcyclopent-2-en-1-yl)urea of Formula (C)

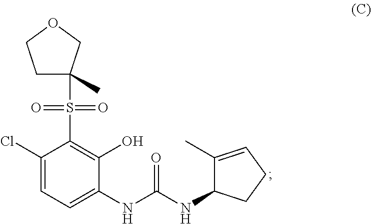 [SMILES: CC1=CCC[C@H]1NC(=O)Nc1ccc(Cl)c(S(=O)(=O)[C@]2(C)CCOC2)c1O]

1-(4-chloro-3-((1,4-dimethylpiperidin-4-yl)sulfonyl)-2-hydroxyphenyl)-3-(2-chloro-3-fluorophenyl)urea of Formula (D)

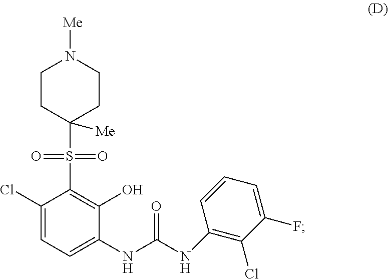 [SMILES: CN1CCC(C)(S(=O)(=O)c2c(Cl)ccc(NC(=O)Nc3cccc(F)c3Cl)c2O)CC1]

(R)-1-(4-chloro-2-hydroxy-3-((4-methyltetrahydro-2H-pyran-4-yl)sulfonyl)phenyl)-3-(2-chlorocyclopent-2-en-1-yl)urea of Formula (E)

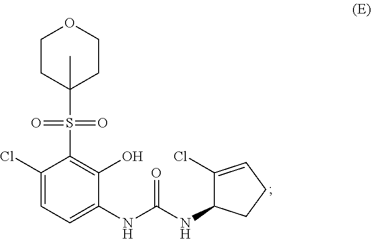

(R)-1-(3-(tert-butylsulfonyl)-4-cyano-2-hydroxyphenyl)-3-(2-methylcyclopent-2-en-1-yl)urea of Formula (F)

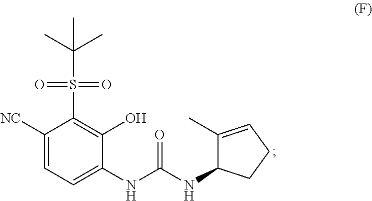 [SMILES: CC1=CCC[C@H]1NC(=O)Nc1ccc(C#N)c(S(=O)(=O)C(C)(C)C)c1O]

1-(4-chloro-2-hydroxy-3-((trans-3-(pyrrolidin-1-yl)cyclobutyl)sulfonyl)phenyl)-3-((R)-2-methylcyclopent-2-en-1-yl)urea of Formula (G)

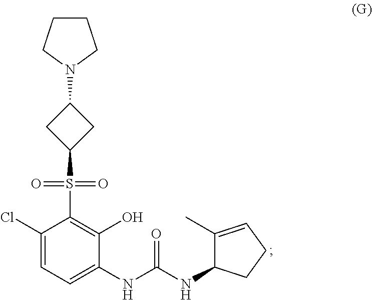 [SMILES: CC1=CCC[C@H]1NC(=O)Nc1ccc(Cl)c(S(=O)(=O)[C@H]2C[C@H](N3CCCC3)C2)c1O]

1-(4-chloro-3-((trans-3-(dimethylamino)cyclobutyl)sulfonyl)-2-hydroxyphenyl)-3-((R)-2-methylcyclopent-2-en-1-yl)urea of Formula (H)

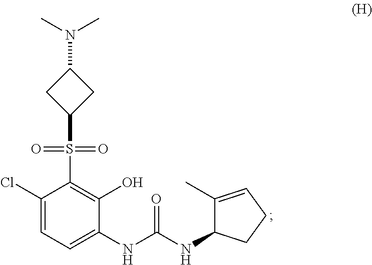

(R)-1-(4-chloro-3-((1,1-difluoroethyl)sulfonyl)-2-hydroxphenyl)-3-(2-methylcyclopent-2-en-1-yl)urea of Formula (I)

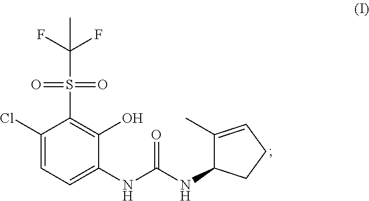 [SMILES: CC1=CCC[C@H]1NC(=O)Nc1ccc(Cl)c(S(=O)(=O)C(C)(F)F)c1O]

1-(4-chloro-2-hydroxy-3-(((S)-3-methyltetrahydrofuran-3-yl)sulfonyl)phenyl)-3-((R)-2-methylcyclopent-2-en-1-yl)urea of Formula (J)

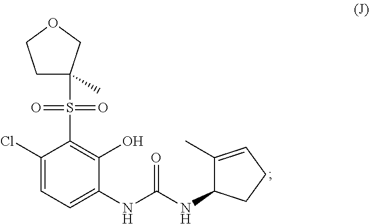 [SMILES: CC1=CCC[C@H]1NC(=O)Nc1ccc(Cl)c(S(=O)(=O)[C@@]2(C)CCOC2)c1O]

1-(4-chloro-2-hydroxy-3-(((S)-3-methyltetrahydrofuran-3-yl)sulfonyl)phenyl)-3-((R)-2-chlorocyclopent-2-en-1-yl)urea of Formula (K)

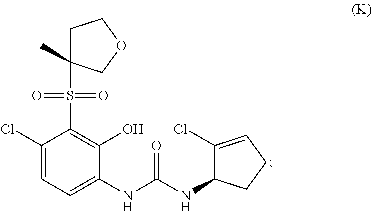 [SMILES: C[C@@]1(S(=O)(=O)c2c(Cl)ccc(NC(=O)N[C@@H]3CCC=C3Cl)c2O)CCOC1]

and

1-(4-chloro-2-hydroxy-3-(((R)-3-methyltetrahydrofuran-3-yl)sulfonyl)phenyl)-3-((R)-2-chlorocyclopent-2-en-1-yl)urea of Formula (L)

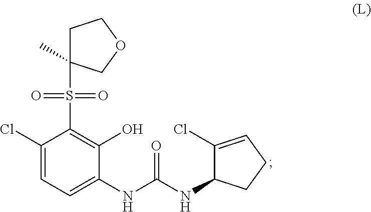 [SMILES: C[C@]1(S(=O)(=O)c2c(Cl)ccc(NC(=O)N[C@@H]3CCC=C3Cl)c2O)CCOC1]

or a pharmaceutically acceptable salt thereof.

Compound of Formula (A) may be prepared according to the method described in European Patent EP1 558 259 B1, Example 1.

Compound of Formula (B) may be prepared according to the method described in European Patent EP2 009 992 B1, Example 1.

Compounds of Formula (C), (D), (E), (F), (G), (H), (I), (J), (K) and (L) may be prepared according to the methods described below. See also for preparation International Patent Application PCT/EP2015/061618.

In a further embodiment, the CXCR2 antagonist is 1-(4-chloro-2-hydroxy-3-(piperazin-1-ylsulfonyl)phenyl)-3-(2-chloro-3-fluorophenyl)urea of Formula (A) or a pharmaceutically acceptable salt thereof

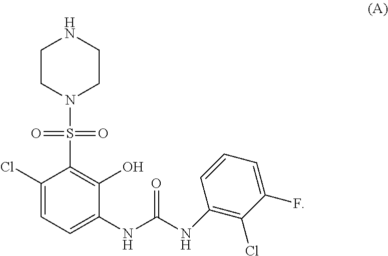 [SMILES: O=C(Nc1ccc(Cl)c(S(=O)(=O)N2CCNCC2)c1O)Nc1cccc(F)c1Cl]

In a further embodiment, the CXCR2 antagonist is 1-(4-chloro-2-hydroxy-3-(piperidin-3-ylsulfonyl)phenyl)-3-(3-fluoro-2-methylphenyl)urea of Formula (B) or a pharmaceutically acceptable salt thereof

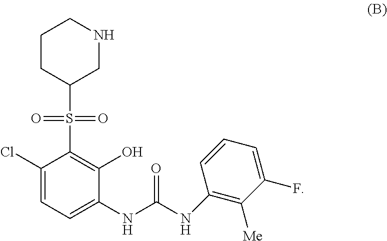 [SMILES: Cc1c(F)cccc1NC(=O)Nc1ccc(Cl)c(S(=O)(=O)C2CCCNC2)c1O]

In a further embodiment, the CXCR2 antagonist is 1-(4-chloro-2-hydroxy-3-(((R)-3-methyltetrahydrofuran-3-yl)sulfonyl)phenyl)-3-((R)-2-methylcyclopent-2-en-1-yl)urea of Formula (C) or a pharmaceutically acceptable salt thereof

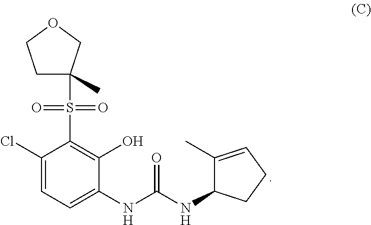 [SMILES: CC1=CCC[C@H]1NC(=O)Nc1ccc(Cl)c(S(=O)(=O)[C@]2(C)CCOC2)c1O]

In a further embodiment, the CXCR2 antagonist is 1-(4-chloro-2-hydroxy-3-(((S)-3-methyltetrahydrofuran-3-yl)sulfonyl)phenyl)-3-((R)-2-methylcyclopent-2-en-1-yl)urea of Formula (J) or a pharmaceutically acceptable salt thereof

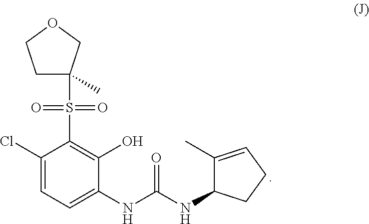 [SMILES: CC1=CCC[C@H]1NC(=O)Nc1ccc(Cl)c(S(=O)(=O)[C@@]2(C)CCOC2)c1O]

The CXCR2 antagonist can be administered alone but will generally be administered in a mixture with a suitable pharmaceutical excipient, diluent or carrier selected with regard to the intended route of administration and standard pharmaceutical practice.

The CXCR2 antagonist will normally, but not necessarily, be formulated into a pharmaceutical composition prior to administration to a patient by an appropriate route. Accordingly, in another aspect, the invention provides a pharmaceutical composition comprising a) a CXCR2 antagonist, and b) one or more pharmaceutically acceptable excipients for use in the prevention and/or treatment of CIPN.

A pharmaceutical composition of the invention may be prepared and packaged in bulk form wherein a compound of the invention is dispensed and then given to the patient (for example powders and syrups). Alternatively, the pharmaceutical compositions of the invention may be prepared and packaged as dosage forms wherein each physically discrete dosage form contains a compound of the invention. Accordingly, in another aspect, the invention provides dosage forms comprising pharmaceutical compositions of the invention. For the average human subject having a weight range of about 65 to 80 kg, each discrete dosage form typically contains from 5 mg to 100 mg of a compound of the invention. The skilled person will readily be able to determine the dosage levels required for a subject whose weight falls outside this range, such as children and the elderly.

The compositions of the invention will typically be formulated into dosage forms which are adapted for administration to the patient by the desired route of administration. For example, dosage forms include those adapted for (1) oral administration such as tablets, capsules, caplets, pills, lozenges, powders, syrups, elixirs, suspensions, solutions, emulsions, sachets and cachets; (2) parenteral administration such as sterile solutions, suspensions, implants and powders for reconstitution; (3) transdermal administration such as transdermal patches; (4) rectal and vaginal administration such as suppositories, pessaries and foams; (5) inhalation and intranasal such as dry powders, aerosols, suspensions and solutions (sprays and drops); (6) topical administration such as creams, ointments, lotions, solutions, pastes, drops, sprays, foams and gels; (7) ocular administration such as drops, ointment, sprays, suspensions and inserts; (8) buccal and sublingual administration such as lozenges, patches, sprays, drops, chewing gums and tablets. In an embodiment, the dosage form is adapted for oral administration. In an embodiment, the composition is adapted for oral administration.

As used herein, the term “pharmaceutically acceptable excipient” means a substance which does not appreciably react with the CXCR2 antagonist, nor result in an undesired effect on the therapeutic activity of the CXCR2 antagonist. Pharmaceutically acceptable excipients will vary depending upon the particular dosage form chosen. In addition, pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of the invention once administered to the patient from one organ, or portion of the body, to another organ, or portion of the body. Certain pharmaceutically acceptable excipients may be chosen for their ability to enhance patient compliance. Certain pharmaceutically acceptable excipients may be chosen for their ability to facilitate the release of the CXCR2 antagonist at the appropriate rate to treat the condition.

Pharmaceutically acceptable excipients for use with compositions adapted for oral administration include: diluents and fillers (such as lactose, sucrose, dextrose, mannitol, sorbitol, corn starch, potato starch, pre-gelatinized starch, cellulose, microcrystalline cellulose, calcium sulfate, and dibasic calcium phosphate); binders (such as starch, corn starch, potato starch, pre-gelatinized starch, gelatin, acacia, sodium alginate, alginic acid, tragacanth, guar gum, povidone, cellulose and hydroxypropyl methyl cellulose); disintegrants (such as starches, crospovidone, sodium starch glycolate, cros-carmellose, alginic acid and sodium carboxymethyl cellulose); lubricants (such as stearic acid, magnesium stearate, calcium stearate, and sodium dodecyl sulphate); glidants (such as talc and colloidal silicon dioxide); granulating agents; coating agents; wetting agents; solvents; co-solvents; suspending agents; emulsifiers; sweeteners; flavouring agents; flavour masking agents; colouring agents; anticaking agents; humectants; chelating agents; plasticizers; viscosity increasing agents; rate modifying agents; antioxidants; preservatives; stabilizers; surfactants; and buffering agents. The skilled artisan will appreciate that certain pharmaceutically acceptable excipients may serve more than one function and may serve alternative functions depending on how much of the excipient is present in the formulation and what other ingredients are present in the formulation. In an embodiment, the dosage forms adapted for oral administration such as tablets, capsules, caplets and pills, may be formulated for immediate-, delayed-, modified-, sustained-, pulsed- or controlled-release of the compound of the invention.

Skilled artisans possess the knowledge and skill in the art to enable them to determine pharmaceutically acceptable excipients in appropriate amounts for use with the CXCR2 antagonist. In addition, there are a number of resources that are available to the skilled artisan which describe pharmaceutically acceptable excipients and may be useful in selecting pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press). The pharmaceutical compositions of the invention may be prepared using techniques and methods known to those skilled in the art. Some of the methods commonly used in the art are described in Remington's Pharmaceutical Sciences (Mack Publishing Company).

The following formulation example is illustrative only and is not intended to limit the scope of the invention.

| Intra-granular | CXCR2 Antagonist 1-(4-chloro-2-hydroxy-3- | 68.60 | mg (50 mg |
|---|---|---|---|
| | (piperazin-1-ylsulfonyl)phenyl)-3-(2-chloro-3- | | free base) |
| | fluorophenyl)urea of Formula (A) | | |
| | Lactose monohydrate | 36.03 | mg |
| | MCC, Avicel PH101 | 30.00 | mg |
| | Hypromellose 2910 | 7.50 | mg |
| | Croscarmellose Sodium | 4.50 | mg |
| | Sterile water for irrigation, 100 percent | q.s. | |
| | Total | 146.63 | mg |
| Extra-granular | Croscarmellose Sodium | 2.25 | mg |
| | Magnesium Stearate RM147116 | 1.12 | mg |
| | Total | 150.00 | mg |
| Film Coat | Opadry film coating, white | 4.50 | mg |
| | Sterile water for irrigation, 100 percent | q.s. | |
| | Total | 154.50 | mg |

According to the invention, the CXCR2 antagonist prevents and/or treats CIPN caused by the administration of chemotherapeutic agents. Accordingly, the CXCR2 antagonist may be administered in combination with a chemotherapeutic agent. In an embodiment of the invention, the CXCR2 antagonist for the prevention and/or treatment of CIPN is combined with one or more primary chemotherapeutic agents, the active agent being selected from the following list: platinum compounds (for example, oxaliplatin), taxanes, vinca alkaloids, thalidomide and bortezomib. In a further embodiment, the primary chemotherapeutic agent is oxaliplatin.

Often chemotherapeutic agents are administered in combination with additional agents (eg, other chemotherapeutic agents, angiogenesis inhibitors, anti-emetics) that provide increased treatment options for effective patient compliant anticancer treatment inclusive of treating refractory populations. In a further embodiment of the invention, the CXCR2 antagonist is administered with a) a primary chemotherapeutic agent and b) an additional agent selected from the group consisting of bevacizumab, irinotecan, capecitabine, cetuximab, panitumumab, regorafenib and Ziv-aflibercept.

If a combination of active agents are administered, then they may be administered simultaneously, separately or sequentially. In an embodiment the CXCR2 antagonist is administered before or at the same time as the chemotherapeutic agent is administered.

It will be appreciated that the invention provides basis for the following further aspects. The embodiments specified hereinabove for the first aspect extend to these aspects:

a method for the prevention and/or treatment of CIPN in a human in need thereof comprising administering a CXCR2 antagonist;

the use of a CXCR2 antagonist in the manufacture of a medicament for the prevention and/or treatment of CIPN;

a pharmaceutical combination for the prevention and/or treatment of CIPN comprising a CXCR2 antagonist and an additional active agent as hereinabove defined;

a method for the prevention and/or treatment of CIPN in a human in need thereof comprising administering a pharmaceutical combination comprising a CXCR2 antagonist and an additional active agent as hereinabove defined;

the use of a pharmaceutical combination for the manufacture of a medicament for the prevention and/or treatment of CIPN in patients comprising a CXCR2 antagonist and an additional active agent as hereinabove defined;

a kit for the prevention and/or treatment of CIPN, the kit comprising: a) a first pharmaceutical composition comprising a CXCR2 antagonist; b) a second composition comprising an additional active agent as hereinabove defined; and c) a container for the first and second compositions; and

the use of a kit in the manufacture of a medicament for the prevention and/or treatment of CIPN, the kit comprising: a) a first pharmaceutical composition comprising a CXCR2 antagonist; b) a second composition comprising an additional active agent as hereinabove defined; and c) a container for the first and second compositions.

Experimental Section

a) Receptor Binding Assay for CXCR2 and CXCR1

[¹²⁵I] IL-8 (human recombinant, IM249) was obtained from Amersham Corp., Arlington Heights, Ill., with specific activity 2000 Ci/mmol. All other chemicals were of analytical grade. High levels of recombinant human CXCR1 and CXCR2 receptors were individually expressed in Chinese hamster ovary (CHO) cells as described in Holmes, et al., Science, 1991, 253, 1278, incorporated herein to the extent required to perform the present assay. The Chinese hamster ovary membranes were prepared according to Haour, et al., J. Biol. Chem., 249 pp 2195-2205 (1974), incorporated herein to the extent required to perform the present assay, except that the homogenization buffer was changed to 40 mM Tris-HCL pH 7.5 containing 1 mM MgSO₄, 0.5 mM EDTA (ethylene-diaminetetra-acetic acid), 1 mM PMSF (α-toluene-sulphonyl fluoride), 2.5 mg/L Leupeptin and 0.1 mg/ml Aprotinin. Membrane protein concentration was determined using Bio-Rad Reagent using bovine serum albumin as a standard. All binding assays were conducted using Scintillation Proximity Assay (SPA assay) using wheatgerm agglutinin beads in a 96-well micro plate (optiplate 96, Packard) format. The CHO-CXCR2 and CHO-CXCR1 membranes were pre-incubated with the beads in binding buffer; 20 mM Bis Tris propane, pH 8 containing 25 mM NaCl, 1 mM MgSO₄, 0.1 mM EDTA at 4° C. for 30 min prior to assay. The compound was diluted in 100% DMSO at 20 times the final concentration (final 1 nM to 1000 nM and 5% DMSO). The assay was performed in 0.1 ml reaction buffer containing binding buffer, membranes pre-treated with wheatgerm agglutinin beads, various concentrations of compound, 5% DMSO, 0.04% CHAP, 0.0025% BSA and 0.225 nM [¹²⁵I] IL-8. The 96-well plates were incubated on a shaking platform for 1 hour. At the end of the incubation the plates were spun for 5 min at 2000 RPM, and counted in a Top Count counter.

A compound demonstrating a pIC50 value of >5 is considered active in the assay.

The Compound of Formula (A) gave a pIC50 value of 7.8 against the CXCR2 receptor and a value of 5.5 against the CXCR1 receptor. As measured in this assay, Compound of Formula (A) is therefore 188 fold selective for CXCR2 over CXCR1.

The Compound of Formula (B) gave a pIC50 value of 7.9 against the CXCR2 receptor and a value of 6.0 against the CXCR1 receptor. As measured in this assay, Compound of Formula (B) is therefore 78 fold selective for CXCR2 over CXCR1.

b) Calcium Mobilization Assay

CHO-K1 cells, stably expressing CXCR2 and Gαl6, were grown to 80% confluency in DMEM/F12 (HAM's) 1:1, w/10% FCS (heat inactivated), w/2 mM L-glutamine, w/0.4 mg/ml G418 while maintained at 37° C. in a 5% CO₂ incubator. Twenty four hours prior to conducting the assay, the cells were harvested and plated (40,000 cells per well) in a 96 well, black wall, clear bottom plate (Packard View) and returned to the CO₂ incubator. On the day of the assay, compounds were serially diluted in 100% DMSO to 300× the desired assay concentration. Growth media was aspirated from the cells and replaced with 100 μL of load media (EMEM with Earl's salts w/LGlutamine, 0.1% BSA, (Bovunlinar Cohen Fraction V from Seriologicals Corp.), 4 uM Fluo-4-acetoxymethyl ester fluorescent indicator dye (Fluo-4 AM, from Molecular Probes) and 2.5 mM probenecid) and incubated for 1 hour at 37° C. in a CO₂ incubator. Load media was aspirated and replaced with 100 μL of EMEM with Earl's salts w/L-Glutamine, 0.1% gelatin and 2.5 mM probenecid and incubated for an additional 10 min. Serially diluted compound (3 μL) in DMSO at 300× was transferred to a 96 well plate containing 297 micro liters of KRH (120 mM NaCl, 4.6 mM KCl, 1.03 mM KH₂PO₄, 25 mM NaHCO₃, 1.0 mM CaCl₂, 1.1 mM MgCl₂, 11 mM glucose, 20 mM HEPES (pH 7.4)) w/2.5 mM probenecid and 0.1% gelatin (compound now at 3×). The media was aspirated from the cells and the cells were washed 3 times with KRH w/2.5 mM probenecid, w/0.1% gelatin. KRH (100 μL) w/2.5 mM probenecid with 0.1% gelatin was added to wells then 50 μL of 3× compound in KRH w/2.5 mM probenecid and 0.1% gelatin was added to wells and incubated at 37° C. in the CO2 incubator for 10 min. The plates were placed onto FLIPR (Fluorometric Imaging Plate Reader, Molecular Devices, Sunnyvale Calif.) for analysis as described previously [Sarau et al., Mol. Pharmacol., September, 56(3), 657-63 (1999)]. The percent of maximal human IL-8 induced Ca²⁺ mobilization induced by 1.0 nM IL-8, an EC₈₀ conc. for CXCR2, was determined for each concentration of compound and the IC₅₀ calculated as the concentration of test compound that inhibits 50% of the maximal response induced by 1.0 nM IL-8.

As measured in this assay, the Compound of Formula (A) gave a pA2 value of 8.4 against the CXCR2 receptor.

As measured in this assay, the Compound of Formula (B) gave a pA2 value of 8.2 against CXCR2.

c) CXCR2 Tango Assay

The assay measures ligand-induced activation of the receptor CXCR2 in a stable cell line containing the recombinant human CXCR2 linked to a TEV protease site and a Gal4-VP16 transcription factor (Invitrogen). Ligand binding to the receptor results in the recruitment of arrestin proteins (tagged with protease) to the receptor and triggers the release of a tethered transcription factor. The transcription factor enters the nucleus and activates the transcription of the reporter gene. The ability of a compound to inhibit CXCR2 activation is indirectly assessed by measuring the reporter gene activity.

A vial of cryopreserved cells was removed from liquid nitrogen and rapidly thawed in a water bath at 37° C. with gentle agitation. The cell contents were collected and resuspended in Assay Medium at a density of ˜200,000 cells/ml. All test compounds were dissolved in DMSO at a concentration of 10 mM and then were serially diluted to generate a 10-point dose response curve into 384-well assay plate (Greiner 781090) using an Echo (Labcyte) concentration-response program (50 nl/well). The cell-free, un-stimulated and positive controls were loaded with 50 nl/well pure DMSO to ensure that the DMSO concentration was constant across the plate for all assays. Using Multi-drop Combi (Thermo) with standard cassette, 50 μl of Assay Medium was added to cell-free control wells in compound-containing assay plate; 50 μl of the cell suspension without hCXCL1 (a CXCR2 ligand) was added to un-stimulated control wells (10,000 cells per well); and 50 μl of the cell suspension with 80 nM hCXCL1 was added to the rest of wells of the assay plate (10,000 cells per well). The cells were incubated overnight at 37° C./5% CO₂·10 μl of 6× substrate mixture (LiveBLAzer™-FRET B/G substrate (CCF4-AM) Cat #K1096 from Invitrogen, Inc.) was added to each well using Multi-drop Combi (Thermo) with small-tube cassette and the plate was incubated at room temperature for 2 h in the dark. The plate was then read on EnVision using one excitation channel (409 nm) and two emission channels (460 nm and 530 nm).

The blue/green emission ratio (460 nm/530 nm) was calculated for each well by dividing the background-subtracted Blue emission values by the background-subtracted Green emission values. The dose response curve was based on sigmoidal dose-response model. All ratio data was normalized based upon the maximum emission ratio of positive control (hCXCL1) and minimum emission ratio of negative control (DMSO) on each plate.

As measured in this assay, Compound of Formula (A) gave a pIC50 of 9.2 against the CXCR2 receptor.

As measured in this assay, Compound of Formula (B) gave a pIC50 of 8.8 against the CXCR2 receptor.

As measured in this assay, Compound of Formula (C) gave a pIC50 of 9.0 against the CXCR2 receptor.

As measured in this assay, Compound of Formula (D) gave a pIC50 of 8.9 against the CXCR2 receptor.

As measured in this assay, Compound of Formula (E) gave a pIC50 of 8.8 against the CXCR2 receptor.

As measured in this assay, Compound of Formula (F) gave a pIC50 of 9.7 against the CXCR2 receptor.

As measured in this assay, Compound of Formula (G) gave a pIC50 of 9.0 against the CXCR2 receptor.

As measured in this assay, Compound of Formula (H) gave a pIC50 of 9.0 against the CXCR2 receptor.

As measured in this assay, Compound of Formula (I) gave a pIC50 of 9.4 against the CXCR2 receptor.

As measured in this assay, Compound of Formula (J) gave a pIC50 of 9.0 against the CXCR2 receptor.

d) Human Whole Blood Assay

Version i)

Human blood was obtained from healthy consenting adults by trained medical professionals by venipuncture in a 10 ml syringe containing 225 μI of 0.25 M EDTA and filling with 7 ml of blood (maintained at 37° C. throughout the CXCL1 stimulation).

The test compounds were dissolved in 100% DMSO to a concentration of 10 mM and diluted to 12× the assay concentration in 1.2% DMSO (final concentration DMSO of 0.1%).

Assay: 100 μI of whole blood was added to assay tubes or 96 well U-bottom clear plates containing 10 μI of 1.2% DMSO (assay concentration of 0.1%) or 10 μI of compound dissolved in 1.2% DMSO (assay concentration range of 0.1 μM to 10 μM), then incubated for 10 minutes at 37° C., followed by addition of 10 μI of 0.1% BSA in DPBS (negative controls) or 10 μI of 120 nM CXCL1 (GROα, Prepro Tech, Inc) dissolved in 0.1% BSA in DPBS (assay concentration of 10 nM which is the EC₈₀ for CXCL1) and incubated at 37° C. for an additional 10 min with gentle agitation before being placed on ice and assay terminated by adding 250 μI of CellFix or 250 μI of 3.7% v/v paraformaldehyde (BD Biosciences). After one minute, 50 μI of fixed blood was added to tubes containing 10 μI of anti CD11b-FITC (Beckman Coulter) and 5 μI of anti-human CD16-PE (DakoCytomation), gently mixed and total sample added to 500 μI of cold DPBS. Before being analyzed using an LSR flow cytometer (Becton-Dickinson) or FACS Canto II, cells were stained for 10 minutes at 4° C. with 10 μI of LDS-751 (Molecular Probes), a nucleic acid stain, allowing a cytometer threshold setting to exclude red blood cells. Analysis was done using Cell Quest Software or FACsdiva. The neutrophil population was determined by sequential gating; first gating on all granulocytes in the side scatter versus forward scatter plot, and then gating on the CD16+ (PE fluorescence) cells within the granulocyte population (CD16⁺ neutrophils, CD16⁻ eosinophils). The mean FITC fluorescence of the neutrophil population of each sample was assessed, as it directly relates to CD11b content, our biomarker for CXCR2 activation.

The mean fluorescence for each sample was determined. The positive control defined as fluorescence value of sample stimulated with 10 nM CXCL1 (no inhibitor). The negative control defined as fluorescence value of vehicle treated samples (no CXCL1, no inhibitor). The mean negative control value was subtracted from each sample. All samples were then normalized against the mean positive control.

As measured in this version of the assay, Compound of Formula (A) gave a pIC50 of 5.7 against the CXCR2 receptor.

As measured in this version of the assay, Compound of Formula (B) gave a pIC50 of 6.4 against the CXCR2 receptor.

As measured in this version of the assay, Compound of Formula (D) gave a pIC50 of 5.4 against the CXCR2 receptor.

Version ii)

Blood was withdrawn by venipuncture from consented adults and poured into a Sterilin tube containing an anti-coagulant Heparin (10 μL/mL of blood). All the test compounds were dissolved in DMSO to 4 mM and serial diluted across the plate, 1 in 3 to provide 10 point dose response curves. The compounds were then diluted 100 fold in -PBS [Phosphate buffered saline (without Calcium Chloride and Magnesium Chloride)] after which, 1 μL was dispensed in 96 U bottom Costar plates using an FX. This was done to reduce the final DMSO concentration to 0.25% and for compounds to be screened at 10 uM final assay concentration, after addition of blood.

10 μL of blood was transferred to the compound plate using a multi-channel pipette, gently tapped and incubated for 15 minutes, at 37° C. After 15 minutes, the stimulant GROα was diluted to 100 nM in 0.1% BSA (Albumin Bovine Serum)-PBS and 5 μL is dispensed across the whole plate for a final concentration of 33 nM. The plate was gently tapped and incubated further for 15 minutes at 37° C. The plate was placed on ice for 1 minute before addition of 10 μL of an antibody cocktail consisting of CD11b-FITC (40 ug/mL) purchased from BioLegend (address: Cambridge Bioscience, Munro House, Trafalgar Way, Bar Hill, Cambridge, UK) and CD16-PE purchased from BD Pharmingen (address: The Danby Building, Edmund Halley Road, Oxford Science Park, Oxford, UK) (stock concentration 100 tests, 2 mL stock volume is diluted 1 in 5). The plate was placed on ice for 1 hour in the dark. The cells were then fixed using 200 μL/well of 1× FACS (Flow Activated Cell Sorting) Lyse solution (Becton and Dickinson-BD) and on ice for 20 minutes in the dark. The plate was centrifuged at 1600 rpm for 5 minutes and re-suspended with 200 μL of ice cold PBS. This step was repeated twice and on the final step the plate was re-suspended with 50 μL of ice cold -PBS for flow Cytometric analysis.

Samples were run on a Becton and Dickinson (BD)-Acurri C6 Flow Cytometer using a HyperCyt sampling apparatus (IntelliCyt) with a flow rate 2 μL/sec. CD11 b upregulation is monitored in neutrophils and identified using a combination of both side scatter and CD16 expression.

As measured in this version of the assay, Compound of Formula (J) gave a pIC50 of 7.4 against the CXCR2 receptor.

As measured in this version of the assay, Compound of Formula (D) gave a pIC50 of 5.8 against the CXCR2 receptor. This value is similar to the pIC50 of 5.4 obtained for Compound of Formula D as measured in version ii) of this assay and therefore results from versions i) and ii) of assay d) are comparable.

e) GPCR Arrestin Assay

Certain compounds of the invention were also tested by DiscoveRX Corporation (42501 Albrae Street, Fremont, Calif. 94538, United States) in their GPCR Arrestin Assay to determine their activity against a panel of receptors including CXCR2 and CXCR1.

PathHunter CHO cell lines were expanded from freezer stocks according to standard procedures. Cells were seeded in a total volume of 20 μL into white walled, 384-well microplates and incubated at 37° C. for the appropriate time prior to testing.

The cells were pre-incubated with antagonist followed by agonist challenge at the EC80 concentration. Intermediate dilution of sample stocks was performed to generate 5× sample in assay buffer. 5 μL of 5× sample was added to cells and incubated at 37° C. or room temperature for 30 minutes. Vehicle concentration was 1%. 5 μL of 6× EC80 agonist in assay buffer was added to the cells and incubated at 37° C. or room temperature for 90 or 180 minutes.

Assay signal was generated through a single addition of 12.5 or 15 μL (50% v/v) of PathHunter Detection reagent cocktail, followed by one hour incubation at room temperature. Microplates were read following signal generation with a PerkinElmer Envision™ instrument for chemiluminescent signal detection.

Compound activity was analyzed using CBIS data analysis suite (ChemInnovation, Calif.). Percentage inhibition was calculated using the following formula: % Inhibition=100%×(1−(mean RLU of test sample−mean RLU of vehicle control)/(mean RLU of EC80 control−mean RLU of vehicle control)).

As measured in this assay, Compound of Formula (J) gave a pIC50 value of 8.3 against the CXCR2 receptor and 5.4 against the CXCR1 receptor. As measured in this assay, Compound of Formula (J) is 730 fold selective for CXCR2 over CXCR1.

As measured in this assay, Compound of Formula (D) gave a pIC50 value of 7.0 against the CXCR2 receptor and 5.6 against the CXCR1 receptor. As measured in this assay, Compound of Formula (D) is 29 fold selective for CXCR2 over CXCR1.

f) Effect of Oxaliplatin and Compound of Formula (A) in Colorectal Cancer Cell Line Proliferation Assay

Cell proliferation assay: Cells were seeded in 96-well plates (Costar) at different cell densities: HCT116, 500 cells per well; Caco-2, 1000 cells per well. Total volume of the medium was 90 μl per well. Cells were incubated for 24 hours at 37° C. and 5% CO₂ incubator.

Oxaliplatin and Compound of Formula (A) concentration series were further diluted with DPBS. The final concentrations of oxaliplatin in HCT116 cell line experiments were from 500 μM with three-fold dilution, fifteen concentrations and one control. The final concentrations of oxaliplatin in Caco-2 cell line experiments were from 55.5 μM with three-fold dilution, nine concentrations and one control. The final concentration of the Compound of Formula (A) was 8.5 μM.

5 μL of oxaliplatin together with 5 μl of vehicle or Compound of Formula (A) were added into the medium and incubated for an additional 72 hours at 37° C. and 5% CO₂ incubator. The cell proliferation rate was measured by adding 100 μl of CellTiter-Glo™ (Promega) according to the manufacturer's instructions. After 10 minutes of incubation, the cell lysates were transferred into OptiPlate-384 well, the luminescence was recorded by BioTek Synergy™ 4 Hybrid Microplate Reader with Gen5 software. All data points were generated in five replicates. Three independent experiments were performed.

Data Analysis: pIC50 values were calculated by software GraphPad Prism 5 using sigmoidal concentration-response (variable slope). All data were presented as the mean±standard error of the mean determined from three independent experiments.

The effects of Compound of Formula (A) alone (8.5 μM) on the basal proliferation of both Caco-2 and HCT116 cell lines were analysed by one-way ANOVA (GraphPad Prism 5) determined from three independent experiments. All data generated from both the entirety of the oxaliplatin concentration response curves and vehicle controls were included in these analyses to permit relevant statistical evaluations of the effect of the Compound of Formula (A) alone in the context of an established biological effect of oxaliplatin.

Results: Oxaliplatin demonstrated a concentration-dependent inhibition on cell proliferation in both Caco-2 and HCT116 cell lines, with pIC50 values of 5.923±0.047 and 6.049±0.016 respectively. In the presence of the Compound of Formula (A) at a concentration of 8.5 μM, these oxaliplatin inhibition of cell proliferation potencies were not affected with pIC50 values of 5.95±0.07and 6.05±0.02 respectively.

In Caco-2 and HCT116 cell lines; there were non-significant decreases of basal proliferation by the Compound of Formula (A) alone of 2.7±2.1% and 8.9±0.7% respectively. In addition these effects were less than the biologically relevant statistical differences observed in the respective oxaliplatin concentration response curves. Therefore the Compound of Formula (A) (8.5 μM) alone had no effect on the basal cell proliferation of the two cell lines.

g) Preclinical Gene Expression in Chronic Constriction Injury Rodents

The Chronic Constriction Injury (CCI) model of neuropathic pain involves unilateral loose ligation of a sciatic nerve with four ligatures. This results in the development of hyperalgesia, allodynia and spontaneous pain (ectopic discharges), in part caused by recruitment of inflammatory cells in the periphery and activation of microglia and astrocytes in the spinal cord. This model is believed to mimic some of the symptoms and aetiology of neuropathic pain observed in the clinic (Bennett and Xie, Pain 1988 33(1):87-107 (1988); Field et al., Pain 83(2):303-11 (1999). Early alterations of sensory endpoints in rodents have been seen in CCI models, resulting in increased sensitivity to thermal (Neuropharmacology, 79 (2014) 432-443; Pain, 101 (2003) 65-77) and mechanical stimuli (Neuropharmacology, 79 (2014) 432-443; Brain Research, 784(1998)154-162) using cold acetone stimuli and von Frey filament, respectively.

Male, Sprague Dawley rats (Harlan, UK. 200-250 grams) were housed in standard caging and laboratory conditions in groups of four, with free access to food (5CR4, Purina) and water (except during placement in the test box) on a 12/12 light/dark cycle. Enviromental enrichment was supplied from arrival day and changed on a Monday (castle), Wednesday (house) and Friday (tubes) to help prevent autotomy. Male, C57BL6 mice (Harlan, UK. age 6-7 wks) were housed in standard caging, laboratory conditions and enviromental enrichment in groups of five, with free access to food (5CR4, Purina) and water (except during placement in the test box) on a 12/12 light/dark cycle.

Behaviour Part:

Baseline testing: All animals underwent behavioural testing of mechanical allodynia prior to surgery in order to determine the baseline withdrawal thresholds. For the rat study, the average of the last two (out of three) baseline paw withdrawal thresholds (PWT) to stimulation with von-Frey hairs was taken as the baseline. For the mouse study, the baseline data was collected on two separate days (Day −1 and Day 0).

Surgery procedure: Under Isoflurane anaesthesia mixed with oxygen (3:1, 1 L/min), the left hind leg was shaved mid-thigh level and an incision made through the skin using a scalpel. The biceps femoris muscle layer was dissected by making an initial incision using a pair of sharp scissors, which was then widened using a pair of blunt scissors. The common sciatic nerve was exposed and 3 (for mice) or 4 (for rats) loose ligatures of prolene (for mice) or chromic gut (SMI) (for rats) were tied around the sciatic nerve with 1 mm spacing between each. The nerve was then returned below the muscle layer and the wound closed using absorbable sutures (Vicryl). Sham animals underwent the same procedure except that the nerve was left un-ligated.

Behavioural testing was started 3 days post-surgery with mechanical response thresholds taken on Day 3 and Day 7. Tissues from the DRG and sciatic nerve were collected on Day 1, Day 3 and Day 7 following CCI surgery treatment. A separate group of surgically prepared animals was used for tissue collection on Days 1 and 3 and behavioral assessment on Day 3. All tissues were snap frozen by placement in appropriate sized, labelled tube and placed into liqiud nitrogen.

Static Mechanical (Tactile) Allodynia:

Rats: The animals were placed on an elevated mesh-bottom platform with a 0.5 cm² grid and an inverted plexiglass container was placed overeach animal. Testing was performed after an initial 15-20 minute acclimatisation/habituation period. Measurement of withdrawal threshold was achieved using calibrated (force; g) von-Frey monofilaments (Touch-Test Sensory Evaluator; Scientific Marketing Associates) applied to the plantar surface of the hindpaw for approximately 8 seconds with enough force to cause a slight bend of the filament. Withdrawal threshold was determined by increasing and decreasing stimulus intensity and estimated using the Dixon's up-down method [Dixon, Annu. Rev. Pharmacol. Toxicol., 20, 441-62 (1980); Chaplan et al., J. Neurosci. Methods, July, 53(1), 55-63 (1994)]. Only immediate sharp withdrawal responses from the stimulus (or flinching) were considered to represent a positive response.

Mice: Static mechanical alodynia was assessed by measurement of withdrawal threshold using calibrated (force; g) von-Frey monofilaments (Touch-Test Sensory Evaluator; Scientific Marketing Associates) applied to the plantar surface of the hindpaw. The animals were placed in individual Perspex boxes on a raised metal mesh for 30-40 min before the test. A series of graduated von Frey hairs (0.07, 0.16, 0.4, 0.6 and 1 g) were applied in sequence with a protocol of 1 second on 1 second off repeated 10 times. Each hair was applied perpendicularly to the center of the ventral surface of the paw until it slightly bends. The force applied to the hind-paw of the animal to induce 5 responses out of 10 trails will be recorded as PWT. Only immediate sharp withdrawal responses from the stimulus (or flinching) were considered to represent a positive response. PWT will be assessed on two consecutive days (Day −1 and Day 0) re-assessed on Day 3 and Day 7, following surgery to monitor the development of the mechanical allodynia.

Statistic analysis: Behaviour Data were analyzed by Graphpad Prism using regular two-way ANOVA with ‘treatment’ as a between subjects effect and ‘day’ as a within subjects effect. Post-hoc analysis was performed using Bonferroni method. A p value of <0.05 was considered to be statistically significant.

mRNA expression: Quant Studio Open Array was used to assess the effects of CCI on the expression levels of NPY (Neuropeptide Y), CXCR2, and its cognant ligand CXCL1 following the tactile behavioural endpoint.

The sciatic nerves of rat and mouse were dissected out and snap frozen in liquid nitrogen. The tissues were stored frozen at −80° C. or on dry ice until processed for RNA analysis of CXCR2 and CXCL1. ActB, GAPDH, HPRT1 and MAPK6 were used as internal assay controls and standards, and NPY was used as control for the CCI.

The following genes were measured using TaqMan® Q-PCR. Assays were used to make the preamplification pool.

Rat Specific Assays

| Gene | Taqman Assay |
|---|---|
| symbol | ID |
| Act B | Rn00667869_m1 |
| CXCL1 | Rn00578225_m1 |
| CXCR2 | Rn00567841_m1 |
| GAPDH | Rn01775763_g1 |
| HPRT1 | Rn01527840_m1 |
| MAPK6 | Rn00581152_m1 |
| NPY | Rn01410145_m1 |

Mouse Specific Assays

| Gene | |
|---|---|
| symbol | Taqman Assay ID |
| Act B | Mm01205647_g1 |
| CXCL1 | Mm04207460_m1 |
| CXCR2 | Mm00438258_m1 |
| GAPDH | Mm99999915_g1 |
| HPRT1 | Mm01545399_m1 |
| MAPK6 | Mm00727050_s1 |
| NPY | Mm03048253_m1 |

Total RNA Isolation: For each tissue sample, one RNA sample was prepared by homogenizing the tissue in MagNA Pure LC lysis buffer (Roche) in Green Bead tubes (Roche) on the Roche MagnaLyser according to the manufacturers' instructions and the total RNA isolation was carried out using the Roche MagnaPure RNA isolation system (Roche) according to instructions in the manufacturers handbook. Samples were eluted in 50 μL aliquots and stored at −80° C. until required.

RNA Quantification and Assessment of Integrity: Each total RNA sample was quantified on a LifeTechnologies Qubit according to the manufacturer's handbook. Integrity of a representative sample of total RNA samples was assessed on an Agilent 2100 Bioanalyser, using an Agilent Pico kit (Agilent Technologies), according to the manufacturer's handbook. RNA was considered of acceptable quality if the RIN (RNA integrity number) was 6 or more.

First Strand cDNA Synthesis: First strand cDNA was synthesized from 50 ng of each total RNA using the Superscript Vilo kit (Life Technologies). Each RNA was made up to 11 μL with RNase free water and 14 μL master mix added to each RNA sample.

Mastermix was made up based on the table below:

| Component | Volume |
|---|---|
| 5x Vilo RT buffer | 5 μL |
| 10x Vilo Enzyme mix | 2.5 μL |
| Nuclease - free water | 6.5 μL |
| Final volume RT master mix | 14 μL |

The samples were incubated at 25° C. for 10 mins, 42° C. for 60 mins followed by 85° C. for 5 minutes in a DNA Thermocycler. Following synthesis, the cDNA was stored at −20° C. until ready for use.

PreAmplification: A preamplification pool of Taqman Assays (Life Technologies) was made by mixing 10 μL of each ×20 assay together (16 assays in total: 4 genes of interest (rat), 4 Housekeepers (rat), 4 genes of interest (mouse) and 4 Housekeepers (mouse)) and diluting to ×0.2 (by addition of 840 μL TE buffer (Life Technologies))

Each 25 μL cDNA was amplified by incubation with the following:

| Reagent | Volume |
|---|---|
| 2x PreAmp MM | 25 μL |
| 0.2X Pooled assay mix | 12.5 μL |

The following cycling conditions were used:

| | Stage | Repeats | Temperature | Time |
|---|---|---|---|---|
| Enzyme activation(hold) | 1 | 1 | 95° C. | 10 mins |
| PCR (cycle) | 2 | 14 | 95° C. | 30 sec |
| | | | 60° C. | 4 mins |
| Enzyme inactivation | | | 99° C. | 10 mins |

The preamplification products were stored at −20° C. until ready for use and then diluted 1:10 with 0.1×TE (pH 8.0) before running on TaqMan Open Array.

TaqMan® Real-Time Quantitative PCR: QuantStudio open arrays were set up and run according to LifeTechnologies guidelines. 2× TaqMan® OpenArray® Real-Time PCR Master Mix (Life Technologies; cat: 4462164) were mixed and the following components combined:

| Component | Volume for 1 sample |
|---|---|
| 2x OpenArray master mix | 2.5 μL |
| water | 1.3 μL |

1.2 μL of each preamplified cDNA was mixed with 3.8 μL of Open Array mastermix and placed in an Accufill 384-well plate for dispensing onto the Open Array by the Accufill™ system. The OpenArray Sample Tracker Software was used to track the samples. The Open Array was run on the QuantStudio and was analyzed and quality controlled on the QuantStudio and the resulting files exported to ArrayStudio for data analysis.

Assessment of the expression CXCR2, CXCL1 and NPY in the sciatic nerve of mice and rats by QuantStudio Open Array: Analysis in ArrayStudio: QuantStudio.txt files were annotated with OpenArray barcode, deleted all rows with empty Target Name values and sorted by well into a format for ArrayStudio. Individual Open Array.txt files were uploaded into Array Studio. “No Reverse transcription”, “no template controls” and “water blank” data were inspected, but removed from the final analysis. A principal components analysis (PCA) was performed on the four housekeeping genes. Outlier data were removed after the first PCA run. After adjusting for the effects of covariance through the housekeeping genes, the model was used to estimate the mean log 2(Abundance) for each gene and treatment. Full linear model of analysis and factor for analysis was selected and run. This generated a summary table of normalized data, a summary table of outliers removed, a coefficient for each gene and P-Value indicating whether the gene was improved by normalisation. These data were analysed by 3 way principal component analysis to check that biological replicate samples were clustering together. The data were analysed by 1-way ANOVA within each model and species to generate a fold difference value between different treatments at each timepoint. Bayesian posterior probabilities that the mean difference does not contain zero were calculated. A probability greater than 0.95 was taken to indicate that the mean difference was significantly different from zero. For the CCI model, ipsilateral and contralateral samples of like tissues were compared.

Results: There were no differences in baseline paw withdrawal thresholds (PWT) across the groups between rat and mouse experiments involving CCI when looking at tissue expression.

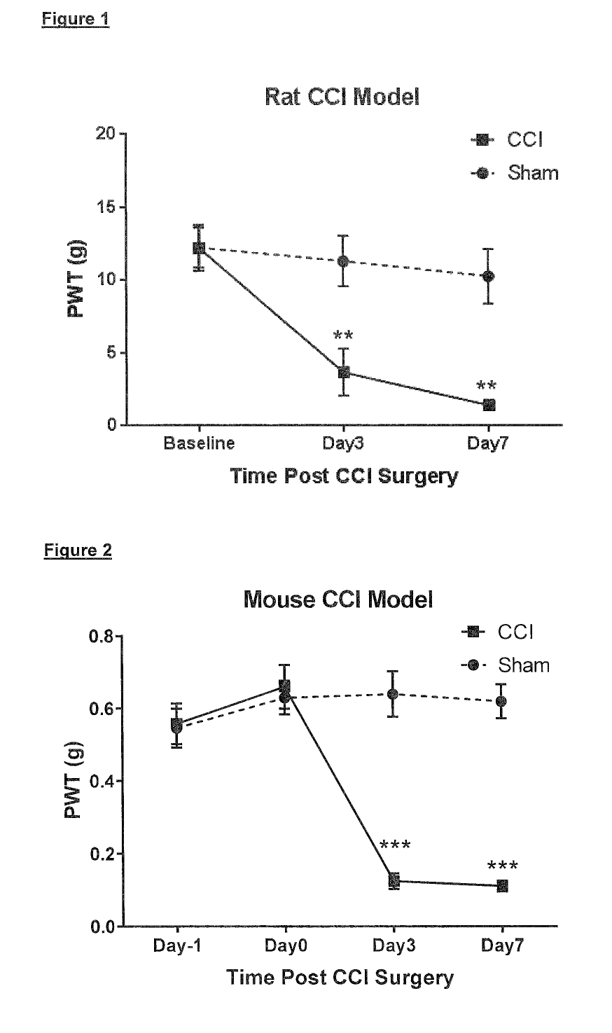

CCI induced a significant mechanical allodynia phenotype as reflected by significant reductions in PWTs in both rats (FIG. 1) and mice (FIG. 2). In rats, the PWT of the CCI group significantly decreased on Day 3 and Day 7 after surgery (FIG. 1) when compared with baseline values and the Sham group at these time points. (Values are mean±SEM. **P<0.01, indicates significance compared to the Sham group at the same time point, 2-way ANOVA. Baseline: n=20/group; Day 3: n=15/group; Day 7: n=10/group). Similarly mice developed mechanical allodynia following the chronic constriction injury. The PWT of the CCI group significantly decreased on Day 3 and Day 7 after surgery (FIG. 2). (Values are mean±SEM. ***P<0.001, indicates significance compared to the Sham group at the same time point, 2-way ANOVA. Day-1: n=20 in Sham group, n=21 in CCI group; Day 0: n=20 in Sham group, n=10 in CCI group; Day 3: n=15 in Sham group, n=16 in CCI group; Day 7: n=10 in Sham group, n=11 in CCI group).

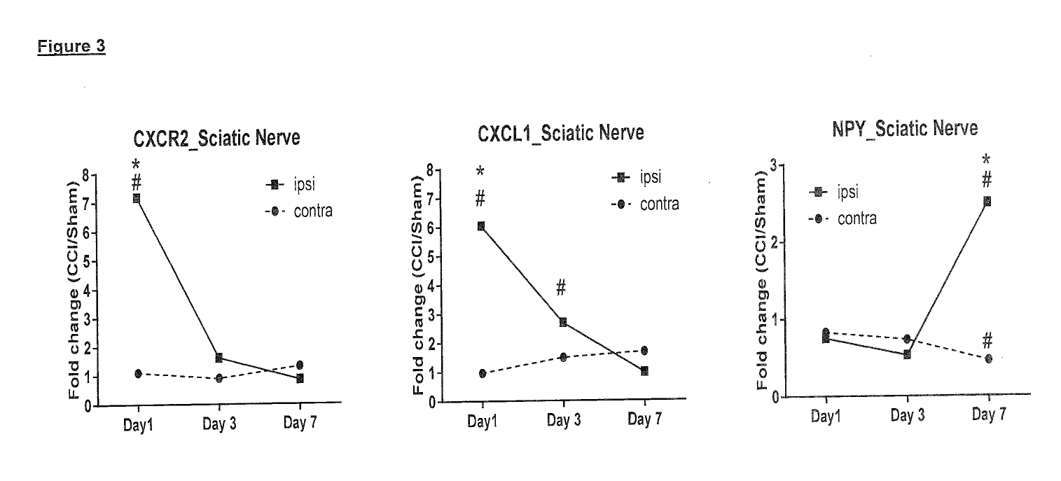

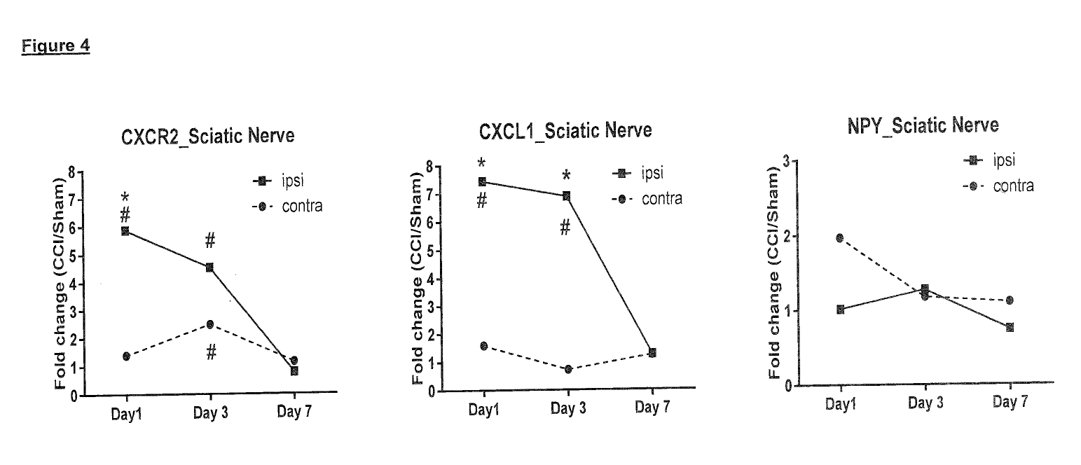

It can be seen from FIG. 3 (rat) and FIG. 4 (mice) that the expression profiles of CXCR2 and CXCL1 in both rats and mice were significantly elevated at the site of the CCI (ipsilateral; ipsi) in the sciatic nerve at Day 1 post CCI induction as evidenced by a statistically significant increase in fold change compared to sham treated animals. The expression profile of the neurosensory peptide, Neuropeptide-Y (NPY), was only significantly elevated in rat at the site of the CCI (ipsilateral; ipsi) in the sciatic nerve at Day 3 post CCI induction when compared with sham treated animals. It can be seen from FIG. 3 that compared with the sham-treated animals or the contralateral tissue, CXCR2, and its ligand CXCL1 mRNA were significantly increased in the ipsilateral sciatic nerve at an early stage after the CCI surgery that waned by Day 7. Neuropeptide-Y (NPY) mRNA was statistically different compared to sham animals and the contralateral tissue on Day 7. Values are shown as means. # indicates significant difference between CCI and Sham rats at the same time point, * indicates significant difference between contralateral and ipsilateral sciatic nerve at the same time point, n=5/group at each time point, assessed by Bayesian posterior probability>0.95. ipsi=ipsilateral; contra=contralateral. It can be seen from FIG. 4 (mice) that compared with the sham animal or the contralateral tissue, CXCR2, and its ligand CXCL1 mRNA was significantly increased in the ipsilateral sciatic nerve at an early stage after the CCI surgery that waned by Day 7. Neuropeptide-Y (NPY) mRNA was not statistically different compared to sham animals or the contralateral tissue at any of the time points that measurements were made. Values are shown as mean. # indicates significant difference between CCI and Sham mice at the same time point, * indicates significant difference between contralateral and ipsilateral sciatic nerve at the same time point, n=5 at each time point, assessed by Bayesian posterior probability>0.95.ipsi=ipsilateral; contra=contralateral.

Summary: The CCI procedure in both rodent species caused the development of robust mechanical allodynia. In both species, enhanced CXCR2 and CXCL1 mRNA expression was evident early in the temporal assessment of mechanical alloydnia. There were also changes in sensory Neuropeptide Y expression in the rat. This profile in the rat would be consistent with the known sensitivity of the CXCR2 system and sensory neuropeptides in the CCI model and allows this methodology to be used for detecting these proteins in oxaliplatin models.

h) Preclinical Gene Expression in Oxaliplatin-Induced Acute Sensory Injury Rodents

Adult male Sprague-Dawley rats (230-300 g) were housed in perspex cages in groups of 3˜5 in a controlled environment of constant temperature and moisture (Temperature: 21±1° C., light: dark cycle of 12:12 hours) with food and water available ad libitum. Adult male C57 BL6 mice (21-30 g) were housed in perspex cages in groups of 3˜5 in a controlled environment of constant temperature and moisture (Temperature: 21±1° C., light: dark cycle of 12:12 hours) with food and water available ad libitum. The animals are allowed to recover from transportation for at least one week before commencing experiments.

Baseline testing: All animals underwent behavioural testing of mechanical allodynia prior to injection in order to determine baseline withdrawal thresholds. Baseline PWT to stimulation with von-Frey hairs were taken on the last two days (Day −1 and Day 0).

Neuropathy induction: A 2 mg/ml solution of oxaliplatin in 5% dextrose was made. For rats, a single dose of oxaliplatin was administrated via the intra-peritoneal route at 12 mg/kg at a volume of 6 ml/kg on Day 0. The vehicle control group received 5% dextrose solutions at 6 ml/kg, ip. For mice, a single dose of oxaliplatin was administrated via the intra-peritoneal route at 15 mg/kg at a volume of 7.5 ml/kg on Day 0. The vehicle control group received 5% dextrose solutions at 7.5 ml/kg, ip.

Behaviour Part:

The development of mechanical allodynia was determined by measuring PWT following administration of a single dose of either vehicle or oxaliplatin. PWT was determined on two consecutive days (Day −1 and Day 0) and then re-assessed on Day 1, Day 3 and/or Day 7.

Rats: The animals were placed in individual perspex boxes on a raised metal mesh for at least 40 minutes before the test. Starting from the filament of lowest force (about 1 g), each filament was applied perpendicularly to the centre of the ventral surface of the paw until slightly bent for 6 seconds. If the animal withdrew or lifted the paw upon stimulation, then a hair with force immediately lower than that tested was used. If no response was observed, then a hair with force immediately higher was tested. The lowest amount of force required to induce reliable responses (positive in 3 out of 5 trials) was recorded as the value of PWT.

Mice: The animals were placed in individual Perspex boxes on a raised metal mesh for 30-40 min before the test. A series of graduated von Frey hairs (0.07, 0.16, 0.4, 0.6 and 1g) were applied in sequence with a protocol of 1 sec on 1 sec off repeated 10 times. Each hair was applied perpendicularly to the centre of the ventral surface of the paw until it slightly bent. The force applied to the hind-paw of the animal to induce 5 responses out of 10 trials was recorded as PWT.

Tissue sampling: At the end of the experiments, the animals were anaesthetized with a mixed gas comprised of isoflurane+oxygen and killed by decapitation. A segment of sciatic nerve from each side, were surgically excised, weighed on an analysis balance and recorded. The sciatic nerves from each side were placed in 2 ml Corning vials separately. The tissues were snap-frozen in liquid nitrogen, then stored at −80 degree.

Statistic analysis: Behavioural data were analyzed by Graphpad Prism using regular two-way ANOVA with ‘treatment’ as a between subjects effect and ‘day’ as a within subjects effect. Post-hoc analysis was performed using Bonferroni method. A p value of <0.05 was considered to be statistically significant.

Gene expression part: Sciatic nerves of the rodents were dissected out and snap frozen in liquid nitrogen. The tissues were stored frozen at −80° C. or on dry ice until processed for RNA analysis of CXCR2 and CXCL1. ActB, GAPDH, HPRT1 and MAPK6 were used as internal assay controls and standards, and NPY was used as the control for the oxaliplatin induced injury.

The following genes were measured using TaqMan® Q-PCR. Assays were used to make the preamplification pool.

Rat Specific Assays

| Gene | Taqman Assay |
|---|---|
| symbol | ID |
| Act B | Rn00667869_m1 |
| CXCL1 | Rn00578225_m1 |
| CXCR2 | Rn00567841_m1 |
| GAPDH | Rn01775763_g1 |
| HPRT1 | Rn01527840_m1 |
| MAPK6 | Rn00581152_m1 |
| NPY | Rn01410145_m1 |

Mouse Specific Assays

| Gene | |
|---|---|
| symbol | Taqman Assay ID |
| Act B | Mm01205647_g1 |
| CXCL1 | Mm04207460_m1 |
| CXCR2 | Mm00438258_m1 |
| GAPDH | Mm99999915_g1 |
| HPRT1 | Mm01545399_m1 |
| MAPK6 | Mm00727050_s1 |
| NPY | Mm03048253_m1 |

Total RNA Isolation: For each tissue sample, one RNA sample was prepared by homogenizing the tissue in MagNA Pure LC lysis buffer (Roche) in Green Bead tubes (Roche) on the Roche MagnaLyser according to the manufacturer's instructions and the total RNA isolation was carried out using the Roche MagnaPure RNA isolation system (Roche) also according to manufacture's instructions. Samples were eluted in 50 μL aliquots and stored at −80° C. until required.

RNA Quantification and Assessment of Integrity: Each total RNA sample was quantified on a LifeTechnologies Qubit according to the manufacturer's instructions. The integrity of a representative sample of total RNA samples was assessed on an Agilent 2100 Bioanalyser, using an Agilent Pico kit (Agilent Technologies), according to the manufacturer's instructions. RNA was considered of acceptable quality if the RIN was 6 or more.

First Strand cDNA Synthesis: First strand cDNA was synthesised from 50 ng of each total RNA using the Superscript Vilo kit (Life Technologies). Each RNA was made up to 11 μL with RNAse free water and 14 μL master mix added to each RNA sample. Mastermix was made up based on the table below:

| Component | Volume |
|---|---|
| 5x Vilo RT buffer | 5 μL |
| 10x Vilo Enzyme mix | 2.5 μL |
| Nuclease - free water | 6.5 μL |
| Final volume RT master mix | 14 μL |

The samples were incubated at 25° C. for 10 mins, 42° C. for 60 mins followed by 85° C. for 5 minutes in a DNA Thermocycler. Following synthesis, the cDNA was stored at -20° C. until ready for use.

PreAmplification: A preamplification pool of Taqman Assays (Life Technologies) were made by mixing 10 μL of each ×20 assay together [16 assays in total: 4 genes of interest (rat), 4 Housekeeping genes (rat)] and diluting to ×0.2 [by addition of 840 μL TE buffer (Life Technologies)]. Each 25 μL cDNA aliquot was amplified by incubation with the following:

| Reagent | Volume |
|---|---|
| 2x PreAmp MM | 25 μL |
| 0.2X Pooled assay mix | 12.5 μL |

The following cycling conditions were used:

| | Stage | Repeats | Temperature | Time |
|---|---|---|---|---|
| Enzyme activation(hold) | 1 | 1 | 95° C. | 10 mins |
| PCR (cycle) | 2 | 14 | 95° C. | 30 sec |
| | | | 60° C. | 4 mins |
| Enzyme inactivation | | | 99° C. | 10 mins |

The preamplification products were stored at −20° C. until ready for use and diluted 1:10 with 0.1× TE (pH 8.0) before running on TaqMan Open Array.

TaqMan® Real-Time Quantitative PCR: QuantStudio open arrays were set up and run according to LifeTechnologies guidelines. 2× TaqMan® OpenArray® Real-Time PCR Master Mix (Life Technologies; cat: 4462164) werewas mixed and the following components combined:

| Component | Volume for 1 sample |
|---|---|
| 2x OpenArray master mix | 2.5 μL |
| water | 1.3 μL |

1.2 μL of each preamplified cDNA was mixed with 3.8 μL of Open Array mastermix and placed in an Accufill 384-well plate for dispensing onto the Open Array by the Accufill™ system. The OpenArray Sample Tracker Software was used to track the samples. The Open Array was run on the QuantStudio and were analyzed and quality controlled on the QuantStudio and the resulting files exported to ArrayStudio for data analysis.

Assessment of the Expression CXCR2, CXCL1, NPY, and CGRP in the Sciatic Nerve of Rats by QuantStudio Open Array.

Analysis in ArrayStudio: QuantStudio.txt files were annotated with OpenArray barcode, deleted all rows with empty Target Name values and sorted by well into a format for ArrayStudio. Individual Open Array.txt files were uploaded into Array Studio. “No Reverse transcription”, “no template controls” and “water blank” data were inspected, but removed from the final analysis. A principal components analysis (PCA) was performed on the four housekeeping genes. The outlier data removed after the first PCA run. After adjusting for the effects of covariance through the housekeeping genes, the model was used to estimate the mean log 2(Abundance) for each gene and treatment. Full linear model of analysis and factor for analysis was selected and run. This generated a summary table of normalized data, a summary table of outliers removed, a coefficient for each gene and P-Value indicating whether the gene was improved by normalisation. These data were analysed by 3 way principal component analysis to check that biological replicate samples were clustering together. The data were analysed by 1-way ANOVA within each model and species to generate a fold difference value between different treatments at each timepoint. Bayesian posterior probabilities that the mean difference does not contain zero were calculated. Bayesian posterior probabilities that the mean difference does not contain zero were calculated. If the probability is greater than 0.95, that means the mean difference is significantly different from zero. Each tissue was compared separately.

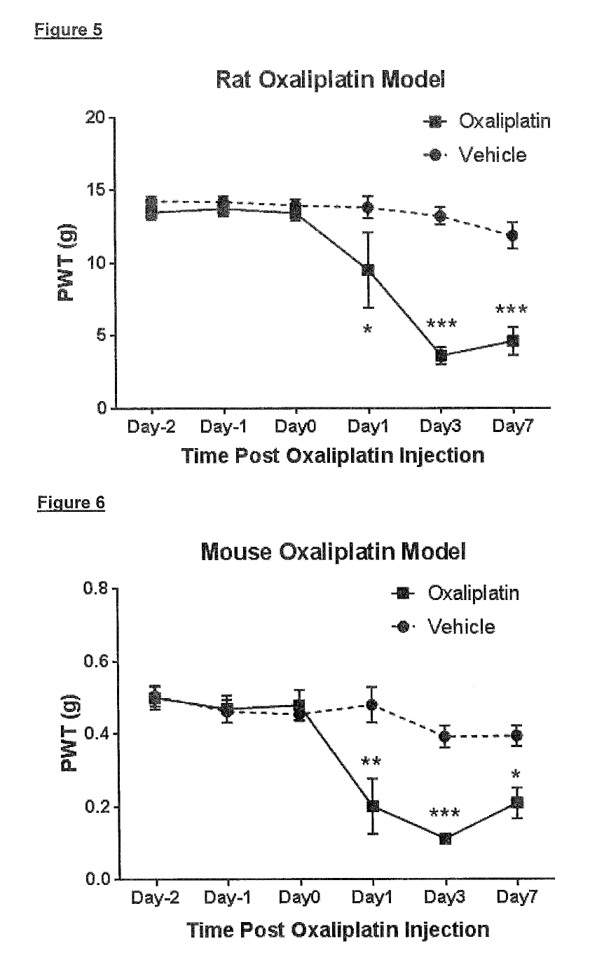

Results: There were no differences in baseline PWTs across the groups between rat and mouse experiments involving oxaliplatin. As can be seen from FIG. 5 (rats) and FIG. 6 (mice), oxaliplatin treatment caused a significant mechanical allodynia in both rodent species as represented by significant decreases in PWTs from Day 1 that was maintained through to Day 7. It can be seen in FIG. 5 that rats developed the mechanical allodynia following a single dose of oxaliplatin. The PWT of the oxaliplatin group statistically decreased at Day 1, 3 and 7. Values are mean±SEM. *P<0.05, ***P<0.001, indicates significance compared to the Vehicle group at the same time point, 2-way ANOVA. Day −2, Day −1, Day 0: n=20/group; Day1: n=5/group; Day 3: n=15/group; Day 7: n=10/group. It can be seen in FIG. 6, that mice developed the mechanical allodynia following a single dose of oxaliplatin. The PWT of the oxaliplatin group statistically decreased at Day 1, 3 and 7. Values are mean±SEM. *P<0.05, **P<0.01, ***P<0.001, indicates significance compared to the Vehicle group at the same time point, 2-way ANOVA. Day −2, Day −1, Day 0: n=20/group; Day 1: n=5/group; Day 3: n=15/group; Day 7: n=10/group.

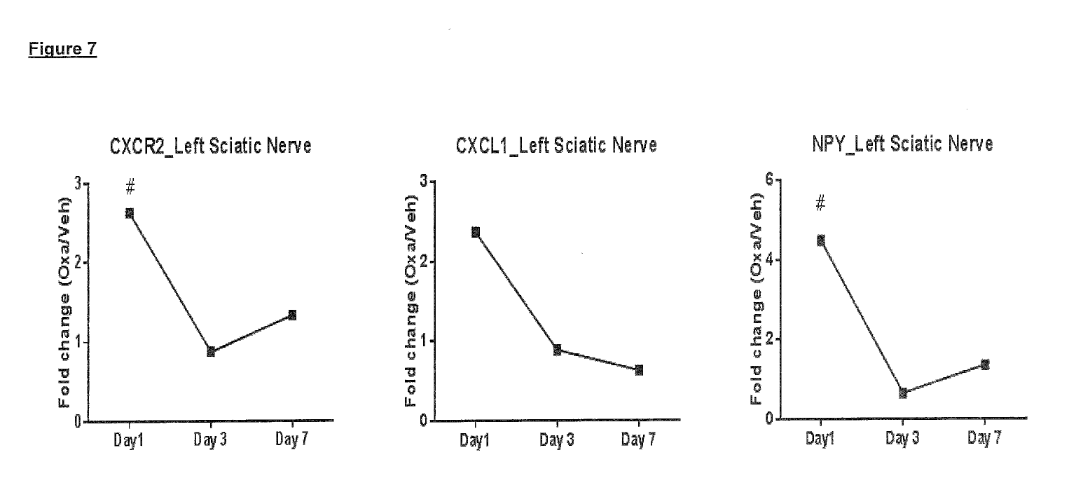

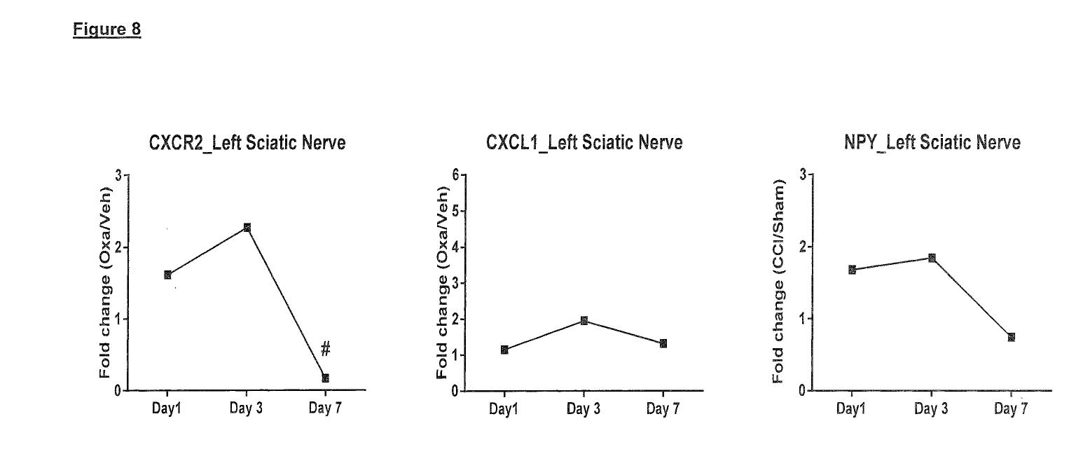

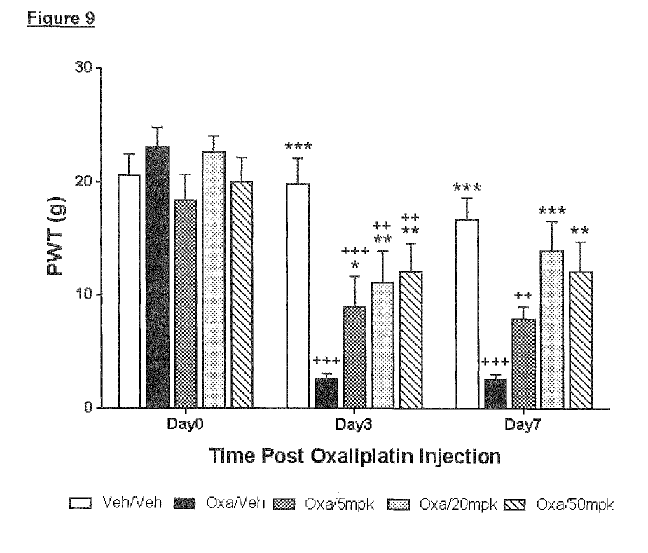

As can be seen from FIG. 7 (rats) and FIG. 8 (mice), the expression profile of CXCR2 and NPY mRNA after oxaliplatin treatment was significantly elevated in the rat left sciatic nerve at Day 1 post oxaliplatin treatment but not evident in the mouse. The expression profile of CXCL1 mRNA after oxaliplatin treatment in the left sciatic nerve was not statistically different post oxaliplatin treatment in both species. It can be seen from FIG. 7, that in the left sciatic nerve, CXCR2, and its ligand CXCL1 mRNA was highly increased at an early stage (Day1) after the oxaliplatin injection, as was NPY mRNA. Values are shown as mean. # indicates significant difference between oxaliplatin and vehicle group at the same time point, n=10 at each time point, assessed by Bayesian posterior probability>0.95. It can be seen from FIG. 8 that in the left sciatic nerve, neither CXCR2, or its ligand CXCL1 mRNA nor the NPY mRNA was increased with significance within 7 days post oxaliplatin injection. Values are shown as mean. # indicates significant difference between oxaliplatin and vehicle group at the same time point, n=10 at each time point, assessed by Bayesian posterior probability >0.95.

Summary: Both rats and mice developed robust mechanical allodynia following single oxaliplatin dosing. However, early statistically significant changes in CXCR2 and NPY mRNA expression (increased in the left sciatic nerve) were only apparent in the rat. Therefore the rat was selected as the primary species with the potential to explore the effects of pharmacological inhibition of the CXCR2 receptor on oxaliplatin induced mechanical alloydnia.

i) Preclinical CXCR2 Antagonist Effect on Oxaliplatin-Induced Acute Sensory Injury Rat

Animals: 60 Adult male Sprague-Dawley rats (Charles River, UK. 230-300 g) were used for this study. The animals were housed in perspex cages in groups of 3-5 in a controlled environment of constant temperature and moisture (Temperature: 21±1° C., light: dark cycle of 12:12 hours) with food and water available ad libitum. They were allowed to recover from transportation for at least one week before commencing experiments.

Compound of Formula (A) was prepared as 0.5 mg/ml, 2 mg/ml and 5 mg/ml suspensions (aiming for 5 mg/kg, 20 mg/kg and 50 mg/kg dosing respectively) by wet grinding and then sonicating in 1% methylcellulose (MC). The formulation was prepared weekly and stored at 4° C. protected from light. The dose volume was 10 ml/kg. The compound was stirred continuously throughout the dosing period. Methylcellulose (Sigma) was made up in water at 1% (1 g per 100 ml water) and left on a stirrer until completely dissolved. Vehicle treated animals were dosed b.i.d. with 1% MC at 10 ml/kg p.o.

Oxaliplatin (Tocris bioscience) was prepared as a 2 mg/ml solution (aiming for 12 mg/kg) by sonicating in 5% dextrose (Baxter). The formulation was prepared fresh on the day of dosing. The dose volume was 6 ml/kg.

Study scheme: The Study Scheme is shown in Table 1. Oxaliplatin was injected on Day 0 once, and three doses of Compound of Formula (A) were given twice daily from Day −1 to Day 6. PWT was tested on Day −2, Day −1, Day 0, Day 3 and Day 7.

TABLE 1

| Time | Day −2 | Day −1 | Day 0 | Day 1 | Day 2 | Day 3 | Day 4 | Day 5 | Day 6 | Day 7 |
|---|---|---|---|---|---|---|---|---|---|---|
| 8.30- | PWT | PWT | PWT | | | PWT | | | | PWT |
| 10.00 | | | | | | | | | | |
| 10.00- | | 1st | 1st | 1st | 1st | 1st | 1st | 1st | 1st | End |
| 10.30 | | dose | dose | dose | dose | dose | dose | dose | dose | of Life |
| 11.00- | | | oxaliplatin | | | | | | | |
| 11.30 | | | injection | | | | | | | |
| 1700- | | 2nd | 2nd | 2nd | 2nd | 2nd | 2nd | 2nd | 2nd | |
| 1700 | | dose | dose | dose | dose | dose | dose | dose | dose | |

The following five groups were studied:

| | |
|---|---|
| Group 1 | Veh/Veh, 5% dextrose on Day 0, 1% MC from Day −1 to Day 6 |
| Group 2 | Oxa/Veh, 12 mg/kg oxaliplatin on Day 0, 1% MC from Day −1 |
| | to Day 6 |
| Group 3 | Oxa/5 mpk, 12 mg/kg oxaliplatin on Day 0, 5 mg/kg Compound |
| | of Formula (A) from Day −1 to Day 6 |
| Group 4 | Oxa/20 mpk, 12 mg/kg oxaliplatin on Day 0, 20 mg/kg |
| | Compound of Formula (A) from Day −1 to Day 6 |
| Group 5 | Oxa/50 mpk, 12 mg/kg oxaliplatin on Day 0, 50 mg/kg |
| | Compound of Formula (A) from Day −1 to Day 6 |

Induction of chemotherapy-induced neuropathy: A single dose of oxaliplatin was injected intraperitoneally (i.p.) at 12 mg/kg at a volume of 6 ml/kg. oxaliplatin was dissolved in 5% dextrose to 2 mg/ml before use. The development of neuropathic pain, characterised by significant mechanical allodynia, was monitored using a series of graduated von Frey hairs applied to the hind-paws to trigger a withdrawal response (PWT, see below). The vehicle control group received 5% dextrose solutions at 6 ml/kg, i.p. Animals were injected in the same order as which they were tested.

The animals were placed in individual perspex boxes on a raised metal mesh for at least 40 minutes before the test. Starting from the filament of lowest force (approximately 1 g), each filament was applied perpendicularly to the centre of the ventral surface of the paw until slightly bent for 6 seconds. If the animal withdrew or lifted the paw upon stimulation, then a hair with force immediately lower than that tested was used. If no response was observed, then a hair with force immediately higher was tested. The lowest amount of force required to induce reliable responses (positive in 3 out of 5 trials) was recorded as the value of PWT. PWT was assessed on three consecutive days (Day −2, Day −1 and Day 0) and re-assessed on Day 3 and Day 7, following administration of a single dose of either vehicle or oxaliplatin to monitor the development of mechanical allodynia. Day −2 and Day −1 were considered the baseline prior to oxaliplatin dosing and Day 0 was the reading taken on the day of oxaliplatin injection.

Statistic analysis: The behaviour data was analyzed by two-way repeated measures ANOVA with ‘treatment’ as a between subjects effect, and ‘day’ as a within subjects effects. Post-hoc analysis was performed using planned pair-wise comparison [InVivoStat; Clark et al., J. Psychopharmacology, 26(8) 1136-1142 (2012)].

Results: Injection of oxaliplatin resulted in the development of mechanical allodynia which was measured using von-Frey assessment of PWT. Prophylactic treatment with Compound of Formula (A) (starting on Day −1) prior to oxaliplatin resulted in prevention of the development of oxaliplatin induced mechanical allodynia. This was apparent at all dose levels on Day 3 post Oxaliplatin treatment and at 20 and 50 mg/kg bid po on Day 7 post oxaliplatin treatment. As FIG. 9 shows, a bolus injection of oxaliplatin resulted in the development of a marked mechanical allodynia. The PWT was significantly reduced compared to the Veh/Veh group on Day 3 and Day 7. The prophylactic Compound of Formula (A) dosing inhibited the mechanical allodynia established by the oxaliplatin treatment, PWT significantly increased compared to the Oxa/Veh group. Values are mean±SEM. *P<0.05, **P<0.01 and ***P<0.001 indicates significance compared to the Oxa/Veh group, ++p<0.01 and +++p<0.001 indicates significance compared to the Veh/Veh group, 2-way ANOVA. Oxa=oxaliplatin; Veh=vehicle; 5, 20 and 50 mpk=dose level of Compound of Formula (A) as mg/kg bid po.

Summary: A single bolus injection of oxaliplatin resulted in the development of mechanical allodynia in rats. Prophylactic treatment with Compound of Formula (A) attenuated the development of mechanical allodynia. At Day 3, all three dose levels of Compound of Formula (A) (5, 20 and 50 mg/kg p.o.b.i.d) had reduced the mechanical allodynia compared with the oxaliplatin and vehicle group. By Day 7, there was still present a robust reduction of the mechanical allodynia in animals receiving Compound of Formula (A) at 20 and 50 mg/kg p.o.b.i.d only.

j) Preclinical CXCR2 Antagonist Effect on Repeat Dosing Oxaliplatin Induced Sensory Injury Rat

Chemotherapy treatment is well established to cause significant decreases in the conduction potential of nerves in humans and in rodents [Renn et al., Mol. Pain, 26; 7:29, (2011)]. Therefore to compliment investigations on the behavioural hypersensitivity induced by oxaliplatin, eletrophysiological studies of the sciatic nerve were conducted to further probe the involvement of the CXCR2 mechanism in neurophysiological attributes of chemotherapy induced peripheral neuropathy experienced by humans.

Male Sprague-Dawley rats (109-167 g, ˜5weeks old) were housed in cages in groups of three in a controlled environment of constant temperature and moisture (temperature: 25±3° C., light: dark cycle of 12:12 hours) with food (25 g/rat/day of LabDiet 5053) and water available ad libitum.

Compound Preparation: Compound of Formula (A) was prepared as a 2 mg/mL suspension in 1% methylcellulose (MC) (w/v). MC (Sigma Cat #423238) was made up in water at 1% (w/v) (1 g per 100 mL water) and left on a stirrer until completely dissolved. The formulation was prepared every 6-8 days in 400-700 mL each time and stored at 4° C. protected from light. The dosing volume used was 10 mL/kg to give a 20 mg/kg p.o. dose. Vehicle treated animals were dosed b.i.d. with 1% MC w/v at 10 mL/kg p.o.

Oxaliplatin (Selleckchem) was added to 5% glucose (Sigma Aldrich) solution and sonicated for 30 mins until dissolved. A fresh solution was prepared before every injection. Control vehicle animals were treated with 5% w/v Gluclose at 5ml/kg. Glucose (Sigma Aldrich Cat #G7021) was made up in milli-Q water at 5% (w/v) (5 g per 100 mL water) and left on a stirrer until completely dissolved. It was filtered using a sterile 0.2 μm filter (Thermo scientific Cat #595-4520) and stored at room temperature.

Treatment regimen with Sprague Dawley rats: Male Sprague Dawley rats were dosed daily with Compound of Formula (A) (20 mg/kg p.o. b.i.d.) or its vehicle (1% methycellulose 10 ml/kg p.o. b.i.d.) for 4 weeks (28 days) starting at Day 0. Each day, dosing was carried out once in the morning followed approximately 8 hours later by a second administration in the evening. Starting twenty-four hours after the first days pretreatment with Compound of Formula (A), animals were dosed with either oxaliplatin (4 mg/kg i.p.) or its vehicle (glucose solution 5% w/v: 1 ml/kg i.p.) under a brief period of 3% isoflurane (Abbott) anaesthesia. This treatment with oxaliplatin or its vehicle was repeated twice per week for 4 weeks (on Days 1, 3, 8, 10, 15,17, 22, 24), 90 minutes after the morning pretreatment with Compound of Formula (A) or vehicle. Thus rats were randomly assigned to the following groups:

| | |
|---|---|
| Veh/Veh: | 1% MC (10 ml/kg p.o. b.i.d.)/Vehicle for oxaliplatin |
| | (Glucose solution 5% w/v; 1 ml/kg i.p.; twice per week |
| | for 4 weeks) |
| Veh/Oxa: | 1% MC (10 ml/kg p.o. b.i.d.)/oxaliplatin (4 mg/kg i.p.; |
| | twice per week for 4 weeks) |
| Compound | (A)/Oxa: Compound of Formula (A) (20 mg/kg p.o., b.i.d.)/ |
| of Formula | oxaliplatin (4 mg/kg i.p.; twice per week for 4 weeks) |

Body weight tracking: The bodyweights of rats were measured twice a week for 4 weeks on Days 1, 3, 9, 11, 16, 18, 23, and 25. The body weight values for week 5 represent the combined data for each of the groups from Days 29, 30 and 31 when the conduction velocity studies were carried out. Scientists carrying out these studies were blind to the treatment that the animals had received until all data analysis had been completed.

Data were analyzed using Graphpad Prism software. They were presented as mean values±SEM. The statistical significance of differences in parameters was determined by two-way ANOVA followed by Dunnett test. A p value of <0.05 was considered to be statistically significant.

Measurement of sciatic nerve conduction velocity: Nerve conduction velocity was measured in the right sciatic nerve between Day 29 to 31 after 4 weeks treatment with Compound of Formula (A) or its vehicle. Treatment groups were balanced across each of these days and scientists carrying out these studies were blind to the treatment that the animals had received until all data analysis had been completed. Following measurements of conduction velocity, the left sciatic nerve of the rats was collected for histology studies (see below for Methods).

Rats were anesthetized with chloral hydrate (350 mg/kg, 3.5 mL/kg, i.p.). When surgical depth of anesthesia had been reached, the muscle above the sciatic notch and ankle of the right hind limb was carefully exposed. The right sciatic nerve was carefully identified and conduction velocity was measured by recording action potentials from an electrode placed between the second and third digits as described by Jamieson et al., Br. J. Cancer, 88(12):1942-7 (2003), and electrically stimulating the nerve (5V, 0.5 s, single-wave pulses) via a platinum wire electrode at the sciatic notch and ankle. The length between the sciatic notch and ankle was measured.

The action potentials were recorded using a CED Micro1401-3 scientific digital data recorder with CED 1902 mk IV programmable amplifier/filter and a DS2A-MK.II Isolated Stimulator-Constant Voltage (Cambridge Electronic Design Limited), data was analyzed by CED Signal for Windows software-version 6 (Cambridge Electronic Design Limited).

The nerve conduction velocity was calculated as the length between sciatic notch and ankle divided by the difference between the time latencies at the stimulation sites [latency (sciatic notch)−latency (ankle)].

Data were analyzed using Graphpad Prism software. They were presented as mean values±SEM. The statistical significance of differences in parameters was determined by one-way ANOVA followed by Tukey test. A p value of <0.05 was considered to be statistically significant.

Histological assessment of the Sciatic Nerve: Following measurements of conduction velocity, the left sciatic nerves of the rats were collected for histology studies. Scientists carrying out these studies were blind to the treatment that the animals had received until all data analysis had been completed.

A 1 cm segment of the left sciatic nerve (distal end) was located and carefully dissected out. The nerve was fixed in 4% paraformaldehyde (Sigma Aldrich Cat no: P6148) overnight followed by 0.2% glycine (Sigma Aldrich Cat no: 15527) overnight. After washing with Phosphate Buffered Saline [Sodium phosphate monobasic (Sigma Aldrich Cat no: 04270); Sodium phosphate dibasic (Sigma Aldrich Cat no: 30427); Sodium Chloride (BDH Cat no: 7647-14-5)], the sciatic nerve was post-fixed in 2% osmium tetraoxide for 2 hours. Following dehydration from 30% to 70% ethanol, the nerve sample was embedded in paraffin wax and the nerve was cut into 2 μm thickness sections (using a Leica microtome), mounted on a slide (Superior Maerienfeld) and air-dried at 58° C. The sections were then immersed in 100% xylene (BDH; CAS No 1330-20-7) followed by re-hydration from 100% to 70% ethanol (Merck; CAS no: 64-17-5) and then 100% Milli-Q water. The nerve was then stained in 1% toluidine blue and after washing with water and 75% ethanol, the slide was mounted with a cover slip. The first clear and intact nerve section was chosen for analysis. The image was captured using an automated upright compound microscope (Leica DM 6000B). The cross sectional area of the inner and entire area of nerve fibers of the sciatic nerves (including the myelin sheath) was measured automatically using a IN Cell Analyzer 6000 (GE Healthcare) as described by Di Cesare Mannelli et al., J. Pain, 14(12): 1585-600 (2013). The ratio of inner area over entire area of nerve fibers of sciatic nerves was then calculated as an indicator of thickness of myelin sheath of nerves.

Data were analyzed using Graphpad Prism software. They were presented as mean values±SEM. The statistical significance of differences in parameters was determined by one-way ANOVA followed by Tukey test. A p value of <0.05 was considered to be statistically significant.

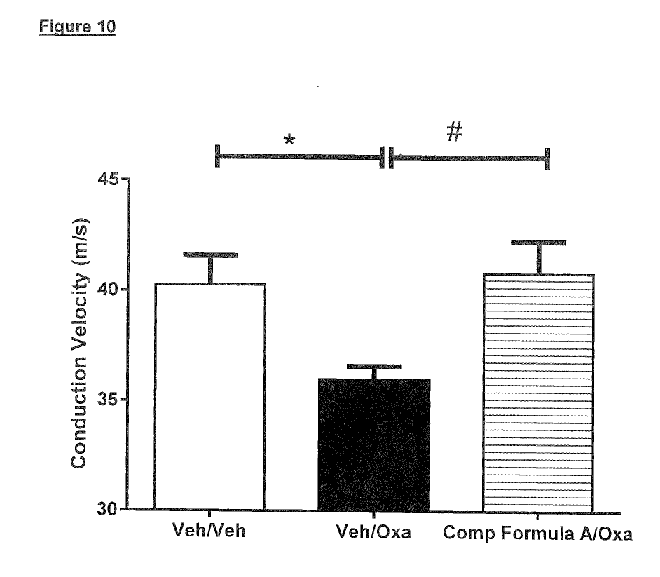

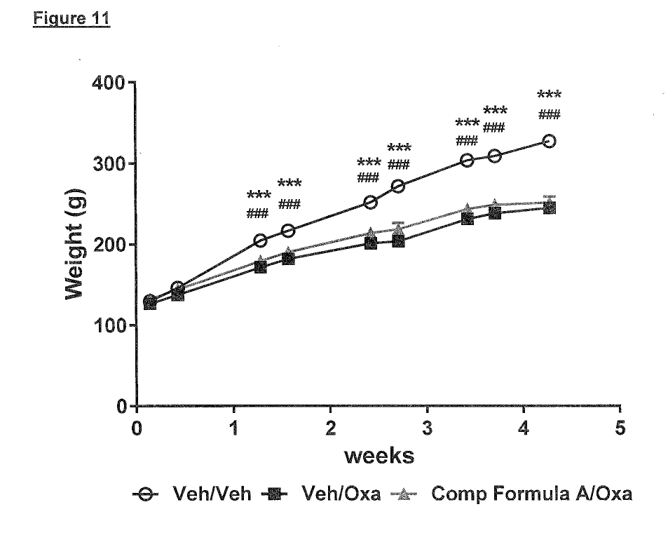

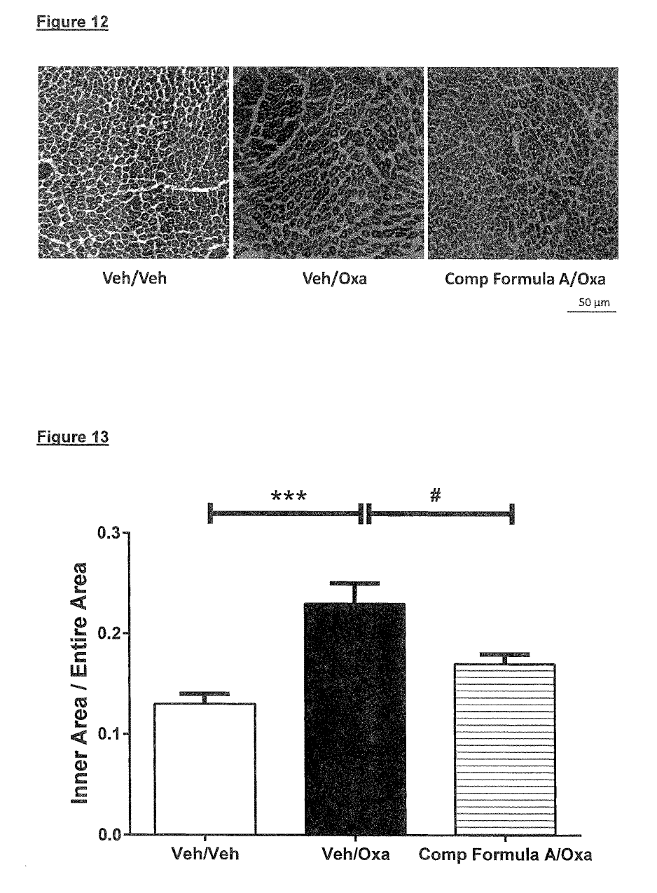

Results: With the chronic rat CIPN model, the body weight, the conduction velocity and the thickness of myelin sheath of the sciatic nerve were examined. Nerve conduction velocity was measured on Day 29, Day 30 and Day 31. No difference was seen in the conduction velocities in each of the treatment groups between Days 29, 30 or 31. Oxaliplatin-treated rats exhibited a significantly lower conduction velocity between sciatic notch and ankle (35.99±0.59 m/s) compared to vehicle-treated control (40.28±1.30 m/s) (p<0.05). Compound of Formula (A) treatment significantly prevented the reduction in conduction velocity induced by oxaliplatin in rats (40.83±1.45 m/s) (p<0.05) (FIG. 10). No significant difference was observed in body weight between the Veh/Oxa-treated and Compound of Formula (A)/Oxa-treated groups (FIG. 11). The effect of Compound of Formula (A) on the thickness of the myelin sheath of sciatic nerves was studied using histological staining. Representative cross-sections of the nerve fibers from all groups are shown in FIG. 12. The thickness of the myelin sheath of the nerves was quantified. From FIG. 13, oxaliplatin-treated rats showed a significant thinning of the myelin sheath as indicated by a higher ratio of inner area over entire area of the nerve fibers (0.23±0.02) compared to vehicle control (0.13±0.01) (p<0.001). Oral administration of Compound of Formula (A) to oxaliplatin-treated rats exhibited an increased thickness of myelin sheath of sciatic nerves when compared to Veh/Oxa-treated as indicated by lower ratio of inner area over entire area of the nerve fibers (0.17±0.01) (p<0.05). Thus the prophylactic treatment with Compound of Formula (A) inhibited the sciatic nerve conduction velocity impairment induced by the oxaliplatin treatment. FIG. 10: Values are mean±SEM. *P<0.05 indicates significance of the Veh/Oxa compared to the Veh/Veh group, #p<0.05 indicates significance of the Compound of Formula (A)/Oxa compared to the Veh/Oxa group, One-way ANOVA. Veh/Veh, n=13; Veh/Oxa, n=12; Compound of Formula (A)/Oxa, n=12. It is shown in FIG. 11 that Compound of Formula (A) did not affect oxaliplatin-induced body weight changes. A significant difference was observed in body weight between vehicle/vehicle and vehicle/oxaliplatin-treated groups from the 2 week time point. The weights were measured twice a week for 4 weeks. The body weight values for week 5 represent the combined data for each of the groups from Days 29, 30 and 31, when the conduction velocity studies were carried out. No significant difference was found between vehicle/oxaliplatin and compound of Formula (A)/oxaliplatin-treated groups at any of the time points studied. Values are mean±SEM. ***p<0.001 indicates significance of Veh/Veh compared to Veh/Oxa, ###p<0.001 indicates significance of Veh/Veh compared with Compound D/Oxa treated rats; two-way ANOVA followed by Dunnett test, n=13 (Veh/Veh), n=12 (Veh/Oxa), n=12 (Compound D/Oxa). FIGS. 12 and 13 show that Compound of Formula (A) prevented the reduction of thickness of myelin of sciatic nerve by oxaliplatin. FIG. 12 showing images from each group taken using a light microscope at 40× magnification. FIG. 13 graphically shows the thickness of myelin sheath of the nerve fibers. The ratio of the cross sectional area of the inner nerve fibre to the total cross sectional area of the entire nerve fibre the sciatic nerve (including the myelin sheath) was assessed. Oxaliplatin reduced the thickness of myelin sheath of sciatic nerves in the rats, an effect that was reversed by treatment with Compound of Formula (A). Values are mean±SEM. ***p<0.001 indicates significance of Veh/Oxa compared to Vehicle/Vehicle group, #p<0.05 indicates significance of Compound D/Oxa compared to Veh/Oxa treated rats; One-way ANOVA followed by Tukey test, n=12 (Veh/Veh), n=12 (Veh/Oxa), n=12 (Compound of Formula (A)/Oxa).

Summary: Oxaliplatin induced a peripheral neuropathy in rats as assessed by the reductions in the nerve conduction velocity and the thickness of the nerve myelin sheath. Pre-treatment with Compound of Formula (A) prevented the reduction of sciatic nerve conduction velocity in oxaliplatin-treated rats but did not affect the decrease in body weight induced by oxaliplatin treatment. Compound of Formula (A) also ameliorated the changes in the myelin sheath of the sciatic nerves induced by oxaliplatin.

The results of these studies show that Compound of Formula (A) prevents oxaliplatin-induced changes in behavioural features of hypersensitivity to mechanical stimuli, myelin status of the sciatic nerve and slowing of nerve conduction velocity in rats. These are characteristics of chemotherapy induced peripheral neuropathy in patients.

k) Proof of Mechanism Clinical Study

It is intended to conduct a proof of mechanism clinical study to further demonstrate the effect of the Compound of Formula (A) in treating CIPN in human patients. A possible study design is as follows:

The clinical study design will be: double blind, placebo controlled parallel group proof of mechanism study in patients suffering from colorectal cancer scheduled to start oxaliplatin-containing chemotherapy treatment.

The outline of the project will likely include that subjects will attend the clinical centre for the anticancer chemotherapy every two weeks. Routine laboratory examination will be performed before each oxaliplatin dose in compliance with the clinical centre procedures. The subjects will be instructed to start taking Compound of Formula (A) two days before the oxaliplatin iv infusion. During the first six oxaliplatin infusions the eligible subjects will be instructed to consume a dose of Compound of Formula (A) (x tablets×mg or placebo) once a day for 7 days in the morning. The dosing regimen of Compound of Formula (A) is designed to achieve the CXCR2 antagonist maximum effect during the systemic peak of oxaliplatin. In this first study the intermittent dosing will maximize the therapeutic efficacy and minimize the potential of any side effects through prolonged co-administration with oxaliplatin. After the end of the sixth cycle the subjects will stop the treatment with Compound of Formula (A) or placebo and will continuing the chemotherapy regimen where clinically appropriate.

During the first 7 chemotherapy cycles, while at the clinical centre, assessments will include safety, tolerability and pharmacokinetics. Clinical assessments and primary electrophysiological measures on nerve excitability profiles will be made at baseline and between 24-48 hrs after the oxaliplatin infusion in cycles 1, 3, 5, 6 and 7. All visits to the clinical centre will include clinical assessments using the NCI-CTCv4 and Total Neuropathy Score and subset [TNSc; Cavaletti G F. B., J. Peripher. Nerv. Syst., September, 12(3), 210-5 (2007); Cornblath D R, Neurology, 53(8), 1660 (1999)]. Oncology measures will be monitored as per standard of care.

Primary measures will include:
Changes of sensory excitability (superexcitability %) measured as difference between pre-dose of chemotherapy cycle 1 and prior to the chemotherapy cycle that follows the last Compound of Formula (A)/placebo dose. Safety of Compound of Formula (A): monitoring of adverse events, clinical signs, safety laboratory and vital signs.
PK of Compound of Formula (A).

### Compound of Formula (D): 1-(4-Chloro-3-((1,4-dimethylpiperidin-4-yl)sulfonyl)-2-hydroxphenyl)-3-(2-chloro-3-fluorophenyl)urea, trifluoroacetic acid salt

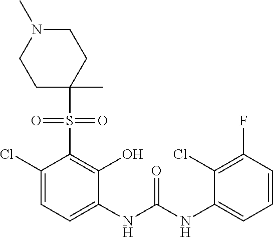 [SMILES: CN1CCC(C)(S(=O)(=O)c2c(Cl)ccc(NC(=O)Nc3cccc(F)c3Cl)c2O)CC1]

To a solution of 6-amino-3-chloro-2-[(1,4-dimethyl-4-piperidinyl)sulfonyl]phenol, dihydrochloride (Intermediate D1) (0.30 g) in 1,4-dioxane (10 mL) and water (1 mL) was added NaHCO₃ (0.193 g). The mixture was stirred at RT for 10 min. Then 2-chloro-1-fluoro-3-isocyanatobenzene (0.131 g) was added. Stirring was continued for additional 10 min. Afterwards, the reaction mixture was diluted with water (50 mL), extracted with EA (2×50 mL). The solution was then washed with brine, dried over anhydrous Na₂SO₄ and purified by MDAP to give the title product (130 mg).

### Intermediate D1: 6-amino-3-chloro-2-((1,4-dimethylpiperidin-4-yl)sulfonyl)phenol, dihydrochloride

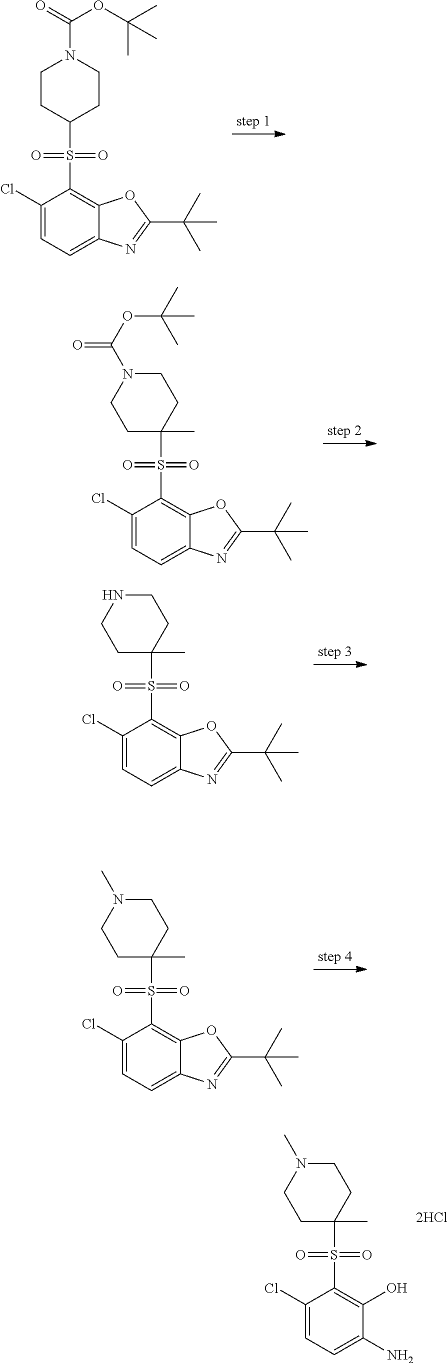

Step 1: To a solution of tert-butyl 4-((2-(tert-butyl)-6-chlorobenzo[d]oxazol-7-yl)sulfonyl)piperidine-1-carboxylate (0.35 g) (Intermediate D2) in tetrahydrofuran (THF) (25 mL) at −78° C. was added n-butyllithium (0.383 mL, 2.0 M in cyclohexane). The mixture was stirred at −78° C. for 1 h. Then MeI (0.048 mL) was added. Stirring was continued for 4 h at −78° C. Afterwards, the reaction was quenched with aq. NH₄Cl, extracted with EA (2×50 mL). The solution was then washed with brine, dried over anhydrous Na₂SO₄, and concentrated to give tert-butyl 4-((2-(tert-butyl)-6-chlorobenzo[d]oxazol-7-yl)sulfonyl)-4-methylpiperidine-1-carboxylate (0.4 g).

Step 2: To a solution of tert-butyl 4-((2-(tert-butyl)-6-chlorobenzo[d]oxazol-7-yl)sulfonyl)-4-methylpiperidine-1-carboxylate (0.35 g) in dichloromethane (DCM) (20 mL) was added TFA (0.572 mL). The mixture was stirred at RT overnight. The resulting solution was concentrated in vacuo to give 2-(tert-butyl)-6-chloro-7-((4-methylpiperidin-4-yl)sulfonyl)benzo[d]oxazole, trifluoroacetic acid salt (0.35 g).

Step 3: To a solution of 2-(tert-butyl)-6-chloro-7-((4-methylpiperidin-4-yl)sulfonyl)benzo[d]oxazole, trifluoroacetic acid salt (0.35 g) in N,N-dimethylformamide (DMF) (10 mL) was added AcOH (0.054 mL) and formaldehyde (0.788 mL). Then the reaction mixture was cooled to 0° C. and stirred for 10 min. Sodium triacetoxyborohydride (0.6 g) was then added portionwise. After completion of the reaction, the mixture was quenched with sat. aq. NaHCO₃ (20 mL) and extracted with EA (2×50 mL). The solution was then washed with brine, dried over anhydrous Na₂SO₄, and concentrated to give 2-(tert-butyl)-6-chloro-7-((1,4-dimethylpiperidin-4-yl)sulfonyl)benzo[d]oxazole (0.3 g).

Step 4: To a solution of 2-(tert-butyl)-6-chloro-7-((1,4-dimethylpiperidin-4-yl)sulfonyl)benzo[d]oxazole (0.3 g) in 1,4-dioxane (10 mL) and water (10 mL) was added HCl (0.640 mL, 37% in water). The mixture was heated at 120° C. for 3 hours. Afterwards, the resulting solution was concentrated in vacuo to give the title product (0.3 g), which was used in the next step without further purification.

### Intermediate D2: tert-butyl 4-((2-(tert-butyl)-6-chlorobenzo[d]oxazol-7-yl)sulfonyl)piperidine-1-carboxylate

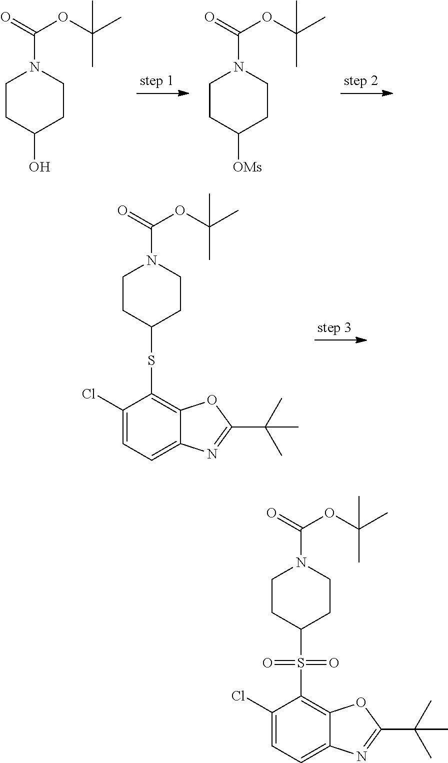

Step 1: To a solution of tert-butyl 4-hydroxypiperidine-1-carboxylate (10.0 g) in DCM (100 mL) was added TEA (13.9 mL), followed by addition of MsCl (4.7 mL) in an ice bath. The mixture was stirred at RT for 4 hours. Water (100 mL) was added. The organic layer was separated, dried over sodium sulfate, filtered and concentrated to afford tert-butyl 4-((methylsulfonyl)oxy)piperidine-1-carboxylate (13.9 g) as a white solid. MS(ES⁺) m/z 302 (MNa⁺).

Step 2: To a solution of sodium 2-(tert-butyl)-6-chlorobenzo[d]oxazole-7-thiolate (12.0 g) and tert-butyl 4-((methylsulfonyl)oxy)piperidine-1-carboxylate (13.9 g) in DMF (100 mL) was added potassium carbonate (6.3 g). The mixture was stirred at 80° C. for 2 hours. EA (200 mL) was added. The organic phase was washed with brine (4×200 mL). The combined organic layers were dried over sodium sulfate, filtered and concentrated to afford tert-butyl 4-((2-(tert-butyl)-6-chlorobenzo[d]oxazol-7-yl)thio)piperidine-1-carboxylate (19.3 g) as a yellow oil. MS(ES⁺) m/z 369 (M−t−Bu+H+H⁺).

Step 3: To a solution of tert-butyl 4-((2-(tert-butyl)-6-chlorobenzo[d]oxazol-7-yl)thio)piperidine-1-carboxylate (19.3 g) in DCM (50 mL) was added mCPBA (20.4 g) at 0° C. After stirring at RT overnight, the mixture was quenched with aq. NaHCO₃ solution and aq. Na₂S₂O₃ solution, and then extracted with EA (2×150 mL). The combined organic layers were washed, dried and concentrated. The crude was purified by recrystallization in methanol/H₂O to afford tert-butyl 4-((2-(tert-butyl)-6-chlorobenzo[d]oxazol-7-yl)sulfonyl)piperidine-1-carboxylate (14.0 g). MS(ES⁺) m/z 479 (MNa⁺).

### Compound of Formula (E): (R)-1-(4-chloro-2-hydroxy-3-((4-methyltetrahydro-2H-pyran-4-yl)sulfonyl)phenyl)-3-(2-chlorocyclopent-2-en-1-yl)urea

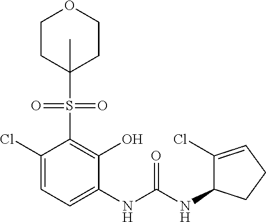

To a solution of 6-amino-3-chloro-2-((4-methyltetrahydro-2H-pyran-4-yl)sulfonyl)phenol, hydrochloride (50 mg) (Intermediate E1) in pyridine (5 mL) was added fresh (R)-1-chloro-5-isocyanatocyclopent-1-ene (Intermediate E2, 0.03 M in toluene, 5 mL) and the resulting mixture was stirred at RT overnight. The mixture was quenched with ethanol (5 mL) and concentrated under reduced pressure. The residue was purified with MDAP (basic condition) to afford the title compound (34 mg); ¹H-NMR (400 MHz, DMSO-d₆) δ ppm 10.36 (br. s., 1H), 8.39 (d, J=8.8 Hz, 1H), 8.20 (s, 1H), 7.35 (d, J=8.8 Hz, 1H), 7.13 (d, J=9.0 Hz, 1H), 5.96-6.02 (m, 1H), 4.69-4.80 (m, 1H), 3.84 (dd, J=11.7, 4.4 Hz, 2H), 3.49 (t, J=11.2 Hz, 2H), 2.22-2.46 (m, 3H), 2.04 (td, J=12.5, 5.1 Hz, 2H), 1.61-1.74 (m, 1H), 1.58-1.61 (m, 1H), 1.54-1.58 (m, 1H), 1.50 (s, 3H); MS(ES⁺) m/z 449 (MH⁺).

### Intermediate E1: 6-amino-3-chloro-2-((4-methyltetrahydro-2H-pyran-4-yl)sulfonyl)phenol, hydrochloride

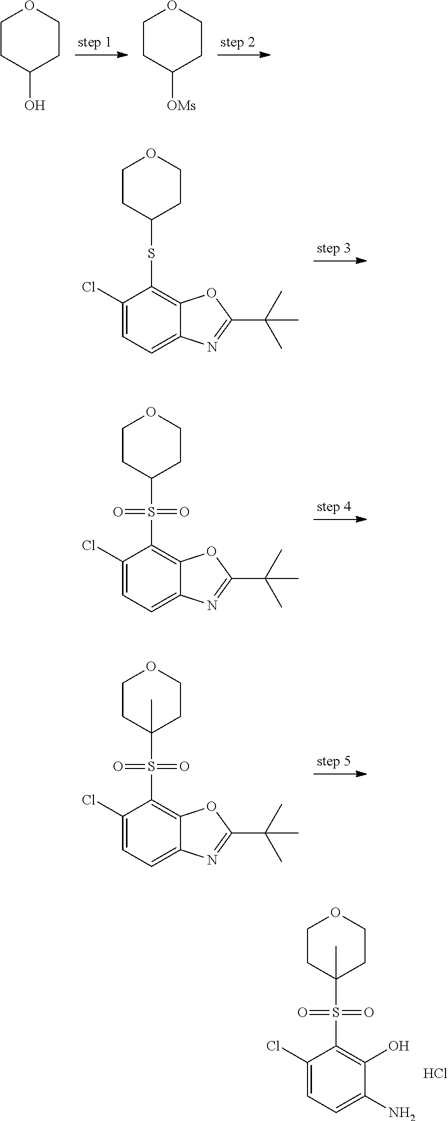

Step 1: To a solution of tetrahydro-2H-pyran-4-ol (10.0 g) in DCM (200 mL) was added TEA (12.9 g) and methanesulfonyl chloride (11.3 g). The mixture was stirred at 0° C. for 1 hour, and then washed with H₂O. The organic layer was dried over Na₂SO₄ and concentrated to afford tetrahydro-2H-pyran-4-yl methanesulfonate (15.5 g).

Step 2: To a solution of 2-(tert-butyl)-6-chlorobenzo[d]oxazole-7-thiol (18.0 g) and Cs₂CO₃ (12.1 g) in acetonitrile (5 mL) was added tetrahydro-2H-pyran-4-yl methanesulfonate (14.8 g). The mixture was stirred at 90° C. for 16 hours. After cooling to RT, the mixture was concentrated. The residue was partioned between EA (100 mL) and H₂O (100 mL). The organic layer was dried and concentrated to afford 2-(tert-butyl)-6-chloro-7-((tetrahydro-2H-pyran-4-yl)thio)benzo[d]oxazole (24.0 g). MS(ES⁺) m/z 326 (MH⁺).

Step 3: To a solution of 2-(tert-butyl)-6-chloro-7-((tetrahydro-2H-pyran-4-yl)thio)benzo[d]oxazole (24.0 g) in DCM (1000 mL) was added mCPBA (31.8 g). The mixture was stirred at 15° C. for 2 hours, and then quenched with aq. Na₂SO₃ solution. The pH was adjusted to ˜7. The organic layer was dried and concentrated to afford 2-(tert-butyl)-6-chloro-7-((tetrahydro-2H-pyran-4-yl)sulfonyl)benzo[d]oxazole (18.0 g).

Step 4: To a solution of 2-(tert-butyl)-6-chloro-7-((tetrahydro-2H-pyran-4-yl)sulfonyl)benzo[d]oxazole (5.0 g) in THF (50 mL) was added BuLi (2.5 M in hexanes, 6.2 mL) at −78° C. under a nitrogen atmosphere. The mixture was stirred at −78° C. for 45 min. MeI (2.2 g) was added. The reaction mixture was stirred at −78° C. for 1 hour, and then quenched with aq. NH₄Cl solution. The organic layer was dried and concentrated. The residue was purified by column chromatography to afford 2-(tert-butyl)-6-chloro-7-((4-methyltetrahydro-2H-pyran-4-yl)sulfonyl)benzo[d]oxazole (4.8 g). MS(ES⁺) m/z 372 (MH⁺).

Step 5: To a solution of 2-(tert-butyl)-6-chloro-7-((4-methyltetrahydro-2H-pyran-4-yl)sulfonyl)benzo[d]oxazole (1.0 g) in 1,4-dioxane (10 mL) was added aq. HCl solution (37%, 10 mL). After refluxed at 110° C. for 4 hours, the mixture was concentrated to afford the title compound (1.0 g) as a gray solid. MS(ES⁺) m/z 306 (MH⁺).

### Intermediate E2: (R)-1-chloro-5-isocyanatocyclopent-1-ene

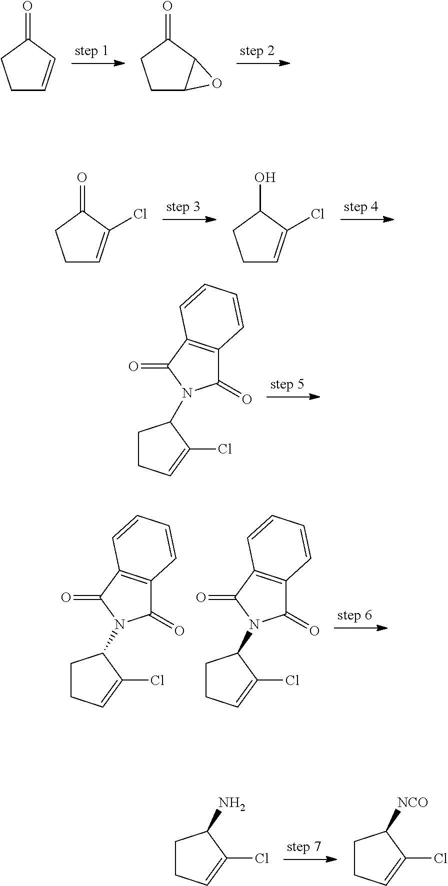

Step 1: To a solution of cyclopent-2-enone (1.2 g) in methanol (10 mL) was added hydrogen peroxide solution (30%, 0.5 g). The resulting mixture was stirred at RT overnight. Cold water (30 mL) was added and the resulting mixture was neutralized with sat. NaHCO₃ solution. The aqueous layer was extracted with DCM (2×100 mL). The combined organic layers were dried over Na₂SO₄, filtered and concentrated in vacuo to give 6-oxabicyclo[3.1.0]hexan-2-one (1.3 g) as a yellow oil.

Step 2: To a solution of 6-oxabicyclo[3.1.0]hexan-2-one (25 g) in methanol (10 mL) and water (3 mL) was added cerium(III) chloride heptahydrate (95 g). The resulting mixture was stirred at 70° C. for one hour. Cold water (30 mL) was added and the resulting mixture was neutralized with sat. NaHCO₃ solution. The aqueous layer was extracted with DCM (2×100 mL). The combined organic layers were dried over Na₂SO₄, filtered and concentrated in vacuo to give 2-chlorocyclopent-2-enone (29 g) as a yellow oil.

Step 3: To a solution of 2-chlorocyclopent-2-enone (600 mg) in methanol (20 mL) was added cerium(III) chloride heptahydrate (1918 mg) and NaBH₄ (195 mg). The resulting mixture was stirred at RT for one hour. Cold water (30 mL) was added and the resulting mixture was neutralized with sat. NaHCO₃ solution. The aqueous layer was extracted with DCM (2×100 mL). The combined organic layers were dried over Na₂SO₄, filtered and concentrated in vacuo to give 2-chlorocyclopent-2-enol (400 mg) as a yellow oil.

Step 4: To a solution of 2-chlorocyclopent-2-enol (20.0 g) and isoindoline-1,3-dione (37.2 g) in THF (200 mL) was added Ph₃P (66.4 g) and DIAD (49.2 mL) at 0° C. The mixture was stirred at RT overnight. The solvent was removed in vacuo and the residue was purified by column chromatography (eluting with PE:EA=10:1) to give 2-(2-chlorocyclopent-2-en-1-yl)isoindoline-1,3-dione (18.0 g) as a yellow solid. MS(ES⁺) m/z 248 (MH⁺).

Step 5: 2-(2-Chlorocyclopent-2-en-1-yl)isoindoline-1,3-dione (14 g) was purified by SFC to give (R)-2-(2-chlorocyclopent-2-en-1-yl)isoindoline-1,3-dione (5.0 g) as a white solid and (S)-2-(2-chlorocyclopent-2-en-1-yl)isoindoline-1,3-dione (5.5 g) as a yellow oil. (R)-2-(2-chlorocyclopent-2-en-1-yl)isoindoline-1,3-dione: Chiral HPLC (Column: AD-H (250*4.6 mm, 5 um); mobile phase: MeOH/CO₂=15%; Flow: 3.0 ml/min; Temperature: 40° C.): t_{R}=2.54 min, ee%=100%; ¹H-NMR (400 MHz, CDCl₃) δ ppm 7.74-7.98 (m, 4H), 6.04 (d, J=2.2 Hz, 1H), 5.32 (dd, J=9.3, 1.9 Hz, 1H), 2.77 (ddd, J=9.4, 8.0, 3.0 Hz, 1H), 2.41-2.62 (m, 2H), 2.22-2.39 (m, 1H); (S)-2-(2-chlorocyclopent-2-en-1-yl)isoindoline-1,3-dione: Chiral HPLC (Column: AD-H (250*4.6 mm, 5 um); mobile phase: MeOH/CO₂=15%; flow: 3.0 ml/min; temperature: 40° C.): t_{R}=3.04 min, ee%=100%; ¹H-NMR (400 MHz, CDCl₃) δ ppm 7.32-8.36 (m, 4H), 6.03 (d, J=2.2 Hz, 1H), 5.31 (dd, J=9.3, 1.8 Hz, 1H), 2.77 (ddd, J=9.4, 8.0, 3.0 Hz, 1H), 2.40-2.64 (m, 2H), 2.32 (ddd, J=14.2, 8.7, 3.8 Hz, 1H).

Step 6: To a solution of (R)-2-(2-chlorocyclopent-2-en-1-yl)isoindoline-1,3-dione (3.5 g) in ethanol (100 mL) was added hydrazine.H₂O (0.7 mL). After refluxing for 3 hours, the reaction mixture was cooled to RT. The precipitate was filtered and rinsed with EtOH (10 mL). The filtrate was concentrated to remove half of solvent. To the solution was added HCl in ether (1 M, 20 mL) and concentrated to afford (R)-2-chlorocyclopent-2-enamine as a hydrochloride salt (2.0 g). ¹H-NMR (400 MHz, DMSO-d₆) δ ppm 8.53 (s, 3H), 6.22 (s, 1H), 4.19 (d, J=5.8 Hz, 1H), 2.49-2.54 (m, 1H), 2.26-2.45 (m, 2H), 1.90-2.04 (m, 1H).

Step 7: To a solution of (R)-2-chlorocyclopent-2-enamine hydrochloride salt (600 mg) in toluene (15 mL) was added bis(trichloromethyl) carbonate (694 mg). The mixture was stirred at 120° C. for 4 hours. The mixture was then cooled to RT to afford a toluene solution of (R)-1-chloro-5-isocyanatocyclopent-1-ene. This solution should be synthesized freshly every time.

### Compound of Formula (F): (R)-1-(3-(tert-butylsulfonyl)-4-cyano-2-hydroxyphenyl)-3-(2-methylcyclopent-2-en-1-yl)urea

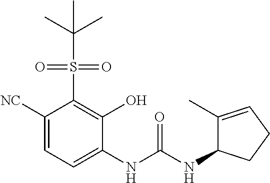 [SMILES: CC1=CCC[C@H]1NC(=O)Nc1ccc(C#N)c(S(=O)(=O)C(C)(C)C)c1O]

To a solution of 4-amino-2-(tert-butylsulfonyl)-3-hydroxybenzonitrile (Intermediate F1, 450 mg) in pyridine (20 mL) was added fresh (R)-5-isocyanato-1-methylcyclopent-1-ene (Intermediate F2, 327 mg) in toluene (20 mL). The reaction mixture was stirred at RT overnight. The mixture was quenched with water (5 mL) and concentrated. The residue was purified with MDAP (acidic condition) to afford (R)-1-(3-(tert-butylsulfonyl)-4-cyano-2-hydroxphenyl)-3-(2-methylcyclopent-2-en-1-yl)urea (120 mg). ¹H-NMR (400 MHz, DMSO-d₆) δ ppm 10.13 (br. s., 1H), 8.51-8.64 (m, 2H), 7.60 (d, J=8.6 Hz, 1H), 7.34 (d, J=8.3 Hz, 1H), 5.52 (s, 1H), 4.54 (d, J=6.8 Hz, 1H), 2.11-2.37 (m, 3H), 1.66 (s, 3H), 1.46-1.60 (m, 1H), 1.30-1.46 (m, 9H); MS(ES⁺) m/z 378 (MH⁺).

### Intermediate F1: 4-amino-2-(tert-butylsulfonyl)-3-hydroxybenzonitrile

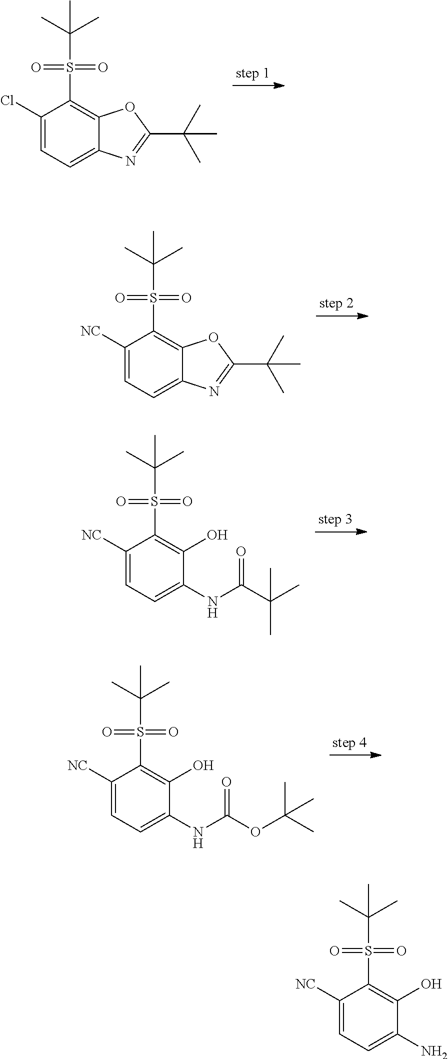

Step 1: The reaction was carried out in five batches (600 mg each) for microwave synthesis, and then combined for purification: A mixture of 2-(tert-butyl)-7-(tert-butylsulfonyl)-6-chlorobenzo[d]oxazole (Intermediate F3, 0.6 g) and copper(I) cyanide (1.6 g) in NMP (4 mL) was stirred at 180° C. in the microwave for 90 min. After cooling, the five batches were combined, and diluted with EA (100 mL) and water (100 mL). After filtration, the organic layer was separated, washed, dried, filtered and concentrated. The residue was purified by column chromatography (eluting with PE:EA=1:0 to 7:3) to afford 2-(tert-butyl)-7-(tertbutylsulfonyl)benzo[d]oxazole-6-carbonitrile (1.0 g). MS(ES⁺) m/z 321 (MH⁺).

Step 2: To a solution of 2-(tert-butyl)-7-(tert-butylsulfonyl)benzo[d]oxazole-6-carbonitrile (1.0 g) in ethanol (25 mL) and water (25 mL) was added sodium hydroxide (0.3 g). The resulting mixture was stirred at 60° C. for 1 hour, and then concentrated under reduced pressure. The residue was diluted with water (50 mL), acidified with aq. citric acid to pH=6, and extracted with EA (2×50 mL). The combined organic layers were washed, dried, filtered and concentrated to afford N-(3-(tert-butylsulfonyl)-4-cyano-2-hydroxyphenyl)pivalamide (1.1 g). MS(ES⁺) m/z 361 (MH⁺).

Step 3: To a solution of N-(3-(tert-butylsulfonyl)-4-cyano-2-hydroxyphenyl)pivalamide (1.2 g) in THF (30 mL) was added DMAP (0.04 g) and Boc₂O (1.6 mL). The mixture was stirred at 60° C. for 2 hours. To the mixture was added hydrazine.H₂O (1.6 mL). The resulting mixture was stirred at RT overnight, diluted with water (50 mL), and extracted with EA (2×100 mL). The combined organic layers were washed, dried, filtered and concentrated. The residue was purified by column chromatography (eluting with PE:EA=1:0 to 7:3) to afford tert-butyl (3-(tert-butylsulfonyl)-4-cyano-2-hydroxyphenyl)carbamate (1.2 g). MS(ES⁺) m/z 355 (MH⁺).

Step 4: To a solution of tert-butyl (3-(tert-butylsulfonyl)-4-cyano-2-hydroxyphenyl)carbamate (1.2 g) in DCM (25 mL) was added TFA (2.6 mL). The resulting mixture was stirred at RT overnight. DCM was removed. The residue was basified with aq. NaHCO₃ solution (pH=8), and extracted with EA (2×50 mL). The combined organic layers were washed, dried, filtered and concentrated to afford the title compound (0.8 g). MS(ES⁺) m/z 255 (MH⁺).

### Intermediate F2: (R)-5-isocyanato-1-methylcyclopent-1-ene

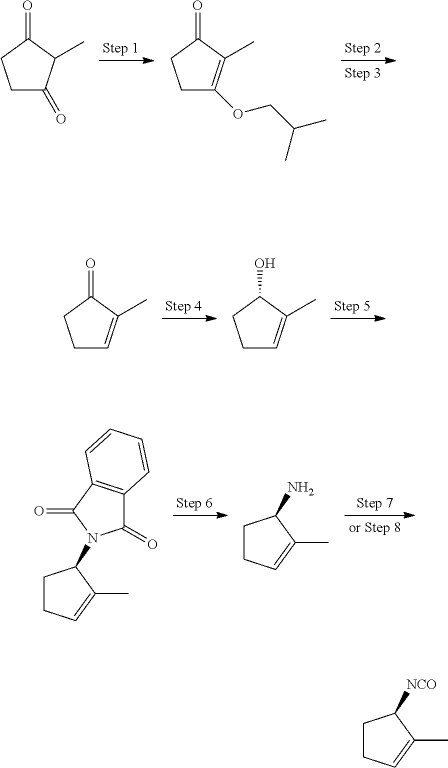

Step 1: A solution of 2-methylcyclopentane-1,3-dione (27.0 g), 2-methylpropan-1-ol (62.5 g) and TsOH (4.6 g) in benzene (500 mL) was heated to reflux overnight. The solvent was removed in vacuo and the residue was distilled under vacuum to give 3-isobutoxy-2-methylcyclopent-2-enone (34.5 g) as a yellow oil. ¹H-NMR (400 MHz, CDCl₃) δ ppm 3.92 (d, J=6.6 Hz, 2H), 2.53-2.70 (m, 2H), 2.32-2.52 (m, 2H), 2.04 (dp, J=13.3, 6.7 Hz, 1H), 1.64 (t, J=1.5 Hz, 3H), 1.01 (d, J=6.7 Hz, 6H); MS(ES⁺) m/z 169 (MH⁺).

Step 2: To a solution of 3-isobutoxy-2-methylcyclopent-2-enone (34.5 g) in DCM (300 mL) was added DIBAL-H (1 M in hexane, 250 mL) dropwise at 0° C. The reaction mixture was stirred at this temperature for 90 min. The reaction was quenched with water and then partitioned between DCM (200 mL) and HCl solution (1 M, 100 mL). The aqueous layer was extracted with DCM (2×200 mL). The combined organic layers were washed with sat. sodium bicarbonate solution (100 mL), brine (200 mL), dried over Na₂SO₄, filtered and concentrated in vacuo to give a mixture of 2-methylcyclopent-2-enol and 2-methylcyclopent-2-enone (27.0 g) as a yellow oil.

Step 3: A mixture of 2-methylcyclopent-2-enol (27.0 g) and manganese (IV) oxide (5.0 g) in diethyl ether (200 mL) was stirred at RT overnight. The mixture was filtered and the filtrate was concentrated in vacuo. The residue was distilled in vacuo to give 2-methylcyclopent-2-enone (16.5 g) as a colorless oil.

Step 4: To a solution of (R)-1-methyl-3,3-diphenylhexahydropyrrolo[1,2-c][1,3,2]oxazaborole (1 M in toluene, 31.8 mL) in absolute THF (20 mL) was added carefully 2-methylcyclopent-2-enone (15.3 g) and BH₃ (1 M in THF, 111 mL). The mixture was stirred for one hour. Methanol (150 ml) was added and the resulting mixture was diluted with brine. The aqueous layer was extracted with DCM (2×200 mL). The combined organic layers were dried over Na₂SO₄, filtered and concentrated in vacuo to give (S)-2-methylcyclopent-2-enol (unknown ee%, 16.0 g) as a yellow oil.

Step 5: To a solution of (S)-2-methylcyclopent-2-enol (16.0 g) and isoindoline-1,3-dione (36.0 g) in THF (240 mL) was added triphenylphosphine (77.0 g) under N₂. The mixture was cooled to 0° C. Diisopropyl azodicarboxylate (63.4 mL) was added dropwise to the mixture. After stirring for 30 min, the mixture was stirred at 0° C. overnight. The solvent was removed and the residue was purified by column chromatography (eluting with PE:EA=10:1) to give a crude product, which was purified by SFC to afford (R)-2-(2-methylcyclopent-2-en-1-yl)isoindoline-1,3-dione (10.6 g, >98% ee) as a white solid. Chiral HPLC (Column: AD-H, 4.6*250 mm, 5 μm, MeOH/CO₂=10%, column temperature: 40° C., CO₂ flow rate: 2.7 mL/min): t_{R}=2.17 min, ee%: >98%; ¹H-NMR (400 MHz, CDCl₃) δ ppm 7.83 (dd, J=5.4, 3.1 Hz, 2H), 7.76-7.61 (m, 2H), 5.68 (s, 1H), 5.21 (d, J=7.4 Hz, 1H), 2.69 (dd, J=5.7, 3.5 Hz, 1H), 2.42-2.30 (m, 2H), 2.22-2.12 (m, 1H), 1.61 (s, 3H); MS (ES⁺) m/z 228 (MH⁺).

Step 6: To a solution of (R)-2-(2-methylcyclopent-2-en-1-yl)isoindoline-1,3-dione (10.7 g) in ethanol (150 mL) was added hydrazine.H₂O (3.0 mL). After refluxing for 3 hours, the reaction mixture was cooled to RT. The precipitate was filtered and the filter cake was rinsed with EtOH (10 mL). To the filtrate was added HCl in dioxane (4 M, 5 mL) and the mixture was concentrated. The resulting residue was dissolved in water, then freeze dried to afford (R)-2-methylcyclopent-2-enamine as the hydrochloride salt (6.3 g) as a brown solid, which was used in the next step without purification.

Step 7: To a solution of (R)-2-methylcyclopent-2-enamine hydrochloride salt (420 mg) in toluene (30 mL) was added triphosgene (560 mg). The resulting mixture was stirred at 110° C. for 6 hours. The mixture was then cooled to RT to afford a toluene solution of (R)-5-isocyanato-1-methylcyclopent-1-ene. This solution should be synthesized freshly every time.

Step 8: To a solution of (R)-2-methylcyclopent-2-enamine hydrochloride salt (23 mg) in DCM (3 mL) and sat. NaHCO₃ aqueous solution (3 mL) was added bis(trichloromethyl) carbonate (18 mg) at 0° C. The mixture was stirred for 2 hours at 0° C. to 25° C. The resulting two layers were separated, and the aqueous layer extracted with DCM (60 mL). The combined organic layers were washed with brine (15 mL), dried over Na₂SO₄, filtered and concentrated under reduced pressure to give (R)-5-isocyanato-1-methylcyclopent-1-ene (20 mg) as a white solid, which was used directly in the next step without further purification. MS(ES⁺) m/z 141 (MH⁺) (M: the urea derivative with ammonium hydroxide).

### Intermediate F3: 2-(tert-butyl)-7-(tert-butylsulfonyl)-6-chlorobenzo[d]oxazole

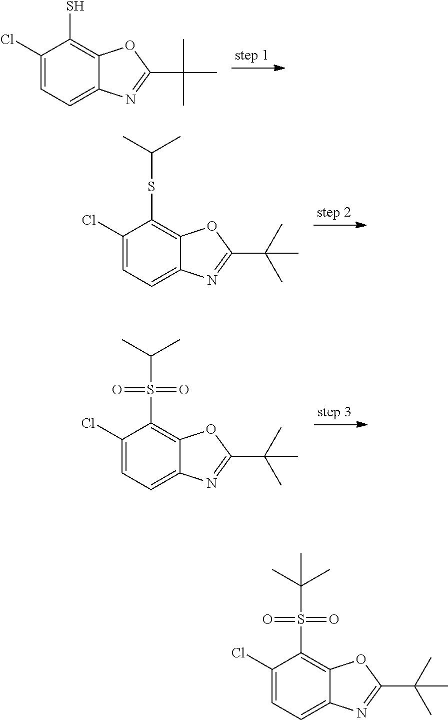

Step 1: To a solution of 2-(tert-butyl)-6-chlorobenzo[d]oxazole-7-thiol (3.0 g) in DMF (30 mL) was added 2-iodopropane (2.1 g). The mixture was stirred at 100° C. for 2 hours. After cooling, the solvent was removed. The residue was purified by column chromatography to give 2-(tert-butyl)-6-chloro-7-(isopropylthio)benzo[d]oxazole (3.5 g).

Step 2: To a solution of 2-(tert-butyl)-6-chloro-7-(isopropylthio)benzo[d]oxazole (3.5 g) in DCM (40 mL) was added mCPBA (5.3 g) at 15° C. The mixture was stirred at 15° C. for 48 hours, and then quenched with sat. Na₂SO₃ solution. The organic layer was dried over Na₂SO₄, filtered and concentrated. The residue was purified by column chromatography to afford 2-(tert-butyl)-6-chloro-7-(isopropylsulfonyl)benzo[d]oxazole (3.6 g). MS(ES⁺) m/z 316 (MH⁺).

Step 3: To a solution of 2-(tert-butyl)-6-chloro-7-(isopropylsulfonyl)benzo[d]oxazole (3.0 g) in THF (10 mL) was added LiHMDS (1 M in THF, 31.7 mL). The mixture was stirred at −78° C. for 10 min, and then iodomethane (6.74 g) was added. The mixture was stirred at −78° C. for 10 min, and then quenched with aq. NH₄Cl solution and aq. HCl solution (10%). The mixture was extracted with EA. The organic layer was washed with brine, dried over Na₂SO₄ and concentrated. The crude product was purified by column chromatography to afford 2-(tert-butyl)-7-(tert-butylsulfonyl)-6-chlorobenzo[d]oxazole (F3, 2.8 g). MS(ES⁺) m/z 330 (MH⁺).

### Compound of Formula (G): 1-(4-chloro-2-hydroxy-3-((trans-3-(pyrrolidin-1-yl)cyclobutyl)sulfonyl)phenyl)-3-((R)-2-methylcyclopent-2-en-1-yl)urea, trifluoroacetic acid salt

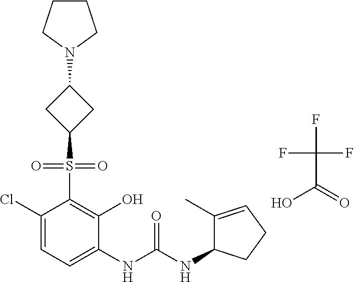

To a solution of 6-amino-3-chloro-2-((trans-3-(pyrrolidin-1-yl)cyclobutyl)sulfonyl)phenol (Intermediate G1, 80 mg) in pyridine (5 mL) was added (R)-5-isocyanato-1-methylcyclopent-1-ene (Intermediate F2, 74 mg) solution in toluene (5 mL) dropwise. The mixture was stirred at RT overnight. The mixture was concentrated and the resulting residue was redissolved in DMF (8 mL) and purified by MDAP to afford 1-(4-chloro-2-hydroxy-3-((trans-3-(pyrrolidin-1-yl)cyclobutyl)sulfonyl)phenyl)-3-((R)-2-methylcyclopent-2-en-1-yl)urea as a trifluoroacetic acid salt (21 mg) as a white solid. ¹H-NMR (400 MHz, DMSO-d₆) δ ppm 10.44 (br. s., 2H), 8.28 (br. s., 1H), 8.18 (d, J=8.8 Hz, 1H), 7.13 (d, J=8.8 Hz, 1H), 7.10 (d, J=8.6 Hz, 1H), 5.52 (s, 1H), 4.49-4.62 (m, 2H), 4.02 (quin, J=7.5 Hz, 1H), 2.82-3.23 (m, 2H), 2.64-2.80 (m, 5H), 2.11-2.36 (m, 3H), 1.80-2.08 (m, 4H), 1.67 (s, 3H), 1.45-1.64 (m, 1H); MS(ES⁺) m/z 454 (MH⁺).

### Intermediate G1: 6-amino-3-chloro-2-(((1r,3r)-3-(pyrrolidin-1-yl)cyclobutyl)sulfonyl)phenol

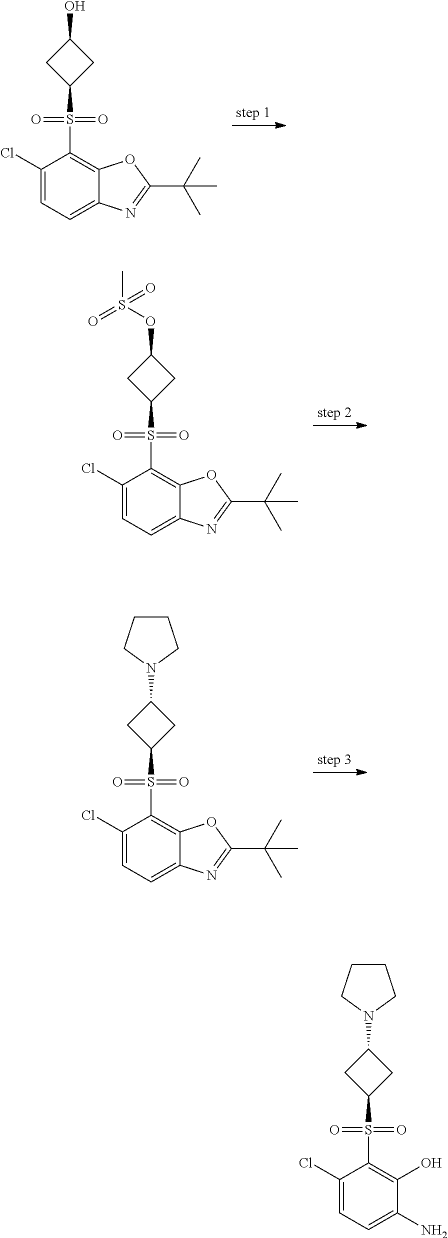 [SMILES: CC(C)(C)c1nc2ccc(Cl)c(S(=O)(=O)[C@H]3C[C@@H](O)C3)c2o1 ; CC(C)(C)c1nc2ccc(Cl)c(S(=O)(=O)[C@H]3C[C@@H](OS(C)(=O)=O)C3)c2o1 ; CC(C)(C)c1nc2ccc(Cl)c(S(=O)(=O)[C@H]3C[C@H](N4CCCC4)C3)c2o1 ; Nc1ccc(Cl)c(S(=O)(=O)[C@H]2C[C@H](N3CCCC3)C2)c1O]

Step 1: To a solution of cis-3-((2-(tert-butyl)-6-chlorobenzo[d]oxazol-7-yl)sulfonyl)cyclobutanol (Intermediate G2, 3.0 g) in DCM (30 mL) was added TEA (2.6 g) and methanesulfonyl chloride (1.2 g) at 0° C. under a nitrogen atmosphere. The resulting mixture was stirred at this temperature for 30 min. The mixture was quenched with aq. NaHCO₃ solution, extracted with DCM (2×30 mL). The combined organic phases were washed, dried and concentrated to afford cis-3-((2-(tert-butyl)-6-chlorobenzo[d]oxazol-7-yl)sulfonyl)cyclobutyl methanesulfonate (2.6 g). MS(ES⁺) m/z 422 (MH⁺).

Step 2: To a solution of cis-3-((2-(tert-butyl)-6-chlorobenzo[d]oxazol-7-yl)sulfonyl)cyclobutyl methanesulfonate (2.4 g) in DMF (30 mL) was added potassium carbonate (1.6 g) and pyrrolidine (0.5 g). The mixture was stirred at 80° C. overnight. Water (20 mL) was added. The crude product was extracted with EA (3×80 mL). The combined organic phases were washed with brine, filtered and concentrated to give the crude product, which was purified by column chromatography (eluting with PE:EA=2:1) to 2-(tert-butyl)-6-chloro-7-((trans-3-(pyrrolidin-1-yl)cyclobutyl)sulfonyl)benzo[d]oxazole (2.0 g). MS(ES⁺) m/z 397 (MH⁺).

Step 3: 2-(Tert-butyl)-6-chloro-7-((trans-3-(pyrrolidin-1-yl)cyclobutyl)sulfonyl)benzo[d]oxazole (2.0 g) was dissolved in HCl in dioxane (50 mL) and water (50 mL). The reaction mixture was stirred overnight at 120° C. The solvent was removed to give the crude product (1.5 g), which was combined with another batch of the same reaction using 2-(tert-butyl)-6-chloro-7-((trans-3-(pyrrolidin-1-yl)cyclobutyl)sulfonyl)benzo[d]oxazole (1.3 g) as starting material. The mixture was purified by preparative HPLC to afford the title compound (614 mg). MS(ES⁺) m/z 331 (MH⁺).

### Intermediate G2: cis-3-((2-(tert-butyl)-6-chlorobenzo[d]oxazol-7-yl)sulfonyl)cyclobutanol

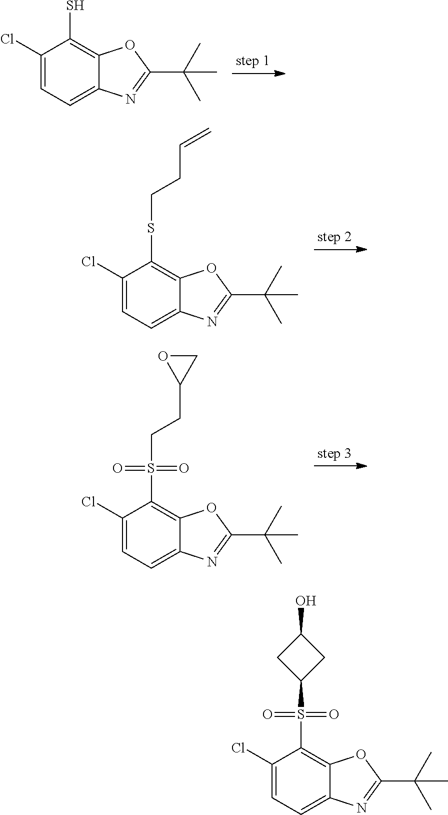

Step 1: To a solution of 2-(tert-butyl)-6-chlorobenzo[d]oxazole-7-thiol (25.0 g) in DMF (250 mL) was added 4-bromobut-1-ene (16.8 g) and then K₂CO₃ (21.4 g). The resulting mixture was stirred at 60° C. for 4 hours. After cooling, it was poured into water (1 L), and extracted with EA (2×200 mL). The combined organic layers were washed with water and brine. After drying over Na₂SO₄, the organic layer was concentrated to afford 7-(but-3-en-1-ylthio)-2-(tert-butyl)-6-chlorobenzo[d]oxazole (27.6 g). MS(ES⁺) m/z 296 (MH⁺).

Step 2: To an ice-water cooled solution of 7-(but-3-en-1-ylthio)-2-(tert-butyl)-6-chlorobenzo[d]oxazole (27.6 g) in DCM (400 mL) was added mCPBA (84.0 g) portionwise. After stirring at RT overnight, aq. NaHCO₃ and aq. Na₂S₂O₃ solutions were added. The mixture was extracted with DCM (2×500 mL). The combined organic layers were washed with water and brine, dried over Na₂SO₄, filtered and concentrated. The crude product was purified by column chromatography (eluting with PE:EA=1:0 to 7:3) to afford 2-(tert-butyl)-6-chloro-7-((2-(oxiran-2-yl)ethyl)sulfonyl)benzo[d]oxazole (26.0 g).

Step 3: To a solution of 2-(tert-butyl)-6-chloro-7-((2-(oxiran-2-yl)ethyl)sulfonyl)benzo[d]oxazole (10.0 g) in THF (200 mL) was added methylmagnesium bromide (3 M in ether, 38.8 mL) at −70° C. The mixture was warmed up slowly and stirred at RT overnight. The reaction mixture was poured into aq. NH₄Cl solution, and extracted with EA (2×200 mL). The combined organic layers were washed with brine, dried over Na₂SO₄ and filtered. After removal of solvent, the crude product was purified by column chromatography (eluting with PE:EA=4:1 to 3:2) to afford cis-3-((2-(tert-butyl)-6-chlorobenzo[d]oxazol-7-yl)sulfonyl)cyclobutanol (7.2 g).

### Compound of Formula (H): 1-(4-chloro-3-((trans-3-(dimethylamino)cyclobutyl)sulfonyl)-2-hydroxyphenyl)-3-((R)-2-methylcyclopent-2-en-1-yl)urea, trifluoroacetic acid salt

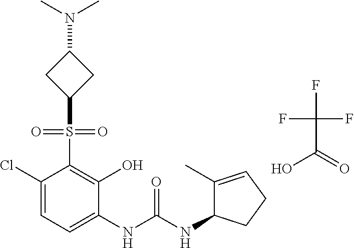

To a solution of 6-amino-3-chloro-2-((trans-3-(dimethylamino)cyclobutyl)sulfonyl)phenol (Intermediate H1, 100 mg) in pyridine (5 mL) was added fresh (R)-5-isocyanato-1-methylcyclopent-1-ene (Intermediate F2, 40 mg) in toluene (5 mL). The reaction mixture was stirred at RT overnight, and then quenched with water (5 mL). The solvent was removed. The residue was purified by MDAP (acidic condition) to afford 1-(4-chloro-3-((trans-3-(dimethylamino)cyclobutyl)sulfonyl)-2-hydroxyphenyl)-3-((R)-2-methylcyclopent-2-en-1-yl)urea as a trifluoroacetic acid salt (40 mg). ¹H-NMR (400 MHz, DMSO-d₆) δ ppm 10.46 (br. s., 1H), 8.32 (s, 1H), 8.16 (d, J=8.8 Hz, 1H), 7.06-7.17 (m, 2H), 5.52 (s, 1H), 4.50-4.58 (m, 1H), 4.40-4.50 (m, 1H), 3.98 (quin, J=7.9 Hz, 1H), 2.62-2.82 (m, 10H), 2.11-2.36 (m, 3H), 1.67 (s, 3H), 1.49-1.59 (m, 1H); MS(ES⁺) m/z 428 (MH⁺).

### Intermediate H1: 6-amino-3-chloro-2-((trans-3-(dimethylamino)cyclobutyl)sulfonyl)phenol

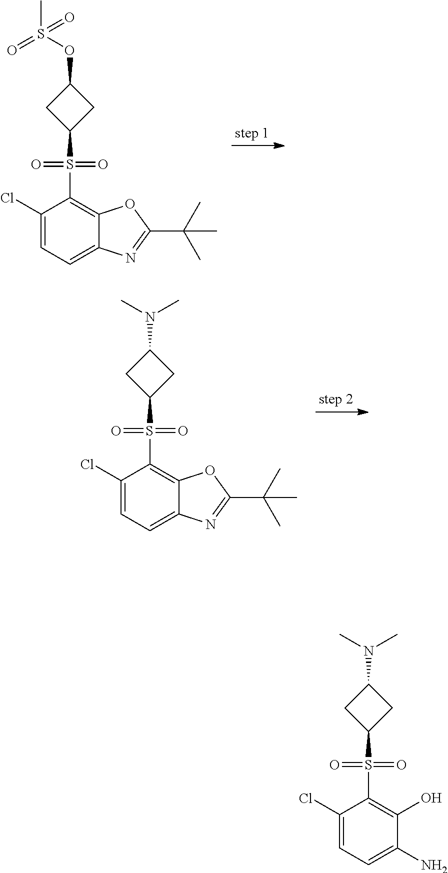

Step 1: Potassium carbonate (262 mg) was added to a solution of cis-3-((2-(tert-butyl)-6-chlorobenzo[d]oxazol-7-yl)sulfonyl)cyclobutyl methanesulfonate (Intermediate H2; 200 mg) and dimethylamine hydrochloride (77 mg) in DMF (3 mL) at RT. The reaction mixture was stirred at 100° C. for 12 hours, and then combined with another batch of the same reaction using cis-3-((2-(tert-butyl)-6-chlorobenzo[d]oxazol-7-yl)sulfonyl)cyclobutyl methanesulfonate (200 mg) as starting material. The combined mixture was diluted with EA (50 mL). The organic phase was washed with water (2×20 mL) and brine (20 mL), dried over Na₂SO₄ and concentrated to give trans-3-((2-(tert-butyl)-6-chlorobenzo[d]oxazol-7-yl)sulfonyl)-N,N-dimethylcyclobutanamine (250 mg) as a brown oil. MS(ES⁺) m/z 371 (MH⁺).

Step 2: Aq. HCl solution (35%, 5 mL) was added to a solution of trans-3-((2-(tert-butyl)-6-chlorobenzo[d]oxazol-7-yl)sulfonyl)-N,N-dimethylcyclobutanamine (550 mg) in 1,4-dioxane (10 mL) and water (10 mL) at RT. The reaction mixture was stirred at 120° C. for 3 hours, and then combined with another batch of the same reaction using trans-3-((2-(tert-butyl)-6-chlorobenzo[d]oxazol-7-yl)sulfonyl)-N,N-dimethylcyclobutanamine (400 mg) as starting material. The combined mixture was concentrated. The residue was dissolved in MeOH. Aq. NaHCO₃ solution was added until pH=8. The mixture was concentrated. The resulting residue was purified by column chromatography (eluting with DCM:MeOH=50:1) to afford the title compound (220 mg) as a black oil. MS(ES⁺) m/z 305 (MH⁺).

### Intermediate H2: cis-3-((2-(tert-butyl)-6-chlorobenzo[d]oxazol-7-yl)sulfonyl)cyclobutyl methanesulfonate

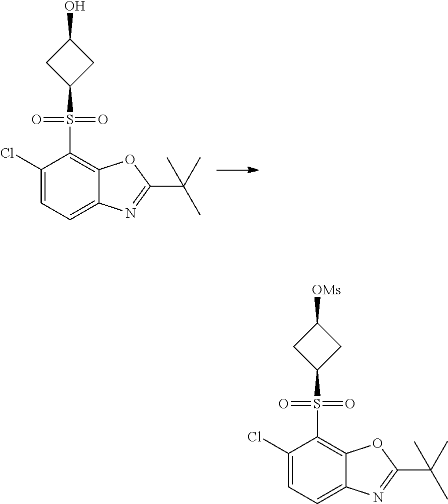 [SMILES: CC(C)(C)c1nc2ccc(Cl)c(S(=O)(=O)[C@H]3C[C@@H](O)C3)c2o1 ; CC(C)(C)c1nc2ccc(Cl)c(S(=O)(=O)[C@H]3C[C@@H](OS(C)(=O)=O)C3)c2o1]

To a solution of cis-3-((2-(tert-butyl)-6-chlorobenzo[d]oxazol-7-yl)sulfonyl)cyclobutanol (Intermediate G2, 1.0 g) and N,N-diethylpropan-2-amine (0.9 mL) in DCM (20 mL) was added methanesulfonyl chloride (0.3 mL) at 0° C. The mixture was stirred at 0° C. for 4 hours. The reaction mixture was then poured into water and extracted with DCM (2×50 mL). The combined organic phases were washed, dried and concentrated to give cis-3-((2-(tert-butyl)-6-chlorobenzo[d]oxazol-7-yl)sulfonyl)cyclobutyl methanesulfonate (1.3 g) as a colorless solid. MS(ES⁺) m/z 422 (MH⁺).

### Compound of Formula (I): (R)-1-(4-chloro-3-((1,1-difluoroethyl)sulfonyl)-2-hydroxyphenyl)-3-(2-methylcyclopent-2-en-1-yl)urea

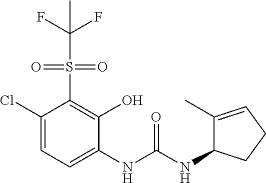 [SMILES: CC1=CCC[C@H]1NC(=O)Nc1ccc(Cl)c(S(=O)(=O)C(C)(F)F)c1O]

To a solution of 6-amino-3-chloro-2-((1,1-difluoroethyl)sulfonyl)phenol (Intermediate I1, 84 mg) in pyridine (5 mL) was added (R)-5-isocyanato-1-methylcyclopent-1-ene (Intermediate F2, 0.15 M in toluene, 4 mL). The reaction mixture was stirred at RT overnight. The mixture was concentrated and the residue was dissolved in DMF (8 mL) and purified by MDAP to afford (R)-1-(4-chloro-3-((1,1-difluoroethyl)sulfonyl)-2-hydroxyphenyl)-3-(2-methylcyclopent-2-en-1-yl)urea (15 mg) as a white solid. ¹H-NMR (400 MHz, DMSO-d₆) δ ppm 10.53 (br. s., 1H), 8.28 (s, 1H), 8.26 (d, J=9.0 Hz, 1H), 7.17 (d, J=8.8 Hz, 1H), 7.07 (d, J=8.3 Hz, 1H), 5.52 (br. s., 1H), 4.44-4.62 (m, 1H), 2.23-2.36 (m, 2H), 2.16-2.23 (m, 1H), 2.09 (t, J=19.2 Hz, 3H), 1.67 (s, 3H), 1.47-1.59 (m, 1H); MS(ES⁺) m/z 395 (MH⁺).

### Intermediate I1: 6-amino-3-chloro-2-((1,1-difluoroethyl)sulfonyl)phenol

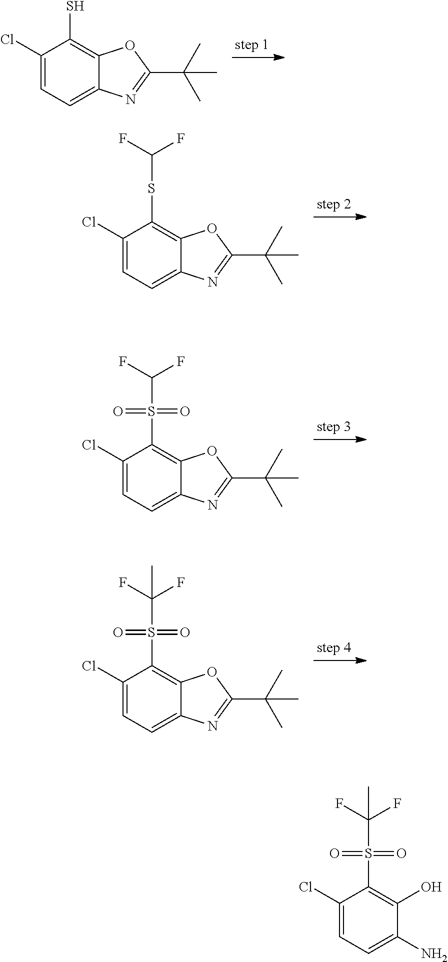

Step 1: To a solution of 2-(tert-butyl)-6-chlorobenzo[d]oxazole-7-thiol (6.0 g) and potassium hydroxide (13.9 g) in acetonitrile (80 mL) and water (80 mL) stirred at −78° C. was added diethyl (bromodifluoromethyl)phosphonate (11.9 g) in one portion. The mixture was allowed to warm to RT and stirred for 30 min. EA (200 mL) was added. The organic phase was separated. The aqueous phase was extracted with EA (2×100 mL). The organic layers were combined, dried over sodium sulfate, filtered and concentrated to afford 2-(tert-butyl)-6-chloro-7-((difluoromethyl)thio)benzo[d]oxazole (7.2 g) as a colorless oil. MS(ES⁺) m/z 292 (MH⁺).

Step 2: To a solution of 2-(tert-butyl)-6-chloro-7-((difluoromethyl)thio)benzo[d]oxazole (7.2 g) in DCM (200 mL) at 0° C. was added mCPBA (19.5 g) and stirred for 2 hours. As the starting material was not consumed totally, additional mCPBA (19.5 g) was added. The mixture was stirred at RT overnight. Then aq. Na₂SO₃ solution was added. The organic phase was separated, washed with sat. sodium carbonate solution and brine. The resulting solution was dried over sodium sulfate, filtered and concentrated. The residue was purified by column chromatography (eluting with PE:EA=1:0-2:3) to give 2-(tert-butyl)-6-chloro-7-((difluoromethyl)sulfonyl)benzo[d]oxazole (3.2 g) as a white solid. MS(ES⁺) m/z 324 (MH⁺).

Step 3: To a solution of 2-(tert-butyl)-6-chloro-7-((difluoromethyl)sulfonyl)benzo[d]oxazole (2.2 g) and iodomethane (4.2 mL) in THF (30 mL) and HMPA (27 mL) was added LDA (2 M in THF, 13.5 mL). The mixture was stirred at −50° C. for 30 min. The mixture was then neutralized with sat. NH₄Cl solution and 10% HCl solution. EA (50 mL) was added. The organic layers were washed with brine, dried over sodium sulfate and concentrated in vacuo. The crude product was combined with another batch of the same reaction using 2-(tert-butyl)-6-chloro-7-((difluoromethyl)sulfonyl)benzo[d]oxazole (1.0 g) as starting material. The crude product was purified by column chromatography (eluting with PE:EA=1:0-3:2) to afford 2-(tert-butyl)-6-chloro-7-((1,1-difluoroethyl)sulfonyl)benzo[d]oxazole (1.0 g) as a white solid. MS(ES⁺) m/z 338 (MH⁺).

Step 4: To a solution of 2-(tert-butyl)-6-chloro-7-((1,1-difluoroethyl)sulfonyl)benzo[d]oxazole (1.0 g) in 1,4-dioxane (20 mL) was added conc. HCl solution (20 mL). The mixture was refluxed at 110° C. for 4 hours, and then concentrated. The resulting residue was dissolved in EA (20 mL). The pH of the solution was adjusted to 8 with TEA. The mixture was concentrated. The residue was purified by column chromatography (eluting with PE:EA=1:0 to 1:4) to afford the title compound (1.0 g) as a light brown solid. MS(ES⁺) m/z 272 (MH⁺).

### Compound of Formula (J): 1-(4-chloro-2-hydroxy-3-(((S)-3-methyltetrahydrofuran-3-yl)sulfonyl)phenyl)-3-((R)-2-methylcyclopent-2-en-1-yl)urea

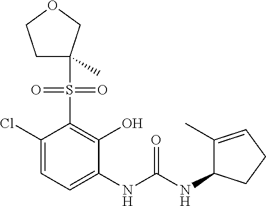 [SMILES: CC1=CCC[C@H]1NC(=O)Nc1ccc(Cl)c(S(=O)(=O)[C@@]2(C)CCOC2)c1O]

### and Compound of Formula (C): 1-(4-chloro-2-hydroxy-3-(((R)-3-methyltetrahydrofuran-3-yl)sulfonyl)phenyl)-3-((R)-2-methylcyclopent-2-en-1-yl)urea

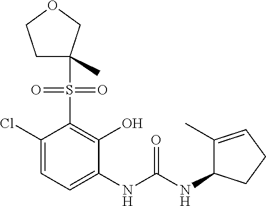 [SMILES: CC1=CCC[C@H]1NC(=O)Nc1ccc(Cl)c(S(=O)(=O)[C@]2(C)CCOC2)c1O]

To a solution of 6-amino-3-chloro-2-((3-methyltetrahydrofuran-3-yl)sulfonyl)phenol (Intermediate JC1, 3000 mg) in pyridine (20 mL) was added (R)-5-isocyanato-1-methylcyclopent-1-ene (Intermediate F2, 2279 mg). The resulting mixture was stirred at 40° C. for 12 hours. Cold water (30 mL) was added and aqueous layer was extracted with DCM (2×100 mL). The combined organic layers were dried over Na₂SO₄, filtered and concentrated to give 1-(4-chloro-2-hydroxy-3-((3-methyltetrahydrofuran-3-yl)sulfonyl)phenyl)-3-((R)-2-methylcyclopent-2-en-1-yl)urea (4.1 g) as a dark solid. A part of this compound (3.0 g) was purified by SFC and MDAP under acidic condition to afford the pure two enantiomers: 1-(4-chloro-2-hydroxy-3-(((S)-3-methyltetrahydrofuran-3-yl)sulfonyl)phenyl)-3-((R)-2-methylcyclopent-2-en-1-yl)urea (Compound of Formula (J), 666 mg) and 1-(4-chloro-2-hydroxy-3-(((R)-3-methyltetrahydrofuran-3-yl)sulfonyl)phenyl)-3-((R)-2-methylcyclopent-2-en-1-yl)urea (Compound of Formula (C), 626 mg).

Compound of Formula (J): HPLC (Chiralpak IC column (4.6*250 mm, 5 uM), 1:1 ACN/IPA (containing 0.5% DEA), CO₂ flow rate: 2.55 mL/min; co-solvent flow rate: 0.45 mL/min; back pressure: 120 bar); tᵣ=16.9 min; >93% ee; ¹H-NMR (400 MHz, DMSO-d₆) δ ppm 10.53 (s, 1H), 8.36 (d, J=8.8 Hz, 1H), 8.17 (s, 1H), 7.14 (d, J=8.8 Hz, 1H), 7.05 (d, J=8.3 Hz, 1H), 5.51 (s, 1H), 4.49-4.63 (m, 1H), 4.33 (d, J=10.3 Hz, 1H), 3.76-3.89 (m, 2H), 3.61 (d, J=10.0 Hz, 1H), 2.68 (dt, J=13.3, 7.9 Hz, 1H), 2.10-2.35 (m, 3H), 1.91-2.02 (m, 1H), 1.67 (s, 3H), 1.44-1.58 (m, 4H); MS(ES⁺) m/z 415 (MH⁺);

Compound of Formula (C): HPLC (Chiralpak IC column (4.6*250 mm, 5 uM), 1:1 ACN/IPA (containing 0.5% DEA), CO₂ flow rate: 2.55 mL/min; co-solvent flow rate: 0.45 mL/min; back pressure: 120 bar); tᵣ=14.3 min; >99% ee; ¹H-NMR (400 MHz, DMSO-d₆) δ ppm 10.52 (s, 1H), 8.36 (d, J=9.0 Hz, 1H), 8.17 (s, 1H), 7.14 (d, J=8.8 Hz, 1H), 7.05 (d, J=8.3 Hz, 1H), 5.51 (s, 1H), 4.49-4.61 (m, 1H), 4.33 (d, J=10.0 Hz, 1H), 3.75-3.91 (m, 2H), 3.61 (d, J=10.0 Hz, 1H), 2.68 (dt, J=13.3, 7.9 Hz, 1H), 2.09-2.37 (m, 3H), 1.90-2.02 (m, 1H), 1.67 (s, 3H), 1.42-1.59 (m, 4H); MS(ES⁺) m/z 415 (MH⁺);

### Intermediate JC1: 6-amino-3-chloro-2-((3-methyltetrahydrofuran-3-yl)sulfonyl)phenol

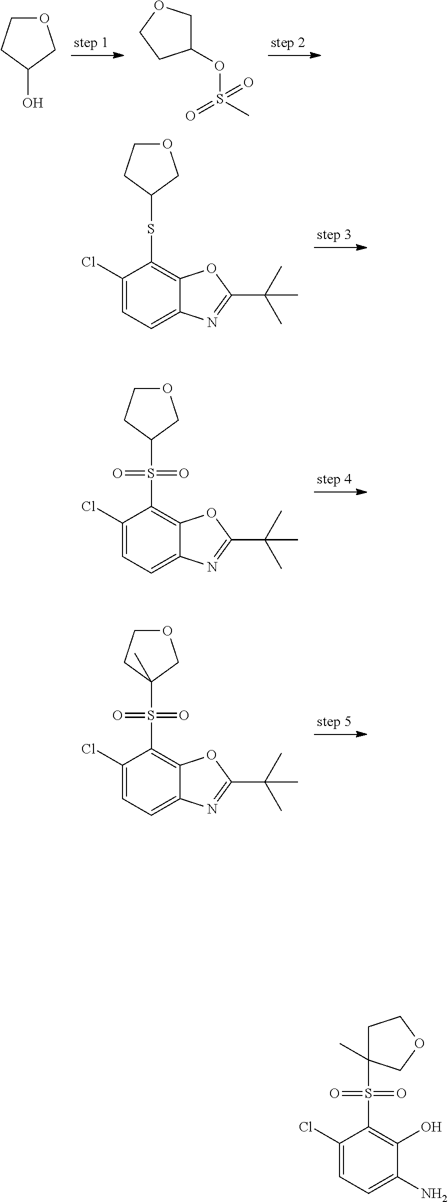

Step 1: To an ice-water cooled solution of tetrahydrofuran-3-ol (5.0 g) in DCM (100 mL) was added TEA (11.9 mL) and MsCl (4.9 mL). The resulting reaction mixture was stirred at 0° C. for 3 hours, and then quenched with aq. NaHCO₃ solution. The mixture was extracted with EA (2×100 mL). The combined organic phases were washed, dried, filtered and concentrated to afford tetrahydrofuran-3-yl methanesulfonate (6.2 g).

Step 2: To a solution of sodium 2-(tert-butyl)-6-chlorobenzo[d]oxazole-7-thiolate (11.8 g) in DMF (100 mL) was added tetrahydrofuran-3-yl methanesulfonate (6.2 g) and K₂CO₃ (7.7 g). The resulting reaction mixture was stirred at 80° C. overnight. After cooling, it was poured into water (500 mL) and extracted with EA (2×150 mL). The combined organic phases were washed, dried and concentrated to afford 2-(tert-butyl)-6-chloro-7-((tetrahydrofuran-3-yl)thio)benzo[d]oxazole (11.4 g).

Step 3: To an ice-water cooled solution of 2-(tert-butyl)-6-chloro-7-((tetrahydrofuran-3-yl)thio)benzo[d]oxazole (7.4 g) in DCM (200 mL) was added mCPBA (11.7 g). The resulting mixture was stirred at RT for over 2 days, and then quenched with aq. NaHCO₃ solution and aq. Na₂S₂O₃ solution. The mixture was extracted with EA (2×200 mL). The combined organic phases were washed, dried and concentrated. The residue was purified by column chromatography (eluting with 0-40% EA in PE) to afford 2-(tert-butyl)-6-chloro-7-((tetrahydrofuran-3-yl)sulfonyl)benzo[d]oxazole (4.3 g). MS(ES⁺) m/z 344 (MH⁺).

Step 4: To a dry ice-ethanol cooled solution of 2-(tert-butyl)-6-chloro-7-((tetrahydrofuran-3-yl)sulfonyl)benzo[d]oxazole (4.3 g) and MeI (2.0 mL) in THF (100 mL) was added LiHMDS (1 M in THF, 31.3 mL). The resulting mixture was warmed up slowly and stirred at RT for 30 min. Aq. NH₄Cl solution was added. The mixture was extracted with EA (2×150 mL). The combined organic phases were washed, dried and concentrated to afford 2-(tert-butyl)-6-chloro-7-((3-methyltetrahydrofuran-3-yl)sulfonyl)benzo[d]oxazole (4.4 g). MS(ES⁺) m/z 358 (MH⁺).

Step 5: To a solution of 2-(tert-butyl)-6-chloro-7-((3-methyltetrahydrofuran-3-yl)sulfonyl)benzo[d]oxazole (4.4 g) in 1,4-dioxane (150 mL) was added aq. HCl solution (37%, 30.3 mL). The mixture was refluxed at 120° C. overnight, and then concentrated. The residue was dissolved in water (100 mL). The pH of the solution was adjusted to 8 with aq. NaHCO₃ solution, and extracted with EA. The organic phase was washed and concentrated. The resulting residue was purified by column chromatography (eluting with a gradient of 0-80% EA in PE) to afford the title compound (2.4 g). MS(ES⁺) m/z 292 (MH⁺).

### Compound of Formula (K): 1-(4-chloro-2-hydroxy-3-(((S)-3-methyltetrahydrofuran-3-yl)sulfonyl)phenyl)-3-((R)-2-chlorocyclopent-2-en-1-yl)urea and Compound of Formula (L): 1-(4-chloro-2-hydroxy-3-(((R)-3-methyltetrahydrofuran-3-yl)sulfonyl)phenyl)-3-((R)-2-chlorocyclobent-2-en-1-yl)urea

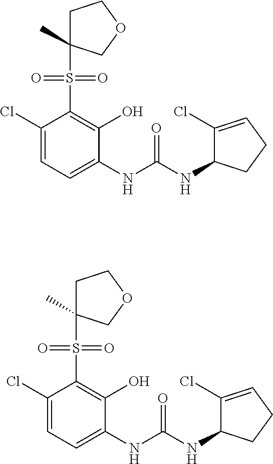 [SMILES: C[C@@]1(S(=O)(=O)c2c(Cl)ccc(NC(=O)N[C@@H]3CCC=C3Cl)c2O)CCOC1 ; C[C@]1(S(=O)(=O)c2c(Cl)ccc(NC(=O)N[C@@H]3CCC=C3Cl)c2O)CCOC1]

To a solution of 6-amino-3-chloro-2-((3-methyltetrahydrofuran-3-yl)sulfonyl)phenol (Intermediate JC1, 600 mg) in pyridine (20 mL) was added the fresh (R)-1-chloro-5-isocyanatocyclopent-1-ene (Intermediate E2, 443 mg) in toluene (20 mL). The mixture was stirred at RT overnight. The resulting solution was quenched with water (5 mL) and concentrated. The residue was purified with MDAP (acidic condition) to afford 1-(4-chloro-2-hydroxy-3-((3-methyltetrahydrofuran-3-yl)sulfonyl)phenyl)-3-((R)-2-chlorocyclopent-2-en-1-yl)urea (205 mg). A part of this compound (160 mg) was purified by chiral separation (AD-H (4.6*250 mm,5 um), co-solvent MeOH, 1% DEA) and then MDAP (acidic condition) to give 1-(4-chloro-2-hydroxy-3-(((S)-3-methyltetrahydrofuran-3-yl)sulfonyl)phenyl)-3-((R)-2-chlorocyclopent-2-en-1-yl)urea and 1-(4-chloro-2-hydroxy-3-(((R)-3-methyltetrahydrofuran-3-yl)sulfonyl)phenyl)-3-((R)-2-chlorocyclopent-2-en-1-yl)urea (Compound of Formula (K) and Compound of Formula (L), 37 mg and 31 mg, respectively) as violet solids.

Isomer 1: HPLC (AD-H column (4.6*250 mm, 5 uM), IPA (containing 0.1% DEA), CO₂ flow rate: 2.25 mL/min; co-solvent flow rate: 0.75 mL/min; back pressure: 149 bar); tᵣ=4.7 min; >99% ee; ¹H-NMR (400 MHz, DMSO-d₆) δ ppm 10.49 (s, 1H), 8.37 (d, J=8.9 Hz, 1H), 8.25 (s, 1H), 7.38 (d, J=8.8 Hz, 1H), 7.16 (d, J=8.9 Hz, 1H), 5.99 (d, J=1.7 Hz, 1H), 4.73 (s, 1H), 4.34 (d, J =10.1 Hz, 1H), 3.72-3.97 (m, 2H), 3.61 (d, J=10.1 Hz, 1H), 2.67 (m, 1H), 2.18-2.47 (m, 3H), 1.84-2.04 (m, 1H), 1.57-1.78 (m, 1H), 1.49 (s, 3H); MS(ES⁺) m/z 435 (MH⁺).

Isomer 2: HPLC (AD-H column (4.6*250 mm, 5 uM), IPA (containing 0.1% DEA), CO₂ flow rate: 2.25 mL/min; co-solvent flow rate: 0.75 mL/min; back pressure: 149 bar); tᵣ=5.5 min; >93% ee; ¹H-NMR (400 MHz, DMSO-d₆) δ ppm 10.49 (s, 1H), 8.37 (d, J=8.9 Hz, 1H), 8.24 (s, 1H), 7.37 (d, J=8.7 Hz, 1H), 7.16 (d, J=8.9 Hz, 1H), 5.99 (s, 1H), 4.73 (s, 1H), 4.34 (d, J=10.1 Hz, 1H), 3.71-3.96 (m, 2H), 3.61 (d, J=10.1 Hz, 1H), 2.62-2.78 (m, 1H), 2.19-2.44 (m, 3H), 1.84-2.06 (m, 1H), 1.56-1.78 (m, 1H), 1.49 (s, 3H); MS(ES⁺) m/z 435 (MH⁺).

1-25. (canceled)

26. A method for prevention and/or treatment of CIPN in a human in need thereof comprising administering a CXCR2 antagonist.

27. The method according to claim 26, wherein the method is for prevention of CIPN.

28. The method according to claim 26, wherein the CIPN is caused by a platinum-containing chemotherapeutic agent

29. The method according to claim 26, wherein the CIPN is caused by oxaliplatin.

30. The method according to claim 26, wherein the CXCR2 antagonist has a pIC50 value against the CXCR2 receptor of greater than or equal to 7.0 as measured in a CXCR2 receptor binding assay.

31. The method according to claim 26, wherein the CXCR2 antagonist has a pIC50 value against the CXCR2 receptor of greater than or equal to 7.0 as measured in assay a).

32. The method according to claim 26, wherein the CXCR2 antagonist has a pA2 value against the CXCR2 receptor of greater than or equal to 8.0 as measured in assay b).

33. The method according to claim 26, wherein the CXCR2 antagonist has a pIC50 value against the CXCR2 receptor of greater than or equal to 8.0 as measured in assays c).

34. The method according to claim 26, wherein the CXCR2 antagonist has a pIC50 value against the CXCR2 receptor of greater than or equal to 5.0 as measured in assay d)

35. The method according to claim 26, wherein the CXCR2 antagonist has a selectivity for CXCR2 over CXCR1 of greater than or equal to 29 fold.

36. The method according to claim 26, wherein the CXCR2 antagonist has solubility of 10 μg/ml.

37. The method according to claim 26, wherein the CXCR2 antagonist has a membrane permeability of Log D_{7.4} of −2 to +4.

38. The method according to claim 26, wherein the CXCR2 antagonist is selected from the group consisting of:
1-(4-chloro-2-hydroxy-3-(piperazin-1-ylsulfonyl)phenyl)-3-(2-chloro-3-fluorophenyl)urea of Formula (A) 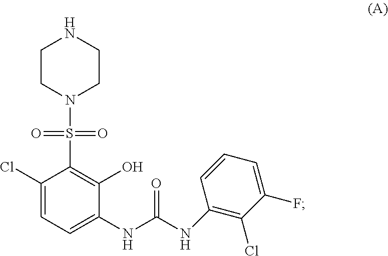 [SMILES: O=C(Nc1ccc(Cl)c(S(=O)(=O)N2CCNCC2)c1O)Nc1cccc(F)c1Cl]
1-(4-chloro-2-hydroxy-3-(piperidin-3-ylsulfonyl)phenyl)-3-(3-fluoro-2-methylphenyl)urea of Formula (B) 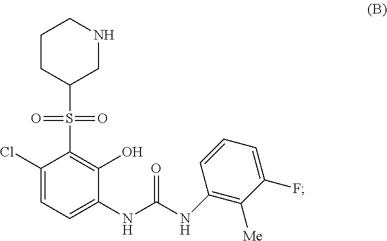 [SMILES: Cc1c(F)cccc1NC(=O)Nc1ccc(Cl)c(S(=O)(=O)C2CCCNC2)c1O]
1-(4-chloro-2-hydroxy-3-(((R)-3-methyltetrahydrofuran-3-yl)sulfonyl)phenyl)-3-((R)-2-methylcyclopent-2-en-1-yl)urea of Formula (C) 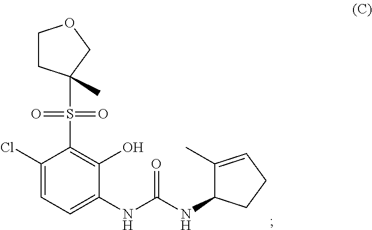 [SMILES: CC1=CCC[C@H]1NC(=O)Nc1ccc(Cl)c(S(=O)(=O)[C@]2(C)CCOC2)c1O]
1-(4-chloro-3-((1,4-dimethylpiperidin-4-yl)sulfonyl)-2-hydroxyphenyl)-3-(2-chloro-3-fluorophenyl)urea of Formula (D) 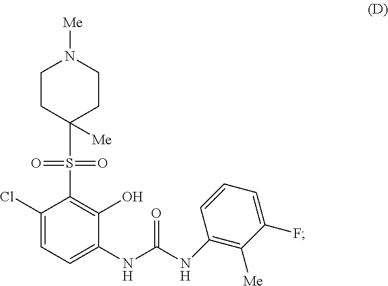 [SMILES: Cc1c(F)cccc1NC(=O)Nc1ccc(Cl)c(S(=O)(=O)C2(C)CCN(C)CC2)c1O]
(R)-1-(4-chloro-2-hydroxy-3-((4-methyltetrahydro-2H-pyran-4-yl)sulfonyl)phenyl)-3-(2-chlorocyclopent-2-en-1-yl)urea of Formula (E) 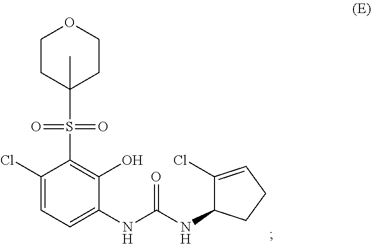
(R)-1-(3-(tert-butylsulfonyl)-4-cyano-2-hydroxyphenyl)-3-(2-methylcyclopent-2-en-1-yl)urea of Formula (F) 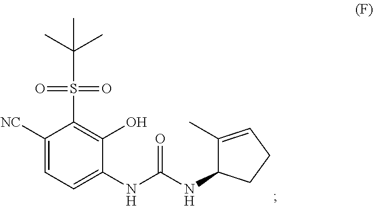 [SMILES: CC1=CCC[C@H]1NC(=O)Nc1ccc(C#N)c(S(=O)(=O)C(C)(C)C)c1O]
1-(4-chloro-2-hydroxy-3-((trans-3-(pyrrolidin-1-yl)cyclobutyl)sulfonyl)phenyl)-3-((R)-2-methylcyclopent-2-en-1-yl)urea of Formula (G) 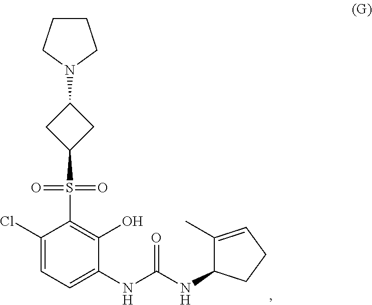 [SMILES: CC1=CCC[C@H]1NC(=O)Nc1ccc(Cl)c(S(=O)(=O)[C@H]2C[C@H](N3CCCC3)C2)c1O]
1-(4-chloro-3-((trans-3-(dimethylammo)cyclobutyl)sulfonyl)-2-hydroxyphenyl)-3-((R)-2-methylcyclopent-2-en-1-yl)urea of Formula (H) 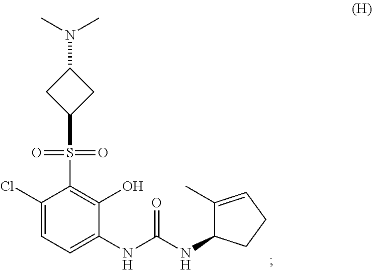 [SMILES: CC1=CCC[C@H]1NC(=O)Nc1ccc(Cl)c(S(=O)(=O)[C@H]2C[C@H](N(C)C)C2)c1O]
(R)-1-(4-chloro-3-((1,1-difluoroethyl)sulfonyl)-2-hydroxyphenyl)-3-(2-methylcyclopent-2-en-1-yl)urea of Formula (I) 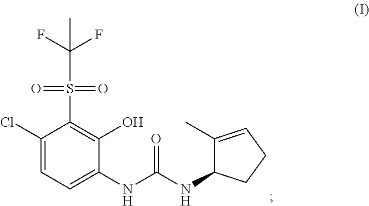 [SMILES: CC1=CCC[C@H]1NC(=O)Nc1ccc(Cl)c(S(=O)(=O)C(C)(F)F)c1O]
1-(4-chloro-2-hydroxy-3-(((S)-3-methyltetrahydrofuran-3-yl)sulfonyl)phenyl)-3-((R)-2-methylcyclopent-2-en-1-yl)urea of Formula (J) 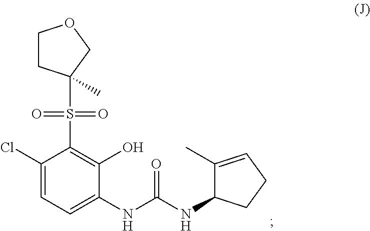 [SMILES: CC1=CCC[C@H]1NC(=O)Nc1ccc(Cl)c(S(=O)(=O)[C@@]2(C)CCOC2)c1O]
1-(4-chloro-2-hydroxy-3-(((S)-3-methyltetrahydrofuran-3-yl)sulfonyl)phenyl)-3-((R)-2-chlorocyclopent-2-en-1-yl)urea of Formula (K) 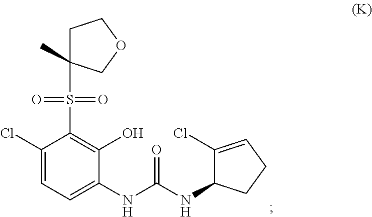 [SMILES: C[C@@]1(S(=O)(=O)c2c(Cl)ccc(NC(=O)N[C@@H]3CCC=C3Cl)c2O)CCOC1]
and
1-(4-chloro-2-hydroxy-3-(((R)-3-methyltetrahydrofuran-3-yl)sulfonyl)phenyl)-3-((R)-2-chlorocyclopent-2-en-1-yl)urea of Formula (L) 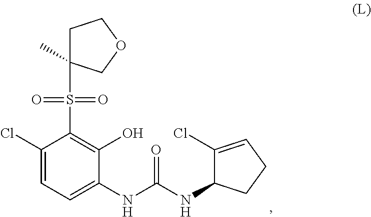 [SMILES: C[C@]1(S(=O)(=O)c2c(Cl)ccc(NC(=O)N[C@@H]3CCC=C3Cl)c2O)CCOC1]
or a pharmaceutically acceptable salt thereof.

39. The method according to claim 26, wherein the CXCR2 antagonist is 1-(4-chloro-2-hydroxy-3-(piperazin-1-ylsulfonyl)phenyl)-3-(2-chloro-3-fluorophenyl)urea of Formula (A) or a pharmaceutically acceptable salt thereof

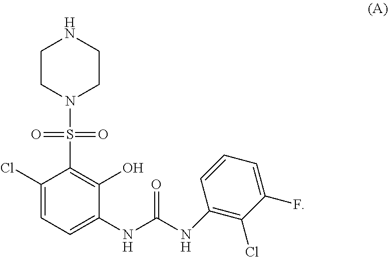 [SMILES: O=C(Nc1ccc(Cl)c(S(=O)(=O)N2CCNCC2)c1O)Nc1cccc(F)c1Cl]

40. The method according to claim 26, wherein the CXCR2 antagonist is 1-(4-chloro-2-hydroxy-3-(piperidin-3-ylsulfonyl)phenyl)-3-(3-fluoro-2-methylphenyl)urea of Formula (B) or a pharmaceutically acceptable salt thereof

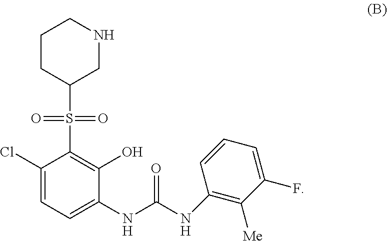 [SMILES: Cc1c(F)cccc1NC(=O)Nc1ccc(Cl)c(S(=O)(=O)C2CCCNC2)c1O]

41. The method according to claim 26, wherein the CXCR2 antagonist is 1-(4-chloro-2-hydroxy-3-(((S)-3-methyltetrahydrofuran-3-yl)sulfonyl)phenyl)-3-((R)-2-methylcyclopent-2-en-1-yl)urea of Formula (J) or a pharmaceutically acceptable salt thereof

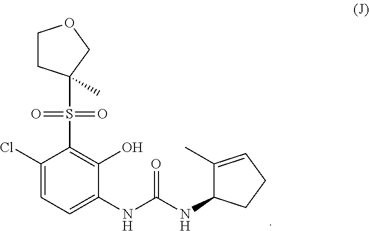 [SMILES: CC1=CCC[C@H]1NC(=O)Nc1ccc(Cl)c(S(=O)(=O)[C@@]2(C)CCOC2)c1O]

42. A combination comprising a) a CXCR2 antagonist and b) one or more primary chemotherapeutic agent of platinum compounds.

43. The combination according to claim 42, wherein the chemotherapeutic agent is selected from the group consisting of oxaliplatin, taxanes, vinca alkaloids, thalidomide and bortezomib.

44. The combination according to claim 42, wherein the primary chemotherapeutic agent is oxaliplatin.

45. The combination according to claim 42, wherein the CXCR2 antagonist is administered before or at the same time as the one or more primary chemotherapeutic agent.
